SEQUENCE LISTING (7 sequences)

SEQ ID NO: 1 - skipped

SEQ ID NO: 2 - protein, 4 aa, Homo sapiens
RGDS

SEQ ID NO: 3 - protein, 9 aa, Homo sapiens
GGGGRGDSP

SEQ ID NO: 4 - protein, 6 aa, Homo sapiens
GRGDSP

SEQ ID NO: 5 - protein, 5 aa, Homo sapiens
GRGDS

SEQ ID NO: 6 - protein, 5 aa, synthetic construct
CRGDS

SEQ ID NO: 7 - protein, 5 aa, Homo sapiens
YIGSR

(19) United States
(12) Patent Application Publication
(10) Pub. No.: US 2025/0242078 A1
(43) Pub. Date: Jul. 31, 2025

(54) NOVEL POLYMER

(71) Applicants: St. Vincent's Hospital (Melbourne) Limited, Fitzroy, Victoria (AU); University of Wollongong, Wollongong, New South Wales (AU); Swinburne University of Technology, Hawthorn, Victoria (AU); The University of Melbourne, Melbourne, Victoria (AU)

(72) Inventors: Peter F.M. Choong, Melbourne, Victoria (AU); Sanjeev Gambhir, Wollongong, New South Wales (AU); Gordon George Wallace, Wollongong, New South Wales (AU); Serena Duchi, Melbourne, Victoria (AU); Carmine Onofrillo, Melbourne, Victoria (AU); Cathal D. O'Connell, Melbourne, Victoria (AU); Claudia Di Bella, Fitzroy, Victoria (AU); Simon Edward Moulton, Hawthorn, Victoria (AU)

(21) Appl. No.: 18/714,285

(22) PCT Filed: Dec. 1, 2022
(86) PCT No.: PCT/AU2022/051439

§ 371 Date: May 29, 2024

(30) Foreign Application Priority Data
Dec. 1, 2021 (AU) 2021903886

(51) Int. Cl. A61L 27/20; A61L 27/36; A61L 27/38; A61L 27/52; B33Y 80/00

(52) U.S. Cl. CPC A61L 27/20; A61L 27/3654; A61L 27/3834; A61L 27/52; A61L 2400/06; A61L 2430/06; B33Y 80/00

(57) ABSTRACT

The present invention relates to a functionalised polymer, methods of preparing the functionalised polymer, and compositions comprising the functionalised polymer. The present invention also relates to methods of using the functionalised polymer including for forming a polymer composition comprising cells from a tissue sample and for cell therapy.

### CROSS REFERENCE TO RELATED APPLICATIONS

This application is a U.S. National Stage Application filed under 35 U.S.C. ß 371, based on International Patent Application No. PCT/AU2022/051439, filed on Dec. 1, 2022, which claims priority to AU patent application No. 2021903886, filed Dec. 1, 2021. The entire contents of each of the above applications are incorporated herein by reference.

### SEQUENCE LISTING

The present application contains a Sequence Listing XML file, which has been submitted electronically in .xml format as part of the specification, is incorporated herein by reference in its entirety. Said XML file, created on Oct. 9, 2024, is named 1247343 V2-3-0.xml, and is 6,316 bytes in size.

### FIELD OF THE INVENTION

The present invention relates to a functionalised polymer, methods of preparing the functionalised polymer, and compositions comprising the functionalised polymer. The present invention also relates to methods of using the functionalised polymer including for forming a polymer composition comprising cells from a tissue sample and for cell therapy.

### SEQUENCE LISTING

The instant application contains a Sequence Listing which has been submitted electronically in XML file format and is hereby incorporated by reference in its entirety. Said XML copy, created on Oct. 9, 2024, is named 1247343 v2-3-0.xml and is 6,316 bytes in size.

### BACKGROUND OF THE INVENTION

Technologies to isolate, culture, expand, detach and deliver cells are of broad use in biomedicine, regenerative medicine, tissue engineering and stem cell therapy. Existing technologies require the sequential processing of cells on different materials, and typically with the addition of digesting enzymes and often animal derived enzymes which may irreversibly damage cells.

The isolation, culture, expansion, detachment and preparation of a cell population for implantation currently requires a series of steps which may each degrade the therapeutic capacity of the cells. Existing techniques to isolate a stem cell population (such as human adipose derived stem cells) from a tissue explant (such as the infrapatellar fat pad) rely upon the preferential attachment of a cell population onto cell culture plastics. Following removal of the undesired components of the explant, the desired population is then typically cultured on the same plastic substrate to expand the population to a useful number. When a large enough number of cells is reached, or when the cells have grown to confluency (a state where further expansion is constrained by the available surface area) the cells are detached from the cell culture plastic. The standard technique for detaching cells is to use digesting enzymes such as trypsin, collagenase or Dispase. The detached cell population is then typically mixed with another material (for example a hydrogel) for therapeutic delivery through injection or implant delivery, or to form a bio-ink for a subsequent biofabrication or 3D bioprinting step. In injection or extrusion-based procedures, the hydrogel material is often a shear-thinning material which protects the cells against shear stress induced damage.

This standard process contains a number of elements which can reduce the therapeutic capacity of the cell population, including:

Cell culture plastics have a mechanical stiffness several orders of magnitude above that of native tissues. Growth of stem cells upon such high-stiffness materials is known to reduce the stem-like phenotype of stem cells and/or induce senescence.

The enzymatic detachment processes typically require animal derived enzymes, which may be undesirable depending on the final use of the cells. These methods typically work through cleavage of cell surface proteins leading to dysregulation of cell function. Such methods can induce apoptosis in cells when exposed for longer time periods. Such methods unavoidably disrupt cell-cell interactions, which in many cases are desired (such as tissue spheroid or organoid cultures).

Thirdly, the detachment of cells from the tissue culture plastic and transfer to a biopolymer environment inevitably causes additional loss of cells through transfer errors.

Accordingly, there is a need for new and/or improved methods and compositions for preparing cells for implantation, and more generally, for improvements in procedures that utilise re-implantation of autologous cells.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present inventors have developed functionalised polymers which are capable of functioning in a sequential process as a cell culture substrate, a bio-ink and a bio-scaffold for tissue engineering applications. The functionalised polymers may advantageously be used for all steps of the process (isolation, purification, expansion, detachment and/or delivery).

In one aspect, the present invention provides a functionalised polymer comprising a polymer, preferably an alginate polymer, partially functionalised with:
a plurality of photocrosslinkable moieties linked to the polymer, each photocrosslinkable moiety comprising a reactive functionality capable of photocrosslinking; and
a plurality of cell adhesion moieties each linked to the polymer via a linker, each cell adhesion moiety comprising a cell adhesion motif.

In preferred embodiments, the functionalised polymer further comprises a plurality of functional groups on the polymer capable of ionic crosslinking with an ionic crosslinking agent. The plurality of functional groups may comprise one or more of a hydroxyl group, a carboxyl group and an ester group, preferably a carboxyl group.

In the case of an alginate polymer, carboxyl groups are present which are crosslinked in the presence of divalent cations, particularly calcium cations (Ca²⁺).

In some embodiments, the functionalised polymer is an ionic crosslinked polymer. In some embodiments, the ionic crosslinked polymer while capable of being photocrosslinked is not photocrosslinked.

In some embodiments, the functionalised polymer may comprise the following structure:

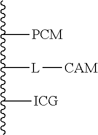

wherein the wavy line represents the polymer backbone, PCM represents a photocrosslinkable moiety, L represents a linker, CAM represents a cell adhesion moiety, and ICG represents a functional group capable of ionic crosslinking, and wherein the number of photocrosslinkable moieties, cell adhesion moieties and functional groups capable of ionic crosslinking are each independently variable.

Preferably, the functionalised polymer may have the following properties: 1. cellular adhesion, 2. inducible phase change, preferably reversible phase change, 3. crosslinkability.

In some embodiments, the polymer is partially functionalised to an extent such that the functionalised polymer is capable of one or more of the following, preferably all of the following: (I) a liquid to solid phase change caused by an ionic crosslinking agent, preferably a divalent cation, more preferably Ca²⁺, (ii) adhering cells; (iii) a solid to liquid phase change caused by a chelator chelating an ionic crosslinking agent, preferably wherein the chelator is ethylenediaminetetraacetic acid (EDTA); and (iv) a liquid to solid phase change caused by photocrosslinking, preferably by combining the functionalised polymer with a photoinitiator and exposing to visible light.

In some embodiments, from about 30% to about 70%, preferably from about 40% to about 60%, more preferably from about 44% to about 60%, of the polymer is functionalised, based on the proportion of monomer units making up the polymer that have been functionalised.

In some embodiments, from about 16.0% to about 68.8%, preferably from about 19.5% to about 68.5%, more preferably from about 31% to about 58%, of the polymer is functionalised with the plurality of photocrosslinkable moieties, based on the proportion of monomer units making up the polymer that have been functionalised. For example, in the case of an alginate polymer, from about 16.0% to about 68.8%, preferably from about 19.5% to about 68.5%, more preferably from about 31% to about 58% of hydroxyl groups of the aliginate have been functionalised with the plurality of photocrosslinkable moieties.

In some embodiments, from about 1.2% to about 15%, preferably from about 1.5% to about 10.5%, more preferably from about 2% to about 9%, of the polymer is functionalised with the plurality of cell adhesion moieties, based on the proportion of monomer units making up the polymer that have been functionalised.

In some embodiments, the polymer comprises or consists of a natural polymer, preferably alginate. The polymer may have an average molecular weight of from about 5 kDa to about 1000 kDa.

The polymer may preferably be alginate. In some embodiments, the alginate has an average molecular weight of from about 68 kDa to about 780 kDa, preferably from about 200 kDa to about 400 kDa, more preferably from about 250 kDa to about 350 kDa, more preferably from about 250 kDa to about 300 kDa, more preferably from about 260 kDa to about 290 kDa, more preferably from about 265 kDa to about 285 kDa, more preferably from about 270 kDa to about 280 kDa. In some embodiments, the alginate has an average molecular weight of about 270 kDa or about 280 kDa. In some embodiments, the alginate has a M/G ratio (ratio of mannuronic acid blocks and to guluronic acid blocks) of from about 0.30 to about 2.60, preferably from about 0.40 to about 2, more preferably from about 0.50 to about 1.5, more preferably from about 0.60 to about 1.4, more preferably from about 0.64 to about 1.30.

In some embodiments, the alginate has a M/G ratio of about 0.64 or about 1.30. In some embodiments, the alginate has a molecular weight of about 270 kDa and a M/G ratio of about 1.30. In other embodiments, the alginate has a molecular weight of about 280 kDa and a M/G ratio of about 0.64.

The ratio of M/G has been found to provide optimum control of the proportion of crosslinks formed by the divalent cationic (e.g. calcium) crosslinking agent which allows effective phase change from liquid to solid state. The ratio also allows this phase optimum effectiveness of the reverse phase change from solid to liquid on addition of a chelating agent for the crosslinking agent.

In some embodiments, alginate is modified with methacrylate groups to form methacrylated alginate. In general, methacrylated alginate is synthesized by chemical modification of carboxyl or hydroxyl groups with methacrylate groups. These methacrylate groups can then be crosslinked when combined with an ionic crosslinking agent, preferably a divalent cation, more preferably Ca²⁺, or when combined with a photoinitiator and exposed to visible light. In preferred embodiments, these methacrylate groups are first cross-linked with the ionic crosslinking agent to cause a reversible liquid to solid phase change that results in a polymer that can act as a substrate on which cells can adhere and be cultured over a period of time. Advantageously, cross-linking with an ionic crosslinking agent enables the polymer to be subsequently liquified by exposing the ionically crosslinked polymer to a chelator such as EDTA for isolation of the cells therefrom (e.g., by centrifugation) or administration of the cells and the polymer (e.g., by injection) to an individual in need thereof, or for use as a bio-ink formulation for 3D bioprinting.

In some embodiments, the polymer can be photo crosslinked at site of use (e.g., after administration of a composition comprising cells and the polymer to an individual in need). In some embodiments, the plurality of photocrosslinkable moieties are capable of crosslinking when combined with a photoinitiator and exposed to visible light. In preferred embodiments, the reactive functionality capable of photocrosslinking is a methacrylate. In preferred embodiments, the plurality of photocrosslinkable moieties are a plurality of methacrylate groups.

In some embodiments, the photocrosslinkable moiety is conjugated to the polymer via the oxygen atom of an ester group. In some embodiments, the photocrosslinkable moiety is derived from a reagent for providing the photocrosslinkable moiety that has reacted with a hydroxyl group, a carboxyl group or an amine group of the polymer, preferably a hydroxyl group.

In some embodiments, the plurality of cell adhesion moieties are conjugated to alginate by carbodiimide chemistry, thiol addition, or click chemistry. Carbamates, imines or hydrazones from the reaction with adipic acid dihydrazide after periodate oxidation of alginate can also be used.

In some embodiments, the cell adhesion moiety is any peptide sequence with cysteine and/or thiol (—SH) functionality. In some embodiments, the average molecular weight of the peptide is from about 500 Da to about 2500 Da, more preferably from about 500 Da to about 600 Da, for example, 536.56 Da.

In some embodiments, the plurality of cell adhesion moieties each comprise an integrin binding motif, preferably an Arg-Gly-Asp (RGD) motif (SEQ ID NO:1). The peptide may comprise or consist of RGDS (SEQ ID NO:2), GGGGRGDSP (SEQ ID NO: 3), GRGDSP (SEQ ID NO:4), or GRGDS (SEQ ID NO:5), or an amino acid sequence with 1 or 2 amino acid insertions, deletions, substitutions (preferably conservative substitutions) or a combination thereof, typically outside the RGD motif.

In some embodiments, the plurality of cell adhesion moieties are each conjugated to the linker via a thioether bond.

In some embodiments, the linker is derived from a photocrosslinkable moiety linked to the polymer that has reacted with a peptide for cell adhesion, preferably via a thiol-Michael addition reaction. The peptide for cell adhesion may preferably comprise a cysteine residue and a RGD cell adhesion motif, for example, CRGDS (SEQ ID NO:6).

In some embodiments, the cell adhesion moieties are introduced into methacrylated alginate, for example, via a thiol-Michael addition reaction. In preferred embodiments, RGD-based peptide sequences are introduced into methacrylated alginate via a thiol-Michael addition reaction to produce methacrylated alginate-RGD. The resultant methacrylated alginate-RGD may be cross-linked with the ionic crosslinking agent to cause a reversible liquid to solid phase change that results in a polymer that can act as a substrate on which cells can adhere and be cultured over a period of time.

In some embodiments, the polymer can act as a substrate on which cells—such as human cells, primary cells, non-transformed cells, or adherent cells as described herein—can adhere and be cultured over a period of time (for example, up to and including 7 days). Particularly preferred cells that are cells that have chondrogenic, osteogenic and/or adipogenic potential, such as cells from adipose tissue, such as adult stem cells. In particular, mesenchymal stem cells, or related precursors, or cells derived from these cells have the capacity to form molecules of the extracellular matrix, and in particular molecules required for chondrogenesis and cartilage repair and restoration. Adipose derived stem cells (ADSCs) are particularly useful where the method is to be utilised in a procedure for cartilage repair or restoration. ADSCs may obtained from a number of different fatty tissues of the human or animal body. The ADSCs may be autologous or allogeneic.

In some embodiments, the polymer is capable of a solid to liquid phase change caused by a chelator chelating an ionic crosslinking agent, preferably wherein the chelator is ethylenediaminetetraacetic acid (EDTA). For example, the polymer on which the cells have adhered to and been cultured on or in (e.g., ionic cross linked methacrylated alginate-RGD) can be treated with a chelator to cause a solid to liquid phase change forming a deliverable composition (e.g., by injection) or a composition for use as a bioink. Alternatively, the cells can be isolated from the polymer by centrifugation (e.g., at 250-300 G for 5 minutes) so that the liquified polymer remains in suspension and the cells form a pellet that can be recovered. The cells can then be recovered and resuspended and/or frozen prior to use. The cells may be resuspended (e.g., in a hydrogel) prior to use.

In some embodiments, the polymer is capable of photocrosslinking, preferably by combining the polymer with a photoinitiator and exposing to visible light. For example, the polymer can be photocrosslinked on administration of a composition comprising cells and the polymer to an individual in need thereof.

In some embodiments, the method further comprises the step of cross-linking the functionalised polymer by reacting the functionalised polymer with an ionic crosslinking agent to cause a reversible liquid to solid phase change of the polymer. In preferred embodiments, the step of cross-linking the functionalised polymer with the ionic crosslinking agent (i.e., to produce an ionic-cross-linked polymer) precedes any step to photo cross link the polymer, by for example, combining the polymer with a photo initiator and exposing it to visible light.

The invention provides in one set of embodiments a functionalised alginate composition comprising alginate polymer partially functionalised with:
a plurality of photocrosslinkable moieties linked to the polymer in uncrosslinked form, each photocrosslinkable moiety comprising a reactive functionality capable of photocrosslinking; and
a plurality of cell adhesion moieties, optionally associated with adhered cells, each of adhesion moieties linked to the polymer via a linker, each cell adhesion moiety comprising a cell adhesion motif;
   wherein the alginate polymer is crosslinked with a divalent crosslinking agent, such as calcium.

The photocrosslinking moieties may, for example, comprise one or more of acrolyl, methacrolyl, acrylate, methacrylate, acrylamide, methacrylamide, allyl ether, maleimide, vinyl sulfone, NHS ester and vinyl ether.

The cell adhesion moieties may, for example, selected from the group consisting of RGD (SEQ ID NO:1), RGDS (SEQ ID NO:2), GGGGRGDSP (SEQ ID NO: 3), GRGDSP (SEQ ID NO:4), or GRGDS (SEQ ID NO:5).

In some embodiments, the functionalised alginate composition comprises cells adhered to the cell adhesion moieties.

The cells, when present in the functionalised alginate composition may, for example, be cells that have chondrogenic, osteogenic and/or adipogenic potential. More specific examples include adult stem cells preferably selected from mesenchymal stem cells and adipose derived stem cells (ADSCs)

The invention further relates to the functionalised alginate composition prepared by photocrosslinking of the functionalised alginate composition optionally in the presence of a photoinitiator.

In some embodiments, the alginate polymer has an M/G ratio in the range of about 0.30 to about 2.60.

In some embodiments, the alginate component of the functionalised alginate has an average molecular weight of from about 68 kDa to about 780 kDa.

The inventors have found that the functionalised alginate polymers are capable of (a) undergoing reversible crosslinking by ionic crosslinking (e.g., exposure to divalent cations such as Ca²⁺) while presenting suitable concentrations of cell adhesion motifs, (b) liquifying by exposing the ionically crosslinked polymer to a chelator such as EDTA, and (c) undergoing irreversible photocrosslinking of photocrosslinkable groups (e.g., by adding a photoinitiator and exposing to light).

Accordingly, the invention provides a method of forming a solid composition of the polymer comprising:
(a) subjecting the alginate polymer to reversible crosslinking by ionic crosslinking (e.g., exposure to divalent cations such as Ca2+) while presenting suitable concentrations of cell adhesion motifs optionally with attached cells,
(b) liquifying the alginate polymer by exposing the ionically crosslinked polymer to a chelator such as EDTA, and
(c) undergoing irreversible photocrosslinking of photocrosslinkable groups (e.g., by adding a photoinitiator and exposing to light).

The steps of phase transfer from solid to liquid and then from liquid to solid are particularly advantageous in allowing culturing of cells in association with a solid network and to then transform the alginate composition to a liquid form for placement of the cell culture (e.g., delivery in vitro or in vivo) or isolation of the cells from the polymer (e.g., by centrifugation).

Accordingly, the invention provides method of cell culture comprising:
providing an alginate polymer composition comprising an alginate polymer partially functionalised with:
a plurality of photocrosslinkable moieties linked to the polymer in uncrosslinked form, each photocrosslinkable moiety comprising a reactive functionality capable of photocrosslinking; and
a plurality of cells bound to the alginate polymer by adhesion moieties each linked to the polymer via a linker, each cell adhesion moiety comprising a cell adhesion motif;
wherein the alginate polymer is crosslinked by a divalent cationic crosslinking agent, such as calcium, to form a solid alginate polymer
culturing the cells bound to the crosslinked alginate polymer under conditions and for a time that allow or causes the cell number to increase;
optionally, liquifying the ionic crosslinked alginate polymer by addition of a chelating agent, such as EDTA, for the cationic crosslinking agent to form a liquified alginate polymer composition comprising cells.
optionally, applying the liquified alginate polymer composition comprising cells to a substrate (e.g., administering the liquified alginate polymer comprising cells to an injury site); and
optionally, photopolymerising the liquified alginate polymer composition comprising cells to cause the photocrosslinkable moieties of the alginate polymer to photocrosslink the alginate polymer and form a solid cross-linked structure comprising the cells.

The alginate polymer composition may be formed by a method comprising:
(a) providing an alginate composition comprising alginate polymer partially functionalised with:
   a plurality of photocrosslinkable moieties linked to the polymer, each photocrosslinkable moiety comprising a reactive functionality capable of photocrosslinking; and
   a plurality of cell adhesion moieties each linked to the polymer via a linker, each cell adhesion moiety comprising a cell adhesion motif;
(b) binding cells to the plurality of cell adhesion moieties linked to the alginate polymer; and
(c) adding a divalent cationic crosslinking agent, such as calcium ions, to crosslink the alginate polymer comprising bound cells to induce a phase change of the alginate polymer from liquid to solid.

Alternatively, the alginate polymer composition may be formed by a method comprising:
(a) providing an alginate composition comprising alginate polymer partially functionalised with:
   a plurality of photocrosslinkable moieties linked to the polymer, each photocrosslinkable moiety comprising a reactive functionality capable of photocrosslinking; and
   a plurality of cell adhesion moieties each linked to the polymer via a linker, each cell adhesion moiety comprising a cell adhesion motif;
(b) adding a divalent cationic crosslinking agent, such as calcium ions, to crosslink the alginate polymer to induce a phase change of the alginate polymer from liquid to solid; and
(c) binding cells to the plurality of cell adhesion moieties linked to the crosslinked alginate polymer having a solid crosslinked structure.

The photopolymerising of the liquified alginate may be carried out in the presence of a photoinitiator which may be added to the liquified alginate polymer comprising cells prior to photoinitiation, such as following liquification and prior to photopolymerisation.

The cell culture may be applied to the substrate by, for example, injection, extrusion or 3D printing. Typically the consistency of the liquified alginate will allow application via injection.

In some embodiments, the cell culture is for administration to an articular surface requiring repair or restoration.

In one set of embodiments, the cell culture is for administration during open surgery.

In another aspect, the present invention provides a method for preparing the functionalised polymer described herein, the method comprising:
providing a polymer partially functionalised with a plurality of photocrosslinkable moieties, each photocrosslinkable moiety comprising a reactive functionality capable of photocrosslinking; and
reacting a peptide for cell adhesion with a portion of the plurality of the photocrosslinkable moieties present on the polymer, to thereby provide the plurality of cell adhesion moieties linked to the polymer via a linker;
   thereby providing the functionalised polymer.

In some embodiments, the method further comprises the step of cross-linking the functionalised polymer by reacting the functionalised polymer with an ionic crosslinking agent to cause a reversible liquid to solid phase change of the polymer. In preferred embodiments, the step of cross-linking the functionalised polymer with the ionic crosslinking agent (i.e., to produce an ionic-cross-linked polymer) precedes any step to photo cross link the polymer, by for example, combining the polymer with a photo initiator and exposing it to visible light.

In another aspect, the present invention provides a method for preparing the functionalised polymer described herein, the method comprising:
providing a polymer comprising a plurality of functional groups;
reacting a reagent for providing a photocrosslinkable moiety with a portion of the plurality of functional groups present on the polymer, to provide a polymer partially functionalised with a plurality of photocrosslinkable moieties; and
reacting a peptide for cell adhesion with a portion of the plurality of photocrosslinkable moieties present on the polymer, to thereby provide a plurality of cell adhesion moieties linked to the polymer via a linker;
   thereby providing the functionalised polymer.

In some embodiments, the method further comprises the step of cross-linking the functionalised polymer by reacting the functionalised polymer with an ionic crosslinking agent to cause a reversible liquid to solid phase change of the polymer. In preferred embodiments, the step of cross-linking the functionalised polymer with the ionic crosslinking agent (i.e., to produce an ionic-crosslinked polymer) precedes any step to photo cross link the polymer, by for example, combining the polymer with a photo initiator and exposing it to visible light.

In some embodiments, the step of reacting a peptide for cell adhesion comprises reacting the peptide for cell adhesion, which preferably comprises a cysteine residue, with a portion of the plurality of photocrosslinkable moieties present on the polymer via a thiol-Michael addition reaction.

In some embodiments, the polymer partially functionalised with a plurality of photocrosslinkable moieties is from about 30% to about 70%, preferably from about 40% to about 60%, functionalised with the plurality of photocrosslinkable moieties.

In some embodiments, the step of reacting a reagent for providing a photocrosslinkable moiety comprises reacting the reagent with from about 30% to about 70%, preferably from about 40% to about 60%, of the plurality of functional groups present on the polymer.

In some embodiments, the step of reacting a peptide for cell adhesion comprises reacting the peptide for cell adhesion with from about 4% to about 20%, preferably from about 5% to about 15%, more preferably from about 5% to about 10%, of the photocrosslinkable moieties present on the polymer.

In preferred embodiments, the polymer partially functionalised with a plurality of photocrosslinkable moieties is alginate partially functionalised with methacrylate. In preferred embodiments, the peptide for cell adhesion comprises a cysteine residue and a RGD cell adhesion motif.

In another aspect, the present invention provides the functional polymer prepared by a method described herein.

In another aspect, the invention provides a polymer composition comprising the functionalised polymer described herein or prepared by a method of the invention herein, and an aqueous solution.

In some embodiments, the aqueous solution is a buffer solution, for example phosphate buffered saline (PBS), triethanolamine (TEOA) buffered saline, (4-(2-hydroxyethyl)-1-piperazineethanesulfonic acid) (HEPES) buffered saline or a cell culture medium such as Dulbecco's Modified Eagle Medium (DMEM).

In some embodiments, the polymer composition comprises the functionalised polymer in an amount of from about 3% w/v to about 10% w/v, preferably from about 5% w/v to about 8% w/v, based on the volume of aqueous solution.

Preferably, the polymer composition is capable of forming a hydrogel having the following properties: 1. cellular adhesion, 2. inducible phase change, preferably reversible phase change, 3. crosslinkability.

In another aspect, the invention provides a method for forming a liquified polymer composition comprising cells from a tissue sample, the method comprising:
providing a tissue sample comprising cells;
contacting the sample with the functionalised polymer described herein, or prepared by the method of the invention described herein, or the polymer composition described herein, in binding conditions, said binding conditions being conditions that enable binding of cells in the sample to the functionalised polymer, so that said cells are bound to the functionalised polymer;
culturing the cells bound to the polymer under conditions and for a time that allows, or causes the cell number to increase;
providing conditions to induce a solid to liquid phase change of the functionalised polymer;
   thereby forming a liquified polymer composition comprising cells from a tissue sample.

In another aspect, the invention provides a method for forming a liquified polymer composition comprising cells from a tissue sample, the method comprising:
providing cells isolated from a tissue sample;
contacting the cells with the functionalised polymer described herein, or prepared by the method of the invention described herein, or the polymer composition described herein, in binding conditions, said binding conditions being conditions that enable binding of the cells to the functionalised polymer, so that said cells are bound to the functionalised polymer;
culturing the cells bound to the polymer under conditions and for a time that allows, or causes, the cell number to increase;
providing conditions to induce a solid to liquid phase change of the functionalised polymer;
   thereby forming a liquified polymer composition comprising cells from a tissue sample.

In another aspect, the invention provides a method for forming a liquified polymer composition comprising cells from a tissue sample, the method comprising:
providing cells bound to the functionalised polymer described herein, or prepared by the method of the invention described herein, or the polymer composition described herein;
culturing the cells bound to the polymer under conditions and for a time that allows, or causes, the cell number to increase;
providing conditions to induce a solid to liquid phase change of the functionalised polymer;
   thereby forming a liquified polymer composition comprising cells from a tissue sample.

In another aspect, the invention provides a method for forming a liquified polymer composition comprising cells having chondrogenic potential from a tissue sample (e.g., an adipose tissue sample), the method comprising:
providing a tissue sample (e.g., an adipose tissue sample) comprising cells having chondrogenic potential;
contacting the sample with the functionalised polymer described herein, or prepared by the method of the invention described herein, or the polymer composition described herein, in binding conditions, said binding conditions being conditions that enable binding of cells having chondrogenic potential in the sample to the functionalised polymer, so that said cells are bound to the functionalised polymer;
culturing the cells bound to the functionalised polymer under conditions and for a time that allows, or causes, the cell number to increase;
providing conditions to induce a solid to liquid phase change of the functionalised polymer;
   thereby forming a liquified polymer composition comprising cells having chondrogenic potential from a tissue sample.

In another aspect, the invention provides a method for forming a composition comprising cells having chondrogenic potential from a tissue sample (e.g., an adipose tissue sample), the method comprising:
providing a tissue sample comprising cells having chondrogenic potential;
isolating the cells from the extracellular matrix in the tissue sample;
contacting the isolated cells with the functionalised polymer described herein, herein or prepared by the method of the invention described herein, or the polymer composition described herein, in binding conditions, said binding conditions being conditions that enable binding of cells having chondrogenic potential to the functionalised polymer, so that said cells are bound to the functionalised polymer;
culturing the cells bound to the functionalised polymer under conditions and for a time that allows, or causes, the cell number to increase;
providing conditions to induce a solid to liquid phase change of the functionalised polymer;
   thereby forming a liquified polymer composition comprising cells having chondrogenic potential from a tissue sample.

In another aspect, the invention provides a method for forming a liquified polymer composition comprising cells having chondrogenic potential from a tissue sample (e.g., an adipose tissue sample), the method comprising:
providing a tissue sample (e.g., an adipose tissue sample) comprising cells having chondrogenic potential;
isolating the cells from the extracellular matrix in the tissue sample;
separating the isolated cells from substantially all the fat and/or liquid present in the tissue sample;
contacting the separated cells with the functionalised polymer described herein, or prepared by the method of the invention described herein, or the polymer composition described herein, in binding conditions, said binding conditions being conditions that enable binding of cells having chondrogenic potential to the functionalised polymer, so that said cells are bound to the functionalised polymer;
culturing the cells bound to the functionalised polymer under conditions and for a time that allows, or causes, the cell number to increase;
providing conditions to induce a solid to liquid phase change of the functionalised polymer;
   thereby forming a liquified polymer composition comprising cells having chondrogenic potential from a tissue sample.

In some embodiments of methods of the invention, the functionalised polymer is in a solid state prior to being contacted with the cells. In other embodiments, the functionalised polymer is in a liquid state when contacted with the cells and the method subsequently provides conditions that induce a liquid to solid state change of the polymer (e.g., by ionic cross-linking).

In some embodiments of methods of the invention, the cells are contacted with an ionic cross-linked functionalised polymer having a solid cross-linked structure. In some embodiments, the ionic crosslinked functionalised polymer while capable of being photocrosslinked is not photocrosslinked.

In some embodiments of methods of the invention, the conditions to induce a solid to liquid phase change of the functionalised polymer comprise contacting the polymer with a chelator to cause a solid to liquid phase change. Advantageously, this may allow for subsequent isolation of the cells from the polymer or administration of the resulting liquified polymer composition comprising cells (e.g., by injection) to an individual in need thereof, or use as a bio-ink formulation for 3D bioprinting.

The cells may remain bound to, or retained in or on, or encapsulated by the phase changed polymer after the polymer has been phase changed from a solid to liquid.

In some embodiments, the methods of the invention further comprise the step of isolating the cells from the phase changed polymer (e.g., by centrifugation).

In another aspect, the invention provides a method for treating an individual, the method comprising:
harvesting a tissue sample (e.g., an adipose tissue sample) from an individual, or being provided with a harvested tissue sample (e.g., an adipose tissue sample) from an individual;
contacting the sample with the functionalised polymer described herein, or prepared by a method described herein, or the polymer composition described herein, in binding conditions, said binding conditions being conditions that enable binding of cells in the sample to the functionalised polymer, so that said cells are bound to the functionalised polymer;
culturing the cells bound to the polymer under conditions and for a time that allows, or causes, the cell number to increase;
providing conditions to induce a solid to liquid phase change of the functionalised polymer, thereby forming a liquified polymer composition comprising cells;
administering the liquified polymer composition, or a subsequent 3D bio printed tissue or organ to the individual;
   thereby treating the individual.

In another aspect, the invention provides a method for treating an articular cartilage defect in an individual, the method comprising:
harvesting a tissue sample (e.g., an adipose tissue sample) from an individual, or being provided with a harvested tissue sample from an individual (e.g., an adipose tissue sample), said sample comprising cells having chondrogenic potential;
contacting the sample with the functionalised polymer described herein, or prepared by a method described herein, or the polymer composition described herein, in binding conditions, said binding conditions being conditions that enable binding of cells having chondrogenic potential in the sample to the functionalised polymer, so that said cells are bound to the functionalised polymer;
culturing the cells bound to the polymer under conditions and for a time that allows, or causes, the cell number to increase;
providing conditions to induce a solid to liquid phase change of the functionalised polymer, thereby forming a liquified polymer composition comprising cells having chondrogenic potential;
administering the liquified polymer composition, or a subsequent 3D bio printed tissue, to an articular cartilage defect in the individual;
   thereby treating the articular cartilage defect in an individual.

In another aspect, the invention provides a method for treating an articular cartilage defect in an individual, the method comprising:
harvesting a tissue sample (e.g., an adipose tissue sample) from an individual, or being provided with a harvested tissue sample (e.g., an adipose tissue sample) from an individual, said sample comprising cells having chondrogenic potential;
isolating cells from the extracellular matrix in the tissue sample;
contacting the isolated cells with the polymer described herein in binding conditions, said binding conditions being conditions that enable binding of cells having chondrogenic potential in the sample to the functionalised polymer, so that said cells are bound to the functionalised polymer;
culturing the cells bound to the functionalised polymer under conditions and for a time that allows, or causes, the cell number to increase;
providing conditions to induce a solid to liquid phase change of the functionalised polymer, thereby forming a liquified polymer composition comprising cells having chondrogenic potential;
administering the liquified polymer composition, or a subsequent 3D bio printed tissue, to an articular cartilage defect in the individual;
   thereby treating the articular cartilage defect in an individual.

In another aspect, the present invention provides a method for treating an articular cartilage defect in an individual, the method comprising:
harvesting a tissue sample (e.g., an adipose tissue sample) from an individual, or being provided with a harvested tissue sample (e.g., an adipose tissue sample) from an individual, said sample comprising cells having chondrogenic potential;
isolating cells from the extracellular matrix in the tissue sample;
separating the isolated cells from substantially all the fat and/or liquid present in the tissue sample;
contacting the sample with the functionalised polymer described herein in binding conditions, said binding conditions being conditions that enable binding of cells having chondrogenic potential in the sample to the functionalised polymer, so that said cells are bound to the functionalised polymer;
culturing the cells bound to the polymer under conditions and for a time that allows, or causes, the cell number to increase;
providing conditions to induce a solid to liquid phase change of the functionalised polymer, thereby forming a liquified polymer composition comprising cells having chondrogenic potential;
administering the liquified polymer composition, or a subsequent 3D bio printed tissue to an articular cartilage defect in the individual;
   thereby treating the articular cartilage defect in the individual.

In some embodiments of methods of the invention, the functionalised polymer is in a solid state prior to being contacted with the cells. In other embodiments, the functionalised polymer is in a liquid state when contacted with the cells and the method subsequently provides conditions that induce a liquid to solid state of the polymer.

In some embodiments of methods of the invention, the cells are contacted with an ionic cross-linked functionalised polymer having a solid cross-linked structure. In some embodiments, the ionic crosslinked functionalised polymer while capable of being photocrosslinked is not photocrosslinked.

In some embodiments of methods of the invention, the conditions to induce a solid to liquid phase change of the functionalised polymer comprise contacting the polymer with a chelator to cause a solid to liquid phase change. Advantageously, this may allow for subsequent isolation of the cells from the polymer or administration of the resulting liquified polymer composition comprising cells (e.g., by injection) to an individual in need thereof, or use as a bio-ink formulation for 3D bioprinting.

The cells may remain bound to, or retained in or on, or encapsulated by the phase changed polymer after the polymer has been phase changed from a solid to liquid.

In particularly preferred embodiments of methods of the invention, the liquified polymer composition is photo crosslinked after administration to the individual to be treated by combining the liquified polymer composition with a photo initiator prior to administration and exposing the composition to UV light after administration to the individual to be treated.

In some embodiments, the cells are autologous (i.e., from the individual to be treated). In other embodiments, the cells are allogeneic.

In another aspect, the present invention provides a method for culturing cells, the method comprising:
providing cells;
contacting the cells with the functionalised polymer described herein, or prepared by the method of the invention described herein, or the polymer composition described herein, in binding conditions, said binding conditions being conditions that enable binding of cells in the sample to the functionalised polymer, so that said cells are bound to the functionalised polymer;
culturing the cells bound to the polymer under conditions and for a time that allows, or causes, the cell number to increase;
optionally, providing conditions to induce a solid to liquid phase change of the functionalised polymer to form a liquified polymer composition comprising cells;
optionally collecting the liquified polymer composition comprising cells, for example, from a culture vessel (e.g., a culture plate or flask);
optionally isolating cells from the liquified polymer composition by, for example, centrifugation;
optionally, isolating cells from the functionalised polymer for use (in for example, treatment) or for further culture and expansion;
optionally, isolating nucleic acids (e.g., RNA), peptides, or proteins from the culture.

In some embodiments, the functionalised polymer is in a solid state prior to being contacted with the cells. In other embodiments, the functionalised polymer is in a liquid state when contacted with the cells and the method subsequently provides conditions that induce a liquid to solid state of the polymer.

In some embodiments, the cells are contacted with an ionic cross-linked functionalised polymer having a solid cross-linked structure. In some embodiments, the ionic crosslinked functionalised polymer while capable of being photocrosslinked is not photocrosslinked. In some embodiments, the ionic cross-linked functionalised polymer has a thickness of about 0.5 to about 1 mm.

In some embodiments, the conditions to induce a solid to liquid phase change of the functionalised polymer comprise contacting the polymer with a chelator to cause a solid to liquid phase change.

In some embodiments of the present invention, the cells may remain bound to, or retained in or on, or encapsulated by the phase changed polymer.

In another aspect, the present invention provides a polymer composition comprising cells, preferably a composition formed, obtained or obtainable by a method of the invention as described herein, preferably wherein the composition comprises cells having chondrogenic potential, preferably wherein the functionalised polymer comprises the following features: 1. cellular adhesion, 2. inducible phase change, preferably reversible phase change, and 3. crosslinkability. Preferably, the composition does not comprise fibroblasts.

In another aspect, the present invention provides a use of a composition formed by a method of the invention as described herein, or a composition of the invention as described herein, in the manufacture of a medicament for treatment of a condition requiring re-implantation of cells for said treatment, for example, to treat an articular cartilage defect.

In another aspect, the present invention provides a composition formed by a method of the invention described herein, or a composition of the invention as described herein, for use in the treatment of a condition requiring implantation of cells for said treatment.

In another aspect, the present invention provides a composition formed by a method of the invention as described herein, or a composition of the invention as described herein, when used for treatment of a condition requiring implantation of cells for said treatment.

In another aspect, the present invention provides a method of treatment comprising administering a composition formed by a method of the invention as described herein, or a composition of the invention as described herein, to an individual in whom said treatment is required.

In another aspect, the present invention provides a device or apparatus adapted for use in a method of the invention as described herein.

In another aspect, the present invention provides a kit for use, or when used, in a method of the invention, the kit comprising the functionalised polymer described herein or prepared by a method of the invention described herein or a polymer composition described herein. Preferably, the kit further comprises written instructions to perform a method of the invention described herein.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Schematic illustrating ionic crosslinking and photo crosslinking properties of the polymer described herein.

FIG. 2. Graphs showing photorheology and storage modulus on calcium crosslinking of the universal polymer (Alg-MA-RGD) and its precursor alginate methacrylate (Alg-MA). (a), (b) show the photorheology and storage modulus on cationic crosslinking respectively of Alg-MA with 40% methacrylate DOF (black squares) and Alg-MA-RGD prepared from Alg-MA having 40% DOF with 5% RGD DOF (red circles). (c), (d) show the photorheology and storage modulus on cationic crosslinking respectively of Alg-MA with 60% methacrylate DOF (black squares), and Alg-MA-RGD prepared from Alg-MA having 60% DOF with 5% RGD DOF (red circles) or 15% RGD DOF (blue triangles).

FIG. 3. Graph showing metabolic activity of human adipose derived mesenchymal stem cells (hADCS), isolated from infrapatellar fat pad of a donor patient, seeded at 1.6×10⁵ cell/cm² s on 15×1 mm discs of CaCl₂) crosslinked Alg-MA-RGD formulations (Formulation 1: blue solid line, Formulation 2: blue dashed line, Formulation 3: red solid line, Formulation 4: red dashed line, Formulation 5: green solid line, Formulation 6: green dashed line) and a tissue culture plate plastic control (black solid line). The metabolic activity was quantified with CellTiterBlue assay at the days 1, 3 and 7 and plotted as fold change.

FIG. 4. Images of cells at human adipose derived mesenchymal stem cells (hADCS), isolated from infrapatellar fat pad of a donor patient, seeded at 1.6×10⁵ cell/cm² s on 15×1 mm discs of CaCl₂) crosslinked Formulation 6 at days 1, 3 and 7 of metabolic activity assay.

FIG. 5. Photographs of photocrosslinked samples of Alg-MA-RGD formulations Formulation 2 (a), Formulation 4 (b) and Formulation 6 (c) compared to photocrosslinked GelMA 6% (d).

FIG. 6. Graphs showing metabolic activity and compressive modulus of photocrosslinked Alg-MA-RGD formulations. (a) shows metabolic activity hADCS isolated from infrapatellar fat pad of a donor patient, seeded at 1.6×10⁵ cell/cm² on 8×2 mm discs of calcium crosslinked Alg-MA-RGD formulations (Formulations 1-6). Phase change was performed at day 7 using 60 μL of 250 mM EDTA, the bioink was mixed with lithium phenyl-2,4,6-trimethylbenzoylphosphinate (LAP) at a final concentration of 0.1% and photocrosslinking induced with 405 nm wavelength at 20 mW/cm² for 60 seconds. Tissue culture plate plastic was used as a control: cells were seeded at 1.6×10⁵ cell/cm², detach with trypsin/EDTA at day 7, mixed with gelatin methacrylate (GelMA) 6% and 0.1% LAP and photocrosslinking induced with 405 nm wavelength at 20 mW/cm² for 60 seconds. Metabolic activity was quantified with a CellTiterBlue assay 24 hours after delivery and plotted as percentage of metabolic activity (percentage over GelMA control). (b) shows compressive modulus (10-15% strain) of Alg-MA-RGD formulations (Formulations 1, 3 and 5) and GelMA control, generated after phase change with EDTA and photocrosslinked at 20 mW/cm² at 405 nm for 60 seconds in 4×2 mm cylindrical moulds.

FIG. 7. Panel A. Representative images of the entire bioscaffolds for the indicated batches. The first line show the material only scaffolds with no cells, while the second and third rows show the bioscaffolds under 21 days cultivation with cell culture growing media (CTRL) and chondrogenic differentiation media (CHONDRO). Panel B. (I) Representative brightfield image showing bioscaffold prepared from batch #446 at the start of the chondrogenic process, acquired with EVOS FL (4× objective 0.13 NA), scale bar=1 mm. The close-up image showing immunohistological analysis of a cryosection of the scaffold stained with DAPI (nuclei, white) and anti-Collagen Type II (#II6B3, Cyan), acquired with a NIKON A1R confocal microscope (20× objective 0.75 NA), scale bar=200 μm. (II) Graph showing fold changes of Glycosaminoglycans, and DNA extracted from the scaffolds using Papain Buffer and quantified respectively using DMMB assay (Glycosaminoglycans) and Quant-IT PicoGreen assay (DNA). (III) Phase contrast image showing bioscaffold from batch #446 at the end of the chondrogenic process, acquired with EVOS FL (4× objective 0.13 NA), scale bar=1 mm. The close-up images show immunohistological analysis of three different areas of a cryosection of the scaffold stained with DAPI (nuclei, white) and anti-Collagen Type II (#II6B3, Cyan), acquired with a NIKON A1R confocal microscope (20× objective 0.75 NA), scale bar=200 μm.

FIG. 8. Graphs showing storage modulus of calcium crosslinked (solid bars) and photocrosslinked (shaded bars) blends of alginate, Alg-MA and Alg-RGD. Alg-RGD content was kept at 20% for all mixtures while the alginate and Alg-MA were varied from 10% to 70% respectively.

FIG. 9. Compressive modulus of Universal Polymer at Day 1 and Day 7 under different concentration of CaCO₃ ionic crosslinker.

FIG. 10. Decrosslinking rate showing that EDTA at the indicated concentration. 83 mM is the optimal concentration to decrosslink the samples crosslinked with all the CaCl₂ concentrations under 30 minutes of exposure at 37° C. A) Time to decrosslink the samples against the concentration of the chemical crosslinker CaCO₃. B) Table containing all the values for timing and concentration.

FIG. 11. Storage modulus of the Universal Polymer samples decrosslinked with EDTA at different concentrations and then exposed to 405 nm light (UV ON), showing that the increase in the concentration of the chemical crosslinker is proportional to the increase in the final storage modulus of the photocrosslinked samples.

FIG. 12. Human Adipose derived mesenchymal stem cells (hADSC), isolated from infrapatellar fat pad of donor patient [HREC/16/SVHM/186], were seeded at 1×10⁵ cells on top of CaCO3 crosslinked Universal Polymer layer and a tissue culture plate plastic control.

FIG. 13. Human Adipose derived mesenchymal stem cells (hADCS), isolated from infrapatellar fat pad of donor patient [HREC/16/SVHM/186], were seeded at 1×10⁵ cells on top of CaCO₃ crosslinked Universal Polymer layer (indicated in the legend on the top of the graph) and a tissue culture plate plastic control. The metabolic activity was quantified with CellTiterBlue assay [Promega G808] at the indicated time points and fold changes are reported.

FIG. 14. Human Adipose derived mesenchymal stem cells (hADCS), isolated from infrapatellar fat pad of donor patient [HREC/16/SVHM/186], were seeded at 1×10⁵ cell, on top of 100 mM CaCO₃ crosslinked layer of Universal polymer, let them grow for 7 days and then phase changed with the three decrosslinkers agents (x axis). The phase change for the universal polymer was performed at day 7 using 40 ul of decrosslinkers. The metabolic activity was quantified for all the conditions with CellTiterBlue assay [Promega G808] and percentages calculated based on cells number at DAY7 before phase change (100%).

FIG. 15. The bio-ink (liquified phase changed Universal Polymer+cells) was mixed with LAP at a final concentration of 0.1% w/v and bioscaffolds photocrosslinked with 405 nm wavelength at 20 mW/cm² for 60 sec. Non-irradiated samples were used as a control (−Light). The metabolic activity was quantified for all the conditions with CellTiterBlue assay [Promega G808].

FIG. 16. Total RNA from cellular scaffolds were harvested after 7 days of chondrogenic induction using Tri Reagent (Ambion, Austin, TX, USA) according to the manufacturer's protocol. Total RNA was purified using DirectZol RNA kit (Zymoresearch, CA, USA) following manufacturer's instructions including DNAse I treatment at 6 U/μl for 15 minutes. 120 ng of RNA were reverse transcribed using High-Capacity cDNA Reverse Transcription Kit (Thermo Scientific) following the manufacturer's protocol. The relative amounts of SOX9 (as target gene) and GAPDH (as housekeeping gene) were evaluated with TaqMan Gene expression assay (Applied Biosystems, Foster City, CA, USA) using the following probes: SOX9 (Hs00165814_m1) and GAPDH (Hs02786624_g1). qPCR was performed on a QuantStudio 6 Flex Real-Time PCR System (Thermo Fisher Scientific) and relative quantification was calculated with the 2e-ΔΔCT method.

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For the purposes of interpreting this specification, terms used in the singular will also include the plural and vice versa.

Unless defined otherwise, all technical and scientific terms used herein have the same meaning as commonly understood by those of ordinary skill in the art to which the present disclosure belongs. Although any methods and materials similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e. to at least one) of the grammatical object of the article. By way of example, “a reactive functionality” means one reactive functionality or more than one reactive functionality.

As used herein, the term “and/or”, e.g., “X and/or Y” will be understood to mean either “X and Y” or “X or Y” and shall be taken to provide explicit support for both meanings or for either meaning.

As used herein, the term “about” refers to a quantity, value, dimension, size, or amount that varies by as much as 10%, 5%, 1% or 0.1% to a reference quantity, value, dimension, size, or amount.

Throughout the present disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein, unless the context requires otherwise, the term “comprise”, and variations such as “comprises” and “comprising”, will be understood to imply the inclusion of a stated integer or step or group of integers or steps but not the exclusion of any other integer or step or group of integers or steps.

A “subject” herein is preferably a human subject. It will be understood that the terms “subject” and “individual” are interchangeable in relation to an individual requiring administration of the aqueous formulation of the present disclosure.

The reference to average molecular weight is to weight average molecular weight.

The shear storage modulus of Alg-MA-RGD 5% w/v (aq) was measured using oscillatory rheology with cone-plate geometry (15 mm cone with 1 deg cone angle) at a strain of 1%, a frequency of 10 rad/s, and a temperature of 23 C. The sample volume is dictated by the measuring geometry and in this case is 22 uL.

For ionic crosslinking: After 60 s of measurement time, the divalent cationic crosslinking agent, particularly CaCl₂ solution (1M concentration) was added in excess (1.5 ml) around the alginate sample using a dropper, so that the alginate is completely Immersed. The storage modulus increased with time as the divalent cations (particularly Ca2+ ions) diffused into the alginate sample.

For photo-crosslinking: The 5% w/v (aq) solution of Alg-MA-RGD was made up to include 0.1% of the water soluble photoinitiator Lithium phenyl-2,4,6-trimethylbenzoylphosphinate (LAP). After 100 s of measurement time, the sample was exposed to 405 nm light at an intensity of 20 mW/cm². The storage modulus increased with time due to the photoinitiated crosslinking of the methacrylate groups.

It will be understood that the invention disclosed and defined in this specification extends to all alternative combinations of two or more of the individual features mentioned or evident from the text. All of these different combinations constitute various alternative aspects of the invention.

The present invention relates to functionalised polymers which are capable of functioning in a sequential process as a cell culture substrate, a bio-ink and a bio-scaffold for tissue engineering applications. The present invention mitigates or entirely removes one or more of the problems in the prior art. A key aspect of the invention is the use of a single ‘universal’ polymer for all of the steps of the process (isolation, purification, expansion, detachment and/or delivery).

The present invention relates to the use of functionalised biopolymer compositions which have capability to (i) isolate a desired cell population from a stromal mass by way of contact, (ii) provide a substrate for continued culture and/or proliferation of the desired population (while having a stiffness similar to that of native tissues) (iii) liquefy in a manner that causes encapsulation of cells within the material (obviating the need for harsh detachment treatments), (iv) subsequent delivery by means of injection or as a bio-ink formulation for 3D bioprinting.

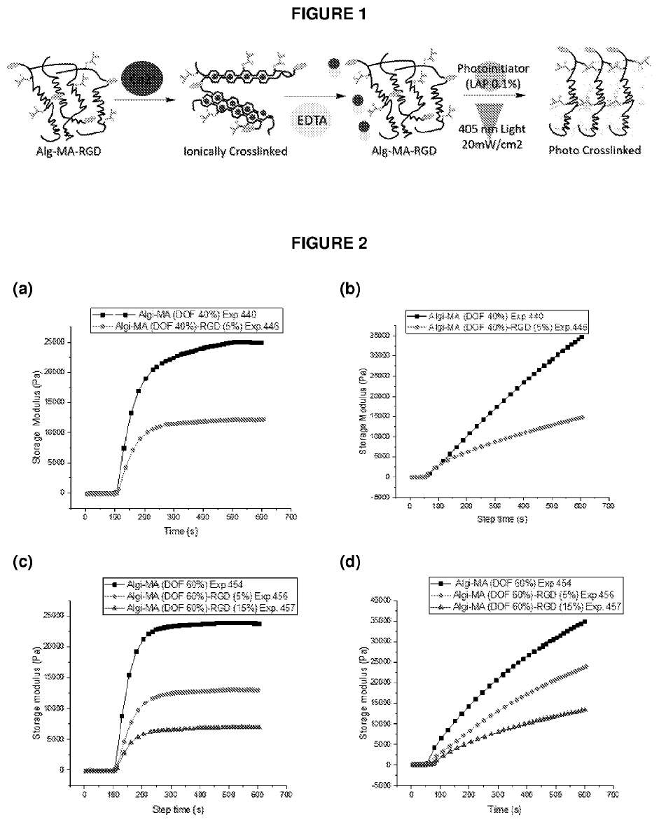

An advantage of the present invention is that it involves the use of a single polymer composition as the biomaterial environment for isolation of cells, cell culture or expansion and surgical implantation. Specifically, the inventors have found that the functionalised polymers are capable of (a) undergoing reversible crosslinking by ionic crosslinking (e.g., exposure to divalent cations such as Ca²⁺) while presenting suitable concentrations of cell adhesion motifs, (b) liquifying by exposing the ionically crosslinked polymer to a chelator such as EDTA, and (c) undergoing irreversible photocrosslinking of photocrosslinkable groups (e.g., by adding a photoinitiator and exposing to light), as shown in the schematic in FIG. 1. These properties advantageously allow the functionalised polymer of the invention to (a) function as a cell culture substrate for proliferation of cells, (b) enable cell detachment (without trypsin) by undergoing phase change to a liquid and function as a bio-ink for bioprinting in situ (the cells encapsulated by, or contained within, the liquefied polymer), and (c) provide a suitable 3D environment for tissue engineering applications such as chondrogenesis for cartilage repair.

The inventors have also found that polymer substrates can be utilised to generate a clinically useful number of purified cells for (re) implantation. A further advantage of the process is that the extraction of cells from the harvested tissue directly into the substrate supports the viability of the cells prior to and after (re) implantation. This avoids the need for processing or formulation of cells post extraction and prior to (re) implantation.

Still further, the inventors have found that the re-implanted cells are functional. For example, with cells having chondrogenic potential, the method generates cells with the capacity to develop cartilage in damaged articular surfaces.

Some of the advantages arising from the polymer include:
Avoiding the need for animal derived enzymatic compounds such as trypsin;
Allowing cellular adaption to a substrate that will ultimately be used in re-implantation to support cells;
Mitigating the risk of dedifferentiation which can occur when autologous cells are cultured on, for example, tissue culture plastic; and/or
Allowing for the generation of a closed system which can minimise contamination risk.

The present invention avoids one or more elements of the prior art which can reduce the therapeutic capacity of the cell population.

The methods, uses and compositions of the invention, which involve the use of the functionalised polymer of the invention, may find application in the provision of cells for implementation in cell therapy and/or surgical techniques.

### Functionalised Polymer

The present invention provides a functionalised polymer, which comprises a polymer partially functionalised with:
a plurality of photocrosslinkable moieties directly linked to the polymer, each photocrosslinkable moiety comprising a reactive functionality capable of photocrosslinking; and
a plurality of cell adhesion moieties each linked to the polymer via a linker, each cell adhesion moiety comprising a cell adhesion motif.

It will be understood that before functionalisation, the polymer comprises a plurality of functional groups being capable of being functionalised (e.g. capable of being functionalised by a reagent for providing a photocrosslinkable moiety). Accordingly, ‘partially functionalised’ will be understood to mean that at least one, but not all, such functional groups present in the polymer before functionalisation have been functionalised. It will also be understood that the polymer is composed of one or more types of monomer unit, and the functional groups may be present on one or more of the types of monomer unit making up the polymer.

The functionalised polymer may preferably further comprises a plurality of functional groups capable of ionic crosslinking with an ionic crosslinking agent. The plurality of functional groups capable of ionic crosslinking may preferably be provided by (on) the polymer, that is, the functional groups may be present on one or more of the types of monomer unit making up the polymer.

Accordingly, in some embodiments, the functionalised polymer comprises a polymer comprising a plurality of functional groups capable of ionic crosslinking with an ionic crosslinking agent, the polymer partially functionalised with:
a plurality of photocrosslinkable moieties linked to the polymer, each photocrosslinkable moiety comprising a reactive functionality capable of photocrosslinking; and
a plurality of cell adhesion moieties each linked to the polymer via a linker, each cell adhesion moiety comprising a cell adhesion motif.

The plurality of functional groups capable of ionic crosslinking may comprise any functional group capable of ionic crosslinking with an ionic crosslinking agent present on a polymer. The ionic crosslinking agent may be any suitable ionic crosslinking agent known in the art, including those described herein. In some embodiments, the ionic crosslinking agent is a divalent cation, preferably Ca²⁺. Suitable functional groups capable of ionic crosslinking include hydroxyl groups, carboxyl groups and amine groups. The plurality of functional groups capable of ionic crosslinking may or may not be the same as the plurality of functional groups being capable of being functionalised (e.g. capable of being functionalised by a reagent for providing a photocrosslinkable moiety) of the polymer. In some embodiments, the functional group capable of ionic crosslinking comprises one or more of hydroxyl groups, carboxyl groups and amine groups, preferably carboxyl groups. In preferred embodiments, the plurality of functional groups capable of ionic crosslinking is a plurality of carboxyl groups. In preferred embodiments, the functional groups are carboxyl groups.

Preferably, the functionalised polymer may comprise the following features, or be suitable for forming a hydrogel comprising the following features: 1. cellular adhesion, 2. inducible phase change, preferably reversible phase change, 3. crosslinkability. In this context, ‘phase change’ will be understood to refer to a change in physical state, for example a change to a solid (including gel) or a liquid phase. These features may be achieved by the functionalised polymer, which may provide (a) a plurality of functional groups capable of ionic crosslinking, (b) a plurality of photocrosslinkable moieties which are capable of photocrosslinking, and (c) a plurality of cell adhesion moieties which are capable of adhering cells. It will be appreciated that the ability of the functionalised polymer to achieve each of these features may require a balance of (a), (b) and (c). For example, increasing the proportion of (b) may increase the extent to which a functionalised polymer can photocrosslink, but may impact the ability of the functionalised polymer to ionically crosslink and/or adhere cells due to a reduced proportion of (a) and/or (c). As another example, increasing the proportion of (c) may increase the extent to which a functionalised polymer can adhere cells, but may impact the ability of the functionalised polymer to ionically crosslink and/or photocrosslink due to a reduced proportion of (a) and/or (b).

Accordingly, in some embodiments, the polymer is partially functionalised to an extent such that the functionalised polymer is capable of a liquid to solid phase change caused by an ionic crosslinking agent, preferably a divalent cation, more preferably Ca²⁺. The polymer may preferably comprise a plurality of functional groups capable of ionic crosslinking. The polymer may be partially functionalised to an extent such that (i) the functionalised polymer comprises a sufficient amount of such functional groups to allow a liquid to solid phase change caused by ionic crosslinking and/or (ii) the ability of such functional groups to allow a liquid to solid phase change caused by ionic crosslinking is not impaired, for example due to steric hindrance by one or both of the photocrosslinkable moiety and the cell adhesion moiety on the functionalised polymer. This may advantageously allow the functionalised polymer to be suitable for use as substrate for adhering and culturing cells. Accordingly, in some embodiments, the functionalised polymer is capable of forming a solid (e.g. a crosslinked hydrogel) upon ionic crosslinking having a storage modulus which forms a stable substrate for cell culture for at least 1 day, 2 days, 3 days, 4 days, 5 days, 6 days, or 7 days, preferably at least 7 days.

The functionalised polymer may be capable of forming a solid (e.g. a crosslinked hydrogel) upon ionic crosslinking having a storage modulus of at from about 3 kPa to about 75 kPa, for example about 3 kPa, 4 kPa, 5 kPa, 6 kPa, 7 kPa, 8 kPa, 9 kPa, 10 kPa, 11 kPa, 12 kPa, 13 kPa, 14 kPa, 15 kPa, 16 kPa, 17 kPa, 18 kPa, 19 kPa, 20 kPa, 21 kPa, 22 kPa, 23 kPa, 24 kPa, 25 kPa, 26 kPa, 27 kPa, 28 kPa, 29 kPa, 30 kPa, 31 kPa, 32 kPa, 33 kPa, 34 kPa, 35 kPa, 36 kPa, 37 kPa, 38 kPa, 39 kPa, 40 kPa, 41 kPa, 42 kPa, 43 kPa, 44 kPa, 45 kPa, 46 kPa, 47 kPa, 48 kPa, 49 kPa, 50 kPa, 51 kPa, 52 kPa, 53 kPa, 54 kPa, 55 kPa, 56 kPa, 57 kPa, 58 kPa, 59 kPa, 60 kPa, 61 kPa, 62 kPa, 63 kPa, 64 kPa, 65 kPa, 66 kPa, 67 kPa, 68 kPa, 69 kPa, 70 kPa, 71 kPa, 72 kPa, 73 kPa, 74 kPa, or 75 kPa. Any minimum and maximum can be combined to form a range provided that the range is between 3 kPa to 75 kPa, such as a storage modulus of from about 5 kPa to about 50 kPa. In some embodiments, the functionalised polymer is capable of forming a solid (e.g. a hydrogel) upon ionic crosslinking having a storage modulus of at from about 5 kPa to about 50 kPa, preferably from about 10 kPa to about 50 kPa.

In some embodiments, the polymer is partially functionalised with the plurality of cell adhesion moieties to an extent such that the functionalised polymer is capable of adhering cells. The functionalised polymer may comprise a sufficient amount of cell adhesion moieties to allow cell adhesion, preferably to the functionalised polymer upon ionic crosslinking. This may advantageously allow the functionalised polymer to be suitable for use as a cell culture substrate. The functionalised polymer may be capable of exhibiting at least about 80%, 81%, 82%, 83%, 84%, 85%, 86%, 87%, 88%, 89%, 90%, 91%, 92%, 93%, 94%, 95%, 96%, 97%, 98% or 99% cell adhesion, preferably at least about 93%, 94%, 95%, 96%, 97%, 98% or 99% cell adhesion, where percent adhesion is calculated according to the following equation:

$$\% adhesion = ( \frac{Seeded Cells - Unattached Cells}{Seeded cells} ) \times 100$$

In some embodiments, the polymer is partially functionalised to an extent such that the functionalised polymer is capable of a solid to liquid phase change caused by a chelator chelating an ionic crosslinking agent, preferably wherein the chelator is ethylenediaminetetraacetic acid (EDTA). This may advantageously allow the functionalised polymer to be suitable for use as a bio-ink, which may be delivered by injection or cast to subsequently form a bioscaffold. The functionalised polymer may be capable of a solid to liquid phase change caused by a chelator chelating an ionic crosslinking agent within about 30 minutes, for example about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 minutes. Any minimum and maximum can be combined to form a range provided that the range is within 30 minutes, such as within from about 2 minutes to about 25 minutes, preferably within from about 5 minutes to about 15 minutes. The chelator may be EDTA, preferably from about 0.5 mM to about 250 mM, more preferably 250 mM EDTA, which may be added at a volume ratio of functionalised polymer to EDTA of about 3:1.

In some embodiments, the polymer is partially functionalised to an extent such that the functionalised polymer is capable of a liquid to solid phase change caused by photocrosslinking, preferably by combining the functionalised polymer with a photoinitiator and exposing to visible light. The functionalised polymer may comprise a sufficient amount of photocrosslinkable moieties to allow a liquid to solid phase change, for example to form a gel. This may advantageously allow the functionalised polymer to be suitable for use as a bioscaffold, which may be capable of retaining shape after implantation, retaining cell viability and having storage modulus after photocrosslinking to allow cell differentiation for tissue engineering applications such as cartilage regeneration. The functionalised polymer may be capable of forming a solid (e.g. a hydrogel) upon photocrosslinking having a storage modulus of from about 3 kPa to about 75 kPa, for example about 3 kPa, 4 kPa, 5 kPa, 6 kPa, 7 kPa, 8 kPa, 9 kPa, 10 kPa, 11 kPa, 12 kPa, 13 kPa, 14 kPa, 15 kPa, 16 kPa, 17 kPa, 18 kPa, 19 kPa, 20 kPa, 21 kPa, 22 kPa, 23 kPa, 24 kPa, 25 kPa, 26 kPa, 27 kPa, 28 kPa, 29 kPa, 30 kPa, 31 kPa, 32 kPa, 33 kPa, 34 kPa, 35 kPa, 36 kPa, 37 kPa, 38 kPa, 39 kPa, 40 kPa, 41 kPa, 42 kPa, 43 kPa, 44 kPa, 45 kPa, 46 kPa, 47 kPa, 48 kPa, 49 kPa, 50 kPa, 51 kPa, 52 kPa, 53 kPa, 54 kPa, 55 kPa, 56 kPa, 57 kPa, 58 kPa, 59 kPa, 60 kPa, 61 kPa, 62 kPa, 63 kPa, 64 kPa, 65 kPa, 66 kPa, 67 kPa, 68 kPa, 69 kPa, 70 kPa, 71 kPa, 72 kPa, 73 kPa, 74 kPa, or 75 kPa. Any minimum and maximum can be combined to form a range provided that the range is between 3 kPa to 75 kPa, such as a storage modulus of from about 5 kPa to about 50 kPa. In some embodiments, the functionalised polymer is capable of forming a solid (e.g. a hydrogel) upon photocrosslinking having a storage modulus of at from about 5 kPa to about 50 kPa, preferably from about 7 to about 37 kPa.

The polymer may be any suitable natural or synthetic polymer. The polymer may have an average molecular weight (weight average molecular weight) of from about 5 kDa to about 1000 kDa, preferably from about 68 kDa to about 780 kDa. It will be appreciated that a polymer, including synthetic and natural polymers, may be functionalised or partially functionalised to introduce desired functional groups (e.g. one or more of functional groups capable of ionic crosslinking, functional groups capable of reacting with a reagent for providing a photocrosslinkable moiety, and functional groups capable of reacting with a peptide for cell adhesion) on the polymer backbone as is known in the art. Suitable natural polymers include polysaccharides such as alginate, ulvan, hyaluronic acid (HA), chitosan, dextran sulfate, gellan gum, xanthan gum, chondroitin sulphate, agarose, cellulose and oxidised cellulose (e.g., TEMPO (2,2,6,6-tetramethylpiperidine-1-oxyl)-oxidised cellulose), and proteins such as gelatin and collagen. Suitable synthetic polymers include poly(ethylene glycol) (PEG), preferably PEG functionalised with a functional group capable of ionic crosslinking (e.g., bisphosphonate groups). In some embodiments, the polymer comprises or consists of a natural polymer, preferably a polysaccharide such as alginate, ulvan and hyaluronic acid (HA). In some embodiments, the polymer comprises or consists of one or more of alginate, ulvan, hyaluronic acid (HA), chitosan, dextran sulfate, gellan gum, xanthan gum, chondroitin sulphate, agarose, cellulose and oxidised cellulose (e.g., TEMPO (2,2,6,6-tetramethylpiperidine-1-oxyl)-oxidised cellulose), gelatin and collagen, preferably alginate. In this context, ‘consists of’ will be understood to mean that the polymer includes only the specified polymer(s) and no other polymers. In preferred embodiments, the polymer comprises or is alginate.

In preferred embodiments, the polymer comprises or consists of alginate. The alginate may be from any suitable alginate source. Alginate is composed of repeating monomeric units of α-L-guluronic acid (G) blocks and 1,4-linked β-D-mannuronic acid (M) epimers, each bearing free functional hydroxyl and carboxyl groups. The relative amount of these monomers (M/G ratio) and their arrangement, either as homopolymeric (GG or MM) or heteropolymeric (GM) blocks, as well as the molecular weight of polymer chains, can depend on the alginate source. In some embodiments, the alginate has an average molecular weight of from about 68 kDa to about 780 kDa. In some embodiments, the alginate has a M/G ratio (ratio of mannuronic acid blocks and to guluronic acid blocks) of from about 0.30 to about 2.60, for example an M/G ratio of about 0.30, 0.32, 0.34, 0.36, 0.38, 0.40, 0.42, 0.40, 0.46, 0.48, 0.50, 0.52, 0.54, 0.56, 0.58, 0.60, 0.62, 0.64, 0.66, 0.68, 0.70, 0.72, 0.74, 0.76, 0.78, 0.80, 0.82, 0.84, 0.86, 0.88, 0.90, 0.92, 0.94, 0.96, 0.98, 1.00, 1.02, 1.04, 1.06, 1.08, 1.10, 1.12, 1.14, 1.16, 1.18, 1.20, 1.22, 1.24, 1.26, 1.28, 1.30, 1.32, 1.34, 1.36, 1.38, 1.40, 1.42, 1.44, 1.46, 1.48, 1.50, 1.52, 1.54, 1.56, 1.58, 1.60, 1.62, 1.64, 1.66, 1.68, 1.70, 1.72, 1.74, 1.76, 1.78, 1.80, 1.82, 1.84, 1.86, 1.88, 1.90, 1.92, 1.94, 1.96, 1.98, 2.00, 2.02, 2.04, 2.06, 2.08, 2.10, 2.12, 2.14, 2.16, 2.18, 2.20, 2.22, 2.24, 2.26, 2.28, 2.30, 2.32, 2.34, 2.36, 2.38, 2.40, 2.42, 2.44, 2.46, 2.48, 2.50, 2.52, 2.54, 2.56, 2.58, or 2.60. Any minimum and maximum may be combined to form a range provided that the range is within 0.30 to 2.60, for example an M/G ratio of from about 0.64 to about 1.30. In some embodiments, the alginate has an M/G ratio of from about 0.64 to about 1.30.

The plurality of photocrosslinkable moieties may comprise one or more types of photocrosslinkable moiety, each independently comprising a reactive functionality capable of photocrosslinking. Each type of photocrosslinkable moiety, in particular the reactive functionality of each type of photocrosslinkable moiety, may be independently capable of crosslinking when combined with a photoinitiator and exposed to visible light. Suitable reactive functionalities include acrolyl, methacrolyl, acrylate, methacrylate, acrylamide, methacrylamide, allyl ether, maleimide, vinyl sulfone, NHS ester and vinyl ether. The person skilled in the art will be able to determine suitable photoinitiators and wavelengths of visible light suitable to allow photocrosslinking of a given reactive functionality. Each type of photocrosslinkable moiety may be independently composed of a reactive functionality linked to the polymer by a connecting group. By way of example, in embodiments where the reactive functionality is methacrylate, a photocrosslinkable moiety may be glycidyl methacrylate or aminoethyl methacrylate. Additionally, or alternatively, each type of photocrosslinkable moiety may be Independently composed of the reactive functionality. Accordingly, in some embodiments, the photocrosslinkable moiety and the reactive functionality are the same (i.e., photocrosslinkable moiety may be made up entirely of the reactive functionality).

In some embodiments, the plurality of photocrosslinkable moieties comprise or consist of methacrylate groups. In this context, ‘consist of’ will be understood to mean that the plurality of photocrosslinkable moieties contain only methacrylate groups and no other types of photocrosslinkable moieties. In preferred embodiments, the plurality of photocrosslinkable moieties is a plurality of methacrylate groups. In preferred embodiments, the plurality of photocrosslinkable moieties each comprise a methacrylate group as the reactive functionality capable of photocrosslinking.

The plurality of photocrosslinkable moieties are directly linked to the polymer. Each type of photocrosslinkable moiety may be linked to the polymer by any suitable bond or group. Suitable groups include amide and ester groups. In preferred embodiments, the plurality of photocrosslinkable moieties are each conjugated to the polymer via the oxygen atom of an ester group.

In some embodiments, the plurality of photocrosslinkable moieties each have the following structure:

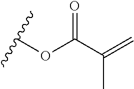 [SMILES: *OC(=O)C(=C)C]

where the wavy line denotes the point of attachment of the photocrosslinkable moiety to the polymer.

The plurality of photocrosslinkable moieties may be derived from a reagent for providing the photocrosslinkable moiety that has reacted with a functional group capable of being functionalised by the reagent present on the polymer before functionalisation. This is schematically represented in Scheme 1, where the wavy line represents the polymer backbone, FG represents the functional group capable of being functionalised, PCR represents the reagent for providing the photocrosslinkable moiety, and PCM represents the photocrosslinkable moiety.

Scheme 1, Reaction of a reagent for providing a photocrosslinkable moiety with a functional group capable of being functionalised by the reagent

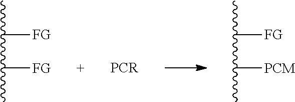

Suitable reagents and functional groups include those described herein. In some embodiments, the plurality of photocrosslinkable moieties are derived from a reagent for providing the photocrosslinkable moiety that has reacted with a hydroxyl group of the polymer. The reagent may be any reagent known in the art suitable for providing the photocrosslinkable moiety. The reagent may be selected depending on the photocrosslinkable moiety to be introduced and the functional group of the polymer to be functionalised. For example, in embodiments where the photocrosslinkable moiety comprises or is methacrylate and the functional group to be functionalised is a hydroxyl group, the reagent may be methacrylic anhydride.

The plurality of cell adhesion moieties may comprise one or more types of cell adhesion moiety each linked to the polymer via a linker. Each cell adhesion moiety may independently comprise a cell adhesion motif. As used herein, the term ‘cell adhesion motif’ will be understood to refer to an amino acid sequence that can mediate cell adhesion. Cell adhesion motifs typically bind to an extracellular matrix adhesion receptor, such as an integrin receptor or a laminin receptor. The cell adhesion motif may be any suitable motif that can be recognised by a cell and mediate cell attachment. Suitable cell adhesion motifs include integrin binding motifs such as RGD (SEQ ID NO: 1), and laminin binding motifs such as YIGSR (SEQ ID NO:7). In preferred embodiments, the cell adhesion motif is RGD. In preferred embodiments, the plurality of cell adhesion moieties each comprise RGD (i.e., an RGD cell adhesion motif).

Each cell adhesion moiety may be independently linked to a linker by any suitable bond. In preferred embodiments, each cell adhesion moiety is conjugated to the linker via a thioether bond.

In some embodiments, the plurality of cell adhesion moieties each have the following structure:

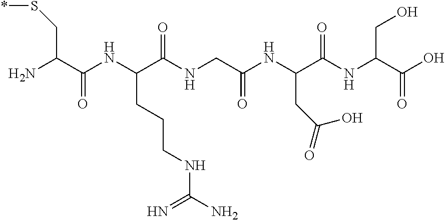 [SMILES: *SCC(N)C(=O)NC(CCCNC(=N)N)C(=O)NCC(=O)NC(CC(=O)O)C(=O)NC(CO)C(=O)O]

where the star denotes the point of attachment of the cell adhesion moiety to the linker.

Each linker may be independently derived from a photocrosslinkable moiety linked to the polymer that has reacted via its reactive functionality with a peptide for cell adhesion. It will be appreciated that the linker is not capable of photocrosslinking. Similarly, each cell adhesion moiety may be independently derived from a peptide for cell adhesion that has reacted with a reactive functionality of photocrosslinkable moiety linked to the polymer. This is depicted schematically in Scheme 2 below, where the wavy line represents the polymer backbone, PCM represents a photocrosslinkable moiety, CAP represents a peptide for cell adhesion, L represents a linker, and CAM represents a cell adhesion moiety.

Scheme 2. Reaction of a peptide for cell adhesion with a photocrosslinkable moiety

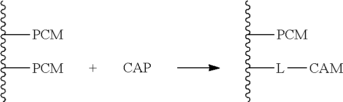

A reactive functionality of a photocrosslinkable moiety and a peptide for cell adhesion may undergo any suitable reaction known in the art. In preferred embodiments, the reaction is a thiol-Michael addition reaction. The reactive functionality and the photocrosslinkable moiety may include any of those described herein. In preferred embodiments, the reactive functionality is a methacrylate. In preferred embodiments, the photocrosslinkable moiety is a methacrylate group.

In some embodiments, the linker has the following structure:

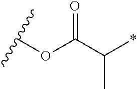 [SMILES: *OC(=O)C(*)C]

where the wavy line denotes the point of attachment of the linker to the polymer and the star denotes the point of attachment of the linker to the cell adhesion moiety.

The peptide for cell adhesion may comprise or consist of a peptide having 1 to 20, preferably 1 to 10, amino acid residues. The peptide may react via its C-terminus, N-terminus or an amino acid side chain, preferably via an amino acid side chain, more preferably via a cysteine side chain, with the reactive functionality. The non-conjugated end(s) of the peptide may have a C-terminal capping group or an N-terminal capping group.

The peptide for cell adhesion may comprise a thiol capable of undergoing a thiol-Michael addition reaction with a reactive functionality capable of photocrosslinking. In some embodiments, the peptide for cell adhesion comprises a cysteine residue. Advantageously, the cysteine sidechain may undergo a thiol-Michael reaction with a reactive functionality of a photocrosslinkable moiety (e.g., a methacrylate) to form a thioether bond.

The peptide for cell adhesion may comprise a cell adhesion motif. Suitable cell adhesion motifs include those described herein. In preferred embodiments, the peptide for cell adhesion comprises RGD.

In some embodiments, the peptide for cell adhesion comprises a cysteine residue and an RGD cell adhesion motif. In preferred embodiments, the peptide for cell adhesion is CRGDS (SEQ ID NO:6).

In some embodiments, the plurality of cell adhesion moieties linked to the polymer via a linker each have the following structure:

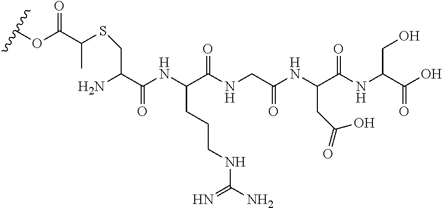 [SMILES: *OC(=O)C(C)SCC(N)C(=O)NC(CCCNC(=N)N)C(=O)NCC(=O)NC(CC(=O)O)C(=O)NC(CO)C(=O)O]

where the wavy line denotes the point of attachment of the linker to the polymer.

In some embodiments, the functionalised polymer further comprises a plurality of cell adhesion moieties directly linked to the polymer. Each cell adhesion moiety directly linked to the polymer may be independently derived from a peptide for cell adhesion that has reacted with a functional group present on the polymer before functionalisation.

In some embodiments, the functionalised polymer comprises an alginate partially functionalised with:
a plurality of methacrylate groups; and
a plurality of cell adhesion moieties comprising RGD each linked to the polymer via a linker derived from a methacrylate group that has reacted with a peptide for cell adhesion, preferably CRGDS,
   wherein preferably the functionalised polymer further comprises a plurality of carboxyl groups.

The degree of functionalisation of the polymer may be defined in one or more ways and is typically provided as a percentage. For example, the degree of polymer functionalisation may be based on the amount of functional groups capable of being functionalised (e.g. capable of being functionalised by a reagent for providing a photocrosslinkable moiety) present in the polymer that have been functionalised, relative to the polymer before functionalisation. Additionally, or alternatively, the degree of polymer functionalisation may be based on the based on the amount of monomer units making up the polymer that have been functionalised, relative to the polymer before functionalisation. It will be appreciated the functional groups capable of being functionalised may depend on the functional groups present in the polymer before functionalisation and whether the reagent for providing the photocrosslinkable moiety is capable of reacting with these groups. For example, where the polymer is alginate, the functional groups capable of being functionalised may be one or both of a hydroxyl group or a carboxyl group, depending on the reagent for providing the photocrosslinkable moiety. As another example, where the polymer is gelatin, the functional groups capable of being functionalised may be one or both of a hydroxyl group and an amine group, depending on the reagent for providing the photocrosslinkable moiety. In some embodiments, the functional groups capable of being functionalised are selected from one or more of hydroxyl groups, amine groups and carboxyl groups, preferably hydroxyl groups. The degree of functionalisation may be determined by methods known in the art including methods described herein, for example by using 1H NMR spectroscopy or elemental analysis. The person skilled in the art would understand which definitions are suitable for a given polymer and how to determine the degree of functionalisation.

In some embodiments, from about 30% to about 70% of the polymer is functionalised, for example about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 52%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69% or about 70% of the polymer is functionalised, based on the proportion of the plurality of functional groups capable of being functionalised by a reagent for providing the photocrosslinkable moiety present in the polymer before functionalisation that have been functionalised. Any minimum and maximum can be combined to form a range provided that the range is between 30% and 70%, such as a degree of functionalisation of from about 40% to about 60%. In preferred embodiments, from about 40% to about 60% of the polymer is functionalised. In embodiments where the polymer is alginate, the functional groups capable of being functionalised may be one or both of hydroxyl groups and carboxyl groups, preferably hydroxyl groups. For example, in some embodiments, from about 30% to about 70%, preferably from about 40% to about 60%, of the alginate is functionalised, based on the proportion of hydroxyl groups present in the alginate before functionalisation that have been functionalised.

In some embodiments, from 30% to 70% of the polymer is functionalised, for example 30%, 31%, 32%, 33%, 34%, 35%, 36%, 37%, 38%, 39%, 40%, 41%, 42%, 43%, 44%, 45%, 46%, 47%, 48%, 49%, 50%, 52%, 52%, 53%, 54%, 55%, 56%, 57%, 58%, 59%, 60%, 61%, 62%, 63%, 64%, 65%, 66%, 67%, 68%, 69% or 70% of the polymer is functionalised, based on the proportion of the plurality of functional groups capable of being functionalised by a reagent for providing the photocrosslinkable moiety present in the polymer before functionalisation that have been functionalised. Any minimum and maximum can be combined to form a range provided that the range is between 30% and 70%, such as a degree of functionalisation of from 40% to 60%. In preferred embodiments, from 40% to 60% of the polymer is functionalised. In embodiments where the polymer is alginate, the functional groups capable of being functionalised may be one or both of hydroxyl groups and carboxyl groups, preferably hydroxyl groups. For example, in some embodiments, from 30% to 70%, preferably from 40% to 60%, of the alginate is functionalised, based on the proportion of hydroxyl groups present in the alginate before functionalisation that have been functionalised.

In some embodiments, from about 30% to about 70% of the polymer is functionalised, for example about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 52%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69% or about 70% of the polymer is functionalised, based on the proportion of monomer units making up the polymer that have been functionalised. Any minimum and maximum can be combined to form a range provided that the range is between 30% and 70%, such as a degree of functionalisation of from about 40% to about 60%. In preferred embodiments, from about 40% to about 60% of the polymer is functionalised, based on the proportion of monomer units making up the polymer that have been functionalised. In embodiments where the polymer is alginate (which is made up of α-L-guluronic acid (G) and β-D-mannuronic acid (M) monomers units), from about 30% to about 70%, preferably from about 40% to about 60%, of the alginate may be functionalised, based on the proportion of α-L-guluronic acid (G) and β-D-mannuronic acid (M) monomers units that have been functionalised.

In some embodiments, from 30% to 70% of the polymer is functionalised, for example 30%, 31%, 32%, 33%, 34%, 35%, 36%, 37%, 38%, 39%, 40%, 41%, 42%, 43%, 44%, 45%, 46%, 47%, 48%, 49%, 50%, 52%, 52%, 53%, 54%, 55%, 56%, 57%, 58%, 59%, 60%, 61%, 62%, 63%, 64%, 65%, 66%, 67%, 68%, 69% or 70% of the polymer is functionalised, based on the proportion of monomer units making up the polymer that have been functionalised. Any minimum and maximum can be combined to form a range provided that the range is between 30% and 70%, such as a degree of functionalisation of from 40% to 60%. In preferred embodiments, from 40% to 60% of the polymer is functionalised, based on the proportion of monomer units making up the polymer that have been functionalised. In embodiments where the polymer is alginate (which is made up of α-L-guluronic acid (G) and β-D-mannuronic acid (M) monomers units), from 30% to 70%, preferably from 40% to 60%, of the alginate may be functionalised, based on the proportion of α-L-guluronic acid (G) and β-D-mannuronic acid (M) monomers units that have been functionalised.

In some embodiments, from about 16.0% to about 68.8% of the polymer is functionalised with the plurality of photocrosslinkable moieties, for example about 16.0%, about 16.1%, about 16.2%, about 16.3%, about 16.4%, about 16.5%, about 16.6%, about 16.7%, about 16.8%, about 16.9%, about 17.0%, about 17.1%, about 17.2%, about 17.3%, about 17.4%, about 17.5%, about 17.6%, about 17.7%, about 17.8%, about 17.9%, about 18.0%, about 18.1%, about 18.2%, about 18.3%, about 18.4%, about 18.5%, about 18.6%, about 18.7%, about 18.8%, about 18.9%, about 19.0%, about 19.1%, about 19.2%, about 19.3%, about 19.4%, about 19.5%, about 19.6%, about 19.7%, about 19.8%, about 19.9%, about 20.0%, about 20.1%, about 20.2%, about 20.3%, about 20.4%, about 20.5%, about 20.6%, about 20.7%, about 20.8%, about 20.9%, about 21.0%, about 21.1%, about 21.2%, about 21.3%, about 21.4%, about 21.5%, about 21.6%, about 21.7%, about 21.8%, about 21.9%, about 22.0%, about 22.1%, about 22.2%, about 22.3%, about 22.4%, about 22.5%, about 22.6%, about 22.7%, about 22.8%, about 22.9%, about 23.0%, about 23.1%, about 23.2%, about 23.3%, about 23.4%, about 23.5%, about 23.5%, about 23.6%, about 23.7%, about 23.8%, about 23.9%, about 24.0%, about 24.1%, about 24.2%, about 24.3%, about 24.4%, about 24.5%, about 24.6%, about 24.7%, about 24.8%, about 24.9%, about 25.0%, about 25.1%, about 25.2%, about 25.3%, about 25.4%, about 25.5%, about 25.6%, about 25.7%, about 25.8%, about 25.9%, about 26.0%, about 26.1%, about 26.2%, about 26.3%, about 26.4%, about 26.5%, about 26.6%, about 26.7%, about 26.8%, about 26.9%, about 27.0%, about 27.1%, about 27.2%, about 27.3%, about 27.4%, about 27.5%, about 27.6%, about 27.7%, about 27.8%, about 27.9%, about 28.0%, about 28.1%, about 28.2%, about 28.3%, about 28.4%, about 28.5%, about 28.6%, about 28.7%, about 28.8%, about 28.9%, about 29.0%, about 29.1%, about 29.2%, about 29.3%, about 29.4%, about 29.5%, about 29.6%, about 29.7%, about 29.8%, about 29.9%, about 30.0%, about 30.1%, about 30.2%, about 30.3%, about 30.1%, about 30.5%, about 30.6%, about 30.7%, about 30.8%, about 30.9%, about 31.0%, about 31.1%, about 31.2%, about 31.3%, about 31.4%, about 31.5%, about 31.6%, about 31.7%, about 31.8%, about 31.9%, about 32.0%, about 32.1%, about 32.2%, about 32.3%, about 32.4%, about 32.5%, about 32.6%, about 32.7%, about 32.8%, about 32.9%, about 33.0%, about 33.1%, about 33.2%, about 33.3%, about 33.4%, about 33.5%, about 33.6%, about 33.7%, about 33.8%, about 33.9%, about 34.0%, about 34.1%, about 34.2%, about 34.3%, about 34.4%, about 34.5%, about 34.6%, about 34.7%, about 34.8%, about 34.9%, about 35.0%, about 35.1%, about 35.2%, about 35.3%, about 35.4%, about 35.5%, about 35.6%, about 35.7%, about 35.8%, about 35.9%, about 36.0%, about 36.1%, about 36.2%, about 36.3%, about 36.4%, about 36.5%, about 36.6%, about 36.7%, about 36.8%, about 36.9%, about 37.0%, about 37.1%, about 37.2%, about 37.3%, about 37.4%, about 37.5%, about 37.6%, about 37.7%, about 37.8%, about 37.9%, about 38.0%, about 38.1%, about 38.2%, about 38.3%, about 38.4%, about 38.5%, about 38.6%, about 38.7%, about 38.8%, about 38.9%, about 39.0%, about 39.1%, about 39.2%, about 39.3%, about 39.4%, about 39.5%, about 39.6%, about 39.7%, about 39.8%, about 39.9%, about 40.0%, about 40.1%, about 40.2%, about 40.3%, about 40.4%, about 40.5%, about 40.6%, about 40.7%, about 40.8%, about 40.9%, about 41.0%, about 41.1%, about 41.2%, about 41.3%, about 41.4%, about 41.5%, about 41.6%, about 41.7%, about 41.8%, about 41.9%, about 42.0%, about 42.1%, about 42.2%, about 42.3%, about 42.4%, about 42.5%, about 42.6%, about 42.7%, about 42.8%, about 42.9%, about 43.0%, about 43.1%, about 43.2%, about 43.3%, about 43.4%, about 43.5%, about 43.6%, about 43.7%, about 43.8%, about 43.9%, about 44.0%, about 44.1%, about 44.2%, about 44.3%, about 44.4%, about 44.5%, about 44.6%, about 44.7%, about 44.8%, about 44.9%, about 45.0%, about 45.1%, about 45.2%, about 45.2%, about 45.3%, about 45.4%, about 45.5%, about 45.6%, about 45.7%, about 45.8%, about 45.9%, about 46.0%, about 46.1%, about 46.2%, about 46.3%, about 46.4%, about 46.5%, about 46.6%, about 46.7%, about 46.8%, about 46.9%, about 47.0%, about 47.1%, about 47.2%, about 47.3%, about 47.4%, about 47.5%, about 47.6%, about 47.7%, about 47.8%, about 47.9%, about 48.0%, about 48.1%, about 48.2%, about 48.3%, about 48.4%, about 48.5%, about 48.6%, about 48.7%, about 48.8%, about 48.9%, about 49.0%, about 49.1%, about 49.2%, about 49.3%, about 49.4%, about 49.5%, about 49.6%, about 49.7%, about 49.8%, about 49.9%, about 50.0%, about 50.1%, about 50.2%, about 50.3%, about 50.4%, about 50.5%, about 50.6%, about 50.7%, about 50.8%, about 50.9%, about 51.0%, about 51.5%, about 51.2%, about 51.3%, about 51.4%, about 51.5%, about 51.6%, about 51.7%, about 51.8%, about 51.9%, about 52.0%, about 52.1%, about 52.2%, about 52.3%, about 52.4%, about 52.5%, about 52.6%, about 52.7%, about 52.8%, about 52.9%, about 53.0%, about 53.1%, about 53.2%, about 53.3%, about 53.4%, about 53.5%, about 53.6%, about 53.7%, about 53.8%, about 53.9%, about 54.0%, about 54.1%, about 54.2%, about 54.3%, about 54.4%, about 54.5%, about 54.6%, about 54.7%, about 54.8%, about 54.9%, about 55.0%, about 55.1%, about 55.2%, about 55.3%, about 55.4%, about 55.5%, about 55.6%, about 55.7%, about 55.8%, about 55.9%, about 56.0%, about 56.1%, about 56.2%, about 56.3%, about 56.4%, about 56.5%, about 56.6%, about 56.7%, about 56.8%, about 56.9%, about 57.0%, about 57.1%, about 57.2%, about 57.3%, about 57.4%, about 57.5%, about 57.6%, about 57.7%, about 57.8%, about 57.9%, about 58.0%, about 58.1%, about 58.2%, about 58.3%, about 58.4%, about 58.5%, about 58.6%, about 58.7%, about 58.8%, about 58.9%, about 59.0%, about 59.1%, about 59.2%, about 59.3%, about 59.4%, about 59.5%, about 59.6%, about 59.7%, about 59.8%, about 59.9%, about 60.0%, about 60.1%, about 60.2%, about 60.3%, about 60.4%, about 60.5%, about 60.6%, about 60.7%, about 60.8%, about 60.9%, about 61.0%, about 61.1%, about 61.2%, about 61.3%, about 61.4%, about 61.5%, about 61.6%, about 61.7%, about 61.8%, about 61.9%, about 62.0%, about 62.1%, about 62.2%, about 62.3%, about 62.4%, about 62.5%, about 62.6%, about 62.7%, about 62.8%, about 62.9%, about 63.0%, about 63.1%, about 63.2%, about 63.3%, about 63.4%, about 63.5%, about 63.6%, about 63.7%, about 63.8%, about 63.9%, about 64.0%, about 64.1%, about 64.2%, about 64.3%, about 64.4%, about 64.5%, about 64.6%, about 64.7%, about 64.8%, about 64.9%, about 65.0%, about 65.1%, about 65.2%, about 65.3%, about 65.4%, about 65.5%, about 65.6%, about 65.7%, about 65.8%, about 65.9%, about 66.0%, about 66.1%, about 66.2%, about 66.3%, about 66.4%, about 66.5%, about 66.6%, about 66.7%, about 66.8%, about 66.9%, about 67.0%, about 67.1%, about 67.2%, about 67.3%, about 67.4%, about 67.5%, about 67.6%, about 67.7%, about 67.8%, about 67.9%, about 68.0%, about 68.1%, about 68.2%, about 68.3%, about 68.4%, about 68.5%, about 68.6%, about 68.7%, or about 68.8% of the polymer is functionalised with the plurality of photocrosslinkable moieties. Any minimum and maximum can be combined to form a range provided that the range is between 16.0% and 68.8%, such as a degree of functionalisation of from about 31.0% to about 58.0%. In embodiments where the plurality of photocrosslinkable moieties is a plurality of methacrylate groups, from about 16.0% to about 68.8%, preferably from about 31.0% to about 58.0%, of the polymer (e.g. alginate) may be functionalised with methacrylate. The degree of functionalisation with the photocrosslinkable moiety may be based on the proportion of the plurality of functional groups capable of being functionalised (e.g. by a reagent for providing the photocrosslinkable moiety) present in the polymer before functionalisation that have been functionalised or based on the proportion of monomer units making up the polymer that have been functionalised, as described herein.

In some embodiments, from 16.0% to 68.8% of the polymer is functionalised with the plurality of photocrosslinkable moieties, for example 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.5%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%, 19.1%, 19.2%, 19.3%, 19.4%, 19.5%, 19.6%, 19.7%, 19.8%, 19.9%, 20.0%, 20.1%, 20.2%, 20.3%, 20.4%, 20.5%, 20.6%, 20.7%, 20.8%, 20.9%, 21.0%, 21.1%, 21.2%, 21.3%, 21.4%, 21.5%, 21.6%, 21.7%, 21.8%, 21.9%, 22.0%, 22.1%, 22.2%, 22.3%, 22.4%, 22.5%, 22.6%, 22.7%, 22.8%, 22.9%, 23.0%, 23.1%, 23.2%, 23.3%, 23.4%, 23.5%, 23.5%, 23.6%, 23.7%, 23.8%, 23.9%, 24.0%, 24.1%, 24.2%, 24.3%, 24.4%, 24.5%, 24.6%, 24.7%, 24.8%, 24.9%, 25.0%, 25.1%, 25.2%, 25.3%, 25.4%, 25.5%, 25.6%, 25.7%, 25.8%, 25.9%, 26.0%, 26.1%, 26.2%, 26.3%, 26.4%, 26.5%, 26.6%, 26.7%, 26.8%, 26.9%, 27.0%, 27.1%, 27.2%, 27.3%, 27.4%, 27.5%, 27.6%, 27.7%, 27.8%, 27.9%, 28.0%, 28.1%, 28.2%, 28.3%, 28.4%, 28.5%, 28.6%, 28.7%, 28.8%, 28.9%, 29.0%, 29.1%, 29.2%, 29.3%, 29.4%, 29.5%, 29.6%, 29.7%, 29.8%, 29.9%, 30.0%, 30.1%, 30.2%, 30.3%, 30.1%, 30.5%, 30.6%, 30.7%, 30.8%, 30.9%, 31.0%, 31.1%, 31.2%, 31.3%, 31.4%, 31.5%, 31.6%, 31.7%, 31.8%, 31.9%, 32.0%, 32.1%, 32.2%, 32.3%, 32.4%, 32.5%, 32.6%, 32.7%, 32.8%, 32.9%, 33.0%, 33.1%, 33.2%, 33.3%, 33.4%, 33.5%, 33.6%, 33.7%, 33.8%, 33.9%, 34.0%, 34.1%, 34.2%, 34.3%, 34.4%, 34.5%, 34.6%, 34.7%, 34.8%, 34.9%, 35.0%, 35.1%, 35.2%, 35.3%, 35.4%, 35.5%, 35.6%, 35.7%, 35.8%, 35.9%, 36.0%, 36.1%, 36.2%, 36.3%, 36.4%, 36.5%, 36.6%, 36.7%, 36.8%, 36.9%, 37.0%, 37.1%, 37.2%, 37.3%, 37.4%, 37.5%, 37.6%, 37.7%, 37.8%, 37.9%, 38.0%, 38.1%, 38.2%, 38.3%, 38.4%, 38.5%, 38.6%, 38.7%, 38.8%, 38.9%, 39.0%, 39.1%, 39.2%, 39.3%, 39.4%, 39.5%, 39.6%, 39.7%, 39.8%, 39.9%, 40.0%, 40.1%, 40.2%, 40.3%, 40.4%, 40.5%, 40.6%, 40.7%, 40.8%, 40.9%, 41.0%, 41.1%, 41.2%, 41.3%, 41.4%, 41.5%, 41.6%, 41.7%, 41.8%, 41.9%, 42.0%, 42.1%, 42.2%, 42.3%, 42.4%, 42.5%, 42.6%, 42.7%, 42.8%, 42.9%, 43.0%, 43.1%, 43.2%, 43.3%, 43.4%, 43.5%, 43.6%, 43.7%, 43.8%, 43.9%, 44.0%, 44.1%, 44.2%, 44.3%, 44.4%, 44.5%, 44.6%, 44.7%, 44.8%, 44.9%, 45.0%, 45.1%, 45.2%, 45.2%, 45.3%, 45.4%, 45.5%, 45.6%, 45.7%, 45.8%, 45.9%, 46.0%, 46.1%, 46.2%, 46.3%, 46.4%, 46.5%, 46.6%, 46.7%, 46.8%, 46.9%, 47.0%, 47.1%, 47.2%, 47.3%, 47.4%, 47.5%, 47.6%, 47.7%, 47.8%, 47.9%, 48.0%, 48.1%, 48.2%, 48.3%, 48.4%, 48.5%, 48.6%, 48.7%, 48.8%, 48.9%, 49.0%, 49.1%, 49.2%, 49.3%, 49.4%, 49.5%, 49.6%, 49.7%, 49.8%, 49.9%, 50.0%, 50.1%, 50.2%, 50.3%, 50.4%, 50.5%, 50.6%, 50.7%, 50.8%, 50.9%, 51.0%, 51.5%, 51.2%, 51.3%, 51.4%, 51.5%, 51.6%, 51.7%, 51.8%, 51.9%, 52.0%, 52.1%, 52.2%, 52.3%, 52.4%, 52.5%, 52.6%, 52.7%, 52.8%, 52.9%, 53.0%, 53.1%, 53.2%, 53.3%, 53.4%, 53.5%, 53.6%, 53.7%, 53.8%, 53.9%, 54.0%, 54.1%, 54.2%, 54.3%, 54.4%, 54.5%, 54.6%, 54.7%, 54.8%, 54.9%, 55.0%, 55.1%, 55.2%, 55.3%, 55.4%, 55.5%, 55.6%, 55.7%, 55.8%, 55.9%, 56.0%, 56.1%, 56.2%, 56.3%, 56.4%, 56.5%, 56.6%, 56.7%, 56.8%, 56.9%, 57.0%, 57.1%, 57.2%, 57.3%, 57.4%, 57.5%, 57.6%, 57.7%, 57.8%, 57.9%, 58.0%, 58.1%, 58.2%, 58.3%, 58.4%, 58.5%, 58.6%, 58.7%, 58.8%, 58.9%, 59.0%, 59.1%, 59.2%, 59.3%, 59.4%, 59.5%, 59.6%, 59.7%, 59.8%, 59.9%, 60.0%, 60.1%, 60.2%, 60.3%, 60.4%, 60.5%, 60.6%, 60.7%, 60.8%, 60.9%, 61.0%, 61.1%, 61.2%, 61.3%, 61.4%, 61.5%, 61.6%, 61.7%, 61.8%, 61.9%, 62.0%, 62.1%, 62.2%, 62.3%, 62.4%, 62.5%, 62.6%, 62.7%, 62.8%, 62.9%, 63.0%, 63.1%, 63.2%, 63.3%, 63.4%, 63.5%, 63.6%, 63.7%, 63.8%, 63.9%, 64.0%, 64.1%, 64.2%, 64.3%, 64.4%, 64.5%, 64.6%, 64.7%, 64.8%, 64.9%, 65.0%, 65.1%, 65.2%, 65.3%, 65.4%, 65.5%, 65.6%, 65.7%, 65.8%, 65.9%, 66.0%, 66.1%, 66.2%, 66.3%, 66.4%, 66.5%, 66.6%, 66.7%, 66.8%, 66.9%, 67.0%, 67.1%, 67.2%, 67.3%, 67.4%, 67.5%, 67.6%, 67.7%, 67.8%, 67.9%, 68.0%, 68.1%, 68.2%, 68.3%, 68.4%, 68.5%, 68.6%, 68.7%, or 68.8% of the polymer is functionalised with the plurality of photocrosslinkable moieties. Any minimum and maximum can be combined to form a range provided that the range is between 16.0% and 68.8%, such as a degree of functionalisation of from 19.5% to 68.5% or from 31.0% to 58.0%. In embodiments where the plurality of photocrosslinkable moieties is a plurality of methacrylate groups, from 16.0% to 68.8%, preferably from 19.5% to 68.5%, more preferably from 31.0% to 58.0%, of the polymer (e.g. alginate) may be functionalised with methacrylate. The degree of functionalisation with the photocrosslinkable moiety may be based on the proportion of the plurality of functional groups capable of being functionalised (e.g. by a reagent for providing the photocrosslinkable moiety) present in the polymer before functionalisation that have been functionalised or based on the proportion of monomer units making up the polymer that have been functionalised, as described herein.

In some embodiments, from about 1.2% to about 14.0% of the polymer is functionalised with the plurality of cell adhesion moieties (each linked to the polymer via a linker), for example about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, about 10.0%, about 10.1%, about 10.2%, about 10.3%, about 10.4%, about 10.5%, about 10.6%, about 10.7%, about 10.8%, about 10.9%, about 11.0%, about 11.1%, about 11.2%, about 11.3%, about 11.4%, about 11.5%, about 11.6%, about 11.7%, about 11.8%, about 11.9%, about 12.0%, about 12.1%, about 12.2%, about 12.3%, about 12.4%, about 12.5%, about 12.6%, about 12.7%, about 12.8%, about 12.9%, about 13.0%, about 13.1%, about 13.2%, about 13.3%, about 13.4%, about 13.5%, about 13.6%, about 13.7%, about 13.8%, about 13.9%, or about 14.0% of the polymer is functionalised with the plurality of cell adhesion moieties. Any minimum and maximum can be combined to form a range provided that the range is between 1.2% and 14.0%, such as a degree of functionalisation of from about 1.5% to about 10.5% or from about 2.0% to about 9.0%. In embodiments where the plurality of cell adhesion moieties each comprise RGD, from about 1.2% to about 14.0%, preferably from about 1.5% to about 10.5%, more preferably from about 2.0% to about 9.0%, of the polymer (e.g. alginate) may be functionalised with cell adhesion moieties comprising RGD. The degree of functionalisation with the plurality of cell adhesion moieties may be based on the proportion of the plurality of functional groups capable of being functionalised (e.g. by a reagent for providing the photocrosslinkable moiety, from which the linker may be derived) present in the polymer before functionalisation that have been functionalised or based on the proportion of monomer units making up the polymer that have been functionalised, as described herein.

In some embodiments, from 1.2% to 14.0% of the polymer is functionalised with the plurality of cell adhesion moieties (each linked to the polymer via a linker), for example 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, or 14.0% of the polymer is functionalised with the plurality of cell adhesion moieties. Any minimum and maximum can be combined to form a range provided that the range is between 1.2% and 14.0%, such as a degree of functionalisation of from about 1.5% to about 10.5% or from about 2.0% to about 9.0%. In embodiments where the plurality of cell adhesion moieties each comprise RGD, from about 1.2% to about 14.0%, preferably from about 1.5% to about 10.5%, more preferably from about 2.0% to about 9.0%, of the polymer (e.g. alginate) may be functionalised with cell adhesion moieties comprising RGD. The degree of functionalisation with the plurality of cell adhesion moieties may be based on the proportion of the plurality of functional groups capable of being functionalised (e.g. by a reagent for providing the photocrosslinkable moiety, from which the linker may be derived) present in the polymer before functionalisation that have been functionalised or based on the proportion of monomer units making up the polymer that have been functionalised, as described herein.

In some embodiments, the functionalised polymer comprises an alginate partially functionalised with:
a plurality of methacrylate groups, wherein from 16.0% to 68.8%, preferably from 19.5% to 68.5%, more preferably from 31% to 58%, of the alginate is functionalised with methacrylate; and
a plurality of cell adhesion moieties comprising RGD each linked to the polymer via a linker derived from a methacrylate group that has reacted with a peptide for cell adhesion, preferably CRGDS, wherein from about 1.2% to about 14.0%, preferably from about 1.5% to about 10.5%, more preferably from about 2.0% to about 9%, of the alginate is functionalised with the cell adhesion moieties, wherein preferably the functionalised polymer further comprises a plurality of carboxyl groups.

The functionalised polymer may be derived from a polymer partially functionalised with a plurality of photocrosslinkable moieties each comprising a reactive functionality capable of photocrosslinking that has reacted with a peptide for cell adhesion, wherein the peptide for cell adhesion has reacted with the reactive functionality of a portion of the plurality of the photocrosslinkable moieties present on the polymer to thereby provide the plurality of cell adhesion moieties linked to the polymer via a linker. It will be understood that in these embodiments, the linker is derived from a photocrosslinkable moiety that has reacted via its reactive functionality with a peptide for cell adhesion, and the plurality of cell adhesion moieties are derived from the peptide for cell adhesion that has reacted with the reactive functionality of a portion of the plurality photocrosslinkable moieties. This is depicted schematically in Scheme 2 above.

The polymer partially functionalised with a plurality of photocrosslinkable moieties may be any polymer functionalised with any photocrosslinkable moiety as described herein. In some embodiments, the polymer partially functionalised with a plurality of photocrosslinkable moieties may be alginate partially functionalised with methacrylate groups.

Before reaction with the peptide for cell adhesion, from about 30% to about 70% of the polymer may have been functionalised with the plurality of photocrosslinkable moieties, for example about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 52%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, or about 70% of the polymer may have been functionalised with the plurality of photocrosslinkable moieties. Any minimum and maximum can be combined to form a range provided that the range is between 30% and 70%, such as a degree of functionalisation of from about 40% to about 60%. In embodiments where the polymer partially functionalised with a plurality of photocrosslinkable moieties is alginate partially functionalised with methacrylate groups, from about 30% to about 70%, preferably from about 40% to about 60%, of the alginate may be functionalised with methacrylate. The degree of functionalisation with the photocrosslinkable moiety may be based on the proportion of the plurality of functional groups capable of being functionalised (e.g. by a reagent for providing the photocrosslinkable moiety) present in the polymer before functionalisation that have been functionalised or based on the proportion of monomer units making up the polymer that have been functionalised, as described herein.

Before reaction with the peptide for cell adhesion, from 30% to 70% of the polymer may have been functionalised with the plurality of photocrosslinkable moieties, for example 30%, 31%, 32%, 33%, 34%, 35%, 36%, 37%, 38%, 39%, 40%, 41%, 42%, 43%, 44%, 45%, 46%, 47%, 48%, 49%, 50%, 52%, 52%, 53%, 54%, 55%, 56%, 57%, 58%, 59%, 60%, 61%, 62%, 63%, 64%, 65%, 66%, 67%, 68%, 69% or 70% of the polymer may have been functionalised with the plurality of photocrosslinkable moieties. Any minimum and maximum can be combined to form a range provided that the range is between 30% and 70%, such as a degree of functionalisation of from 40% to 60%. In embodiments where the polymer partially functionalised with a plurality of photocrosslinkable moieties is alginate partially functionalised with methacrylate groups, from 30% to 70%, preferably from 40% to 60%, of the alginate may be functionalised with methacrylate. The degree of functionalisation with the photocrosslinkable moiety may be based on the proportion of the plurality of functional groups capable of being functionalised (e.g. by a reagent for providing the photocrosslinkable moiety) present in the polymer before functionalisation that have been functionalised or based on the proportion of monomer units making up the polymer that have been functionalised, as described herein.

The peptide for cell adhesion may be any such peptide as described herein. The peptide for cell adhesion may react with the reactive functionality of from about 4% to about 20% of the plurality of the photocrosslinkable moieties present on the polymer, for example about 4%, about 5%, about 6%, about 7%, about 8%, about 9%, about 10%, about 11%, about 12%, about 13%, about 14%, about 15%, about 16%, about 17%, about 18%, about 19%, or about 20% of the plurality of photocrosslinkable moieties present on the polymer, based on the proportion of photocrosslinkable moieties present on the polymer before reaction that have reacted a the peptide for cell adhesion. Any minimum and maximum can be combined to form a range provided that the range is between 4% and 20%, such as from about 5% to about 15% of the plurality of the photocrosslinkable moieties present on the polymer.

The peptide for cell adhesion may react with the reactive functionality of from 4% to 20% of the plurality of the photocrosslinkable moieties present on the polymer, for example 4%, 5%, 6%, 7%, 8%, 9%, 10%, 11%, 12%, 13%, 14%, 15%, 16%, 17%, 18%, 19%, or 20% of the plurality of photocrosslinkable moieties present on the polymer, based on the proportion of photocrosslinkable moieties present on the polymer before reaction that have reacted a the peptide for cell adhesion. Any minimum and maximum can be combined to form a range provided that the range is between 4% and 20%, such as from 5% to 15% of the plurality of the photocrosslinkable moieties present on the polymer.

In some embodiments, the functionalised polymer is derived from alginate partially functionalised with methacrylate that has reacted with a peptide for cell adhesion comprising a cysteine, preferably CRGDS, wherein the peptide for cell adhesion has reacted with a portion of the methacrylate present on the alginate.

In some embodiments, the functionalised polymer is capable of a liquid to solid phase change caused by an ionic crosslinking agent, preferably a divalent cation, preferably Ca²⁺. The formed solid (i.e., the functionalised polymer upon ionic crosslinking) may preferably have a storage modulus of from about 3 kPa to about 72 kPa, for example about 3 kPa, 4 kPa, 5 kPa, 6 kPa, 7 kPa, 8 kPa, 9 kPa, 10 kPa, 11 kPa, 12 kPa, 13 kPa, 14 kPa, 15 kPa, 16 kPa, 17 kPa, 18 kPa, 19 kPa, 20 kPa, 21 kPa, 22 kPa, 23 kPa, 24 kPa, 25 kPa, 26 kPa, 27 kPa, 28 kPa, 29 kPa, 30 kPa, 31 kPa, 32 kPa, 33 kPa, 34 kPa, 35 kPa, 36 kPa, 37 kPa, 38 kPa, 39 kPa, 40 kPa, 41 kPa, 42 kPa, 43 kPa, 44 kPa, 45 kPa, 46 kPa, 47 kPa, 48 kPa, 49 kPa, 50 kPa, 51 kPa, 52 kPa, 53 kPa, 54 kPa, 55 kPa, 56 kPa, 57 kPa, 58 kPa, 59 kPa, 60 kPa, 61 kPa, 62 kPa, 63 kPa, 64 kPa, 65 kPa, 66 kPa, 67 kPa, 68 kPa, 69 kPa, 70 kPa, 71 kPa, or 72 kPa. Any minimum and maximum can be combined to form a range provided that the range is between 3 kPa to 72 kPa, such as a storage modulus of from about 5 kPa to about 50 kPa. In some embodiments, the functionalised polymer is of forming a solid (e.g. a hydrogel) upon ionic crosslinking having a storage modulus of at from about 5 kPa to about 50 kPa, preferably from about 10 kPa to about 50 kPa.

In some embodiments, the functionalised polymer is capable of adhering cells, preferably upon ionic crosslinking. The functionalised polymer may preferably exhibit at least about 80%, 81%, 82%, 83%, 84%, 85%, 86%, 87%, 88%, 89%, 90%, 91%, 92%, 93%, 94%, 95%, 96%, 97%, 98% or 99% cell adhesion, where percent adhesion is calculated according to the following equation:

$$\% adhesion = ( \frac{Seeded Cells - Unattached Cells}{Seeded cells} ) \times 100$$

In some embodiments, the functionalised polymer is capable of a solid to liquid phase change caused by a chelator chelating an ionic crosslinking agent, preferably wherein the chelator is ethylenediaminetetraacetic acid (EDTA). The functionalised polymer may preferably undergo a solid to liquid phase change caused by a chelator chelating an ionic crosslinking agent within about 30 minutes, for example about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 minutes. Any minimum and maximum can be combined to form a range provided that the range is within 30 minutes, such as within from about 5 minutes to about 25 minutes, preferably within from about 5 minutes to about 15 minutes. The chelator may be EDTA, preferably from about 0.5 mM to about 250 mM, more preferably 250 mM EDTA, which may be added at a volume ratio of functionalised polymer to EDTA of about 3:1.

In some embodiments, the functionalised polymer is capable of photocrosslinking, preferably by combining the functionalised polymer with a photoinitiator and exposing to visible light. The functionalised polymer upon photocrosslinking may preferably be castable. The photocrosslinked polymer may preferably have a storage modulus of at least about 3 kPa to about 75 kPa, for example about 3 kPa, 4 kPa, 5 kPa, 6 kPa, 7 kPa, 8 kPa, 9 kPa, 10 kPa, 11 kPa, 12 kPa, 13 kPa, 14 kPa, 15 kPa, 16 kPa, 17 kPa, 18 kPa, 19 kPa, 20 kPa, 21 kPa, 22 kPa, 23 kPa, 24 kPa, 25 kPa, 26 kPa, 27 kPa, 28 kPa, 29 kPa, 30 kPa, 31 kPa, 32 kPa, 33 kPa, 34 kPa, 35 kPa, 36 kPa, 37 kPa, 38 kPa, 39 kPa, 40 kPa, 41 kPa, 42 kPa, 43 kPa, 44 kPa, 45 kPa, 46 kPa, 47 kPa, 48 kPa, 49 kPa, 50 kPa, 51 kPa, 52 kPa, 53 kPa, 54 kPa, 55 kPa, 56 kPa, 57 kPa, 58 kPa, 59 kPa, 60 kPa, 61 kPa, 62 kPa, 63 kPa, 64 kPa, 65 kPa, 66 kPa, 67 kPa, 68 kPa, 69 kPa, 70 kPa, 71 kPa, 72 kPa, 73 kPa, 74 kPa, or 75 kPa. Any minimum and maximum can be combined to form a range provided that the range is between 3 kPa to 75 kPa, such as a storage modulus of from about 5 kPa to about 50 kPa. In some embodiments, the polymer composition is of forming a solid (e.g. a hydrogel) upon photocrosslinking having a storage modulus of at from about 5 kPa to about 50 kPa, preferably from about 7 to about 37 kPa.

In some embodiments, the functionalised polymer is castable, for example upon photocrosslinking. The term “castable” will be understood to refer to the ability to retrieve a certain amount of the polymer through a syringe or a pipette and to extrude the said amount in a vessel.

The polymer may be referred to as a biopolymer indicating suitability for in vivo use in a human or non-human animal.

### Methods of Preparation

The present invention also provides a method for preparing the functionalised polymer described herein, that is, a functionalised polymer which comprises a polymer partially functionalised with a plurality of photocrosslinkable moieties directly linked to the polymer, each photocrosslinkable moiety comprising a reactive functionality capable of photocrosslinking, and a plurality of cell adhesion moieties each linked to the polymer via a linker, each cell adhesion moiety comprising a cell adhesion motif. The functionalised polymer may preferably further comprise a plurality of functional groups capable of ionically crosslinking with an ionic crosslinking agent, as described herein.

In one aspect, the method comprises:
providing a polymer partially functionalised with a plurality of photocrosslinkable moieties, each photocrosslinkable moiety comprising a reactive functionality capable of photocrosslinking; and
reacting a peptide for cell adhesion with a portion of the plurality of the photocrosslinkable moieties present on the polymer, to thereby provide the plurality of cell adhesion moieties linked to the polymer via a linker;
   thereby providing the functionalised polymer.

In another aspect, the method comprises:
providing a polymer comprising a plurality of functional groups, preferably a plurality of functional groups capable of reacting with a reagent for providing a photocrosslinkable moiety;
reacting a reagent for providing a photocrosslinkable moiety with a portion of the plurality of functional groups present on the polymer, to provide a polymer partially functionalised with a plurality of photocrosslinkable moieties; and
reacting a peptide for cell adhesion with a portion of the plurality of photocrosslinkable moieties present on the polymer, to thereby provide the plurality of cell adhesion moieties linked to the polymer via a linker;
   thereby providing the functionalised polymer.

It will be understood that the linker may derived from a photocrosslinkable moiety that has reacted via its reactive functionality with a peptide for cell adhesion, and the plurality of cell adhesion moieties are derived from the peptide for cell adhesion that has reacted with the reactive functionality of a portion of the plurality photocrosslinkable moieties, as described herein. This is depicted schematically in Scheme 2 above.

The features of the components used in the methods, for example the polymers, functional groups of the polymer capable of being functionalised, reagents for providing photocrosslinkable moieties, photocrosslinkable moieties, reactive functionalities capable of photocrosslinking, peptides for cell adhesion, cell adhesion moieties and linkers, include those described herein.

In some embodiments, the step of reacting a peptide for cell adhesion comprises reacting the peptide for cell adhesion with a portion of the plurality of photocrosslinkable moieties present on the polymer via a thiol-Michael addition reaction. In these embodiments, the peptide for cell adhesion may preferably comprise a cysteine residue.

In some embodiments, the preparation method comprises:
providing alginate partially functionalised with methacrylate groups; and
reacting a peptide for cell adhesion, preferably CRGDS, with a portion of the methacrylate groups;
   thereby providing alginate partially functionalised with a plurality of methacrylate groups and a plurality of cell adhesion moieties comprising RGD each linked to the polymer via a linker derived from a methacrylate group that has reacted with the peptide for cell adhesion, preferably CRGDS (SEQ ID NO:6).

In some embodiments, the preparation method comprises:
providing alginate;
reacting methacrylic anhydride with a portion of the hydroxyl groups of alginate to provide alginate partially functionalised with methacrylate groups; and
reacting a peptide for cell adhesion, preferably CRGDS, with a portion of the methacrylate groups;
   thereby providing alginate partially functionalised with a plurality of methacrylate groups and a plurality of cell adhesion moieties comprising RGD each linked to the polymer via a linker derived from a methacrylate group that has reacted with the peptide for cell adhesion, preferably CRGDS (SEQ ID NO: 6).

In some embodiments, the polymer partially functionalised with a plurality of photocrosslinkable moieties is from about 30% to about 70% functionalised with the plurality of photocrosslinkable moieties, for example about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 52%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69% or about 70% functionalised with the plurality of photocrosslinkable moieties. Any minimum and maximum can be combined to form a range provided that the range is between 30% and 70%, such as from about 40% to about 60% functionalised with the plurality of photocrosslinkable moieties. In embodiments where the polymer partially functionalised with a plurality of photocrosslinkable moieties is alginate partially functionalised with methacrylate groups, the alginate may be from about 30% to about 70%, preferably from about 40% to about 60%, functionalised with the methacrylate groups.

In some embodiments, the polymer partially functionalised with a plurality of photocrosslinkable moieties is from 30% to 70% functionalised with the plurality of photocrosslinkable moieties, for example 30%, 31%, 32%, 33%, 34%, 35%, 36%, 37%, 38%, 39%, 40%, 41%, 42%, 43%, 44%, 45%, 46%, 47%, 48%, 49%, 50%, 52%, 52%, 53%, 54%, 55%, 56%, 57%, 58%, 59%, 60%, 61%, 62%, 63%, 64%, 65%, 66%, 67%, 68%, 69% or 70% functionalised with the plurality of photocrosslinkable moieties. Any minimum and maximum can be combined to form a range provided that the range is between 30% and 70%, such as from 40% to 60% functionalised with the plurality of photocrosslinkable moieties. In embodiments where the polymer partially functionalised with a plurality of photocrosslinkable moieties is alginate partially functionalised with methacrylate groups, the alginate may be from 30% to 70%, preferably from 40% to 60%, functionalised with the methacrylate groups.

In some embodiments, the step of reacting a reagent for providing a photocrosslinkable moiety comprises reacting the reagent with from about 30% to about 70% of the plurality of functional groups present on the polymer, for example about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 52%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69% or about 70% of the plurality of functional groups present on the polymer. Any minimum and maximum can be combined to form a range provided that the range is between 30% and 70%, such as from about 40% to about 60% of the plurality of functional groups. In embodiments where the reagent is methacrylic anhydride and the polymer is alginate, the methacrylic anhydride may react with from about 30% to about 70%, preferably from about 40% to about 60%, of the hydroxyl groups present in the alginate. It will be understood that the proportion of the plurality of functional groups that have reacted with the reagent correlates to the degree of functionalisation with the photocrosslinkable moiety in the formed polymer product. For example, reacting the reagent with from about 30% to about 70% of the plurality of functional groups present in the polymer would provide a polymer that is from about 30% to about 70% functionalised with the photocrosslinkable moiety. The person skilled in the art would be able to determine the reagents and conditions required to achieve a desired extent of reaction or functionalisation.

In some embodiments, the step of reacting a reagent for providing a photocrosslinkable moiety comprises reacting the reagent with from 30% to 70% of the plurality of functional groups present on the polymer, for example 30%, 31%, 32%, 33%, 34%, 35%, 36%, 37%, 38%, 39%, 40%, 41%, 42%, 43%, 44%, 45%, 46%, 47%, 48%, 49%, 50%, 52%, 52%, 53%, 54%, 55%, 56%, 57%, 58%, 59%, 60%, 61%, 62%, 63%, 64%, 65%, 66%, 67%, 68%, 69% or 70% of the plurality of functional groups present on the polymer. Any minimum and maximum can be combined to form a range provided that the range is between 30% and 70%, such as from 40% to 60% of the plurality of functional groups. In embodiments where the reagent is methacrylic anhydride and the polymer is alginate, the methacrylic anhydride may react with from 30% to 70%, preferably from 40% to 60%, of the hydroxyl groups present in the alginate. It will be understood that the proportion of the plurality of functional groups that have reacted with the reagent correlates to the degree of functionalisation with the photocrosslinkable moiety in the formed polymer product. For example, reacting the reagent with from 30% to 70% of the plurality of functional groups present in the polymer would provide a polymer that is from 30% to 70% functionalised with the photocrosslinkable moiety. The person skilled in the art would be able to determine the reagents and conditions required to achieve a desired extent of reaction or functionalisation.

In some embodiments, the step of reacting a peptide for cell adhesion comprises reacting the peptide for cell adhesion with from about 4% to about 20% of the photocrosslinkable moieties present on the polymer, for example about 4%, about 5%, about 6%, about 7%, about 8%, about 9%, about 10%, about 11%, about 12%, about 13%, about 14%, about 15%, about 16%, about 17%, about 18%, about 19%, or about 20% of the photocrosslinkable moieties present on the polymer, based on the proportion of photocrosslinkable moieties present on the polymer before reaction that have reacted the peptide for cell adhesion. Any minimum and maximum can be combined to form a range provided that the range is between 4% and 20%, such as from about 5% to about 15% of the photocrosslinkable moieties present on the polymer. In embodiments where the peptide for cell adhesion is CRGDS (SEQ ID NO: 6) and photocrosslinkable moieties comprise or are methacrylate groups, the CRGDS (SEQ ID NO: 6) may react with from about 4% to about 20%, preferably from about 5% to about 15%, more preferably from about 5% to about 10% of the methacrylate groups. It will be understood that the proportion of the photocrosslinkable moieties that have reacted with the peptide for cell adhesion can be used to determine the degree of functionalisation with the photocrosslinkable moiety and the cell adhesion moiety in the formed polymer product. For example, reacting the peptide for cell adhesion with from about 5% to about 15% of the photocrosslinkable moieties present on a polymer that is from about 30% to about 70% functionalised with the photocrosslinkable moieties would provide a polymer that is from about 19.5% to about 68.5% functionalised with the photocrosslinkable moiety and from about 1.5% to about 10.5% functionalised with the peptide for cell adhesion. The person skilled in the art would be able to determine the reagents and conditions required to achieve a desired extent of reaction or functionalisation.

In some embodiments, the step of reacting a peptide for cell adhesion comprises reacting the peptide for cell adhesion with from 4% to 20% of the photocrosslinkable moieties present on the polymer, for example 4%, 5%, 6%, 7%, 8%, 9%, 10%, 11%, 12%, 13%, 14%, 15%, 16%, 17%, 18%, 19%, or 20% of the photocrosslinkable moieties present on the polymer, based on the proportion of photocrosslinkable moieties present on the polymer before reaction that have reacted the peptide for cell adhesion. Any minimum and maximum can be combined to form a range provided that the range is between 4% and 20%, such as from 5% to 15% of the photocrosslinkable moieties present on the polymer. In embodiments where the peptide for cell adhesion is CRGDS (SEQ ID NO: 6) and photocrosslinkable moieties comprise or are methacrylate groups, the CRGDS (SEQ ID NO: 6) may react with from 4% to 20%, preferably from 5% to 15%, more preferably from 5% to 10% of the methacrylate groups. It will be understood that the proportion of the photocrosslinkable moieties that have reacted with the peptide for cell adhesion can be used to determine the degree of functionalisation with the photocrosslinkable moiety and the cell adhesion moiety in the formed polymer product. For example, reacting the peptide for cell adhesion with from 5% to 15% of the photocrosslinkable moieties present on a polymer that is from about 30% to about 70% functionalised with the photocrosslinkable moieties would provide a polymer that is from 19.5% to 68.5% functionalised with the photocrosslinkable moiety and from 1.5% to 10.5% functionalised with the peptide for cell adhesion. The person skilled in the art would be able to determine the reagents and conditions required to achieve a desired extent of reaction or functionalisation.

The present invention also provides the functionalised polymer prepared by a method described herein.

### Polymer Composition

The present invention also provides a polymer composition comprising the functionalised polymer described herein or prepared by a method described herein, and an aqueous solution. As described herein and demonstrated in the Examples, the functionalised polymer may advantageously be useful for forming a hydrogel.

The aqueous solution may be any aqueous solution suitable for forming a hydrogel. Suitable aqueous solutions include water and buffer (or salt) solutions such as phosphate buffered saline (PBS), triethanolamine (TEOA) buffered saline, 4-(2-hydroxyethyl)-1-piperazineethanesulfonic acid) (HEPES) buffered saline or a cell culture medium such as Dulbecco's Modified Eagle Medium (DMEM). The aqueous solution may preferably be isotonic relative to a cell to be delivered by the polymer composition. In some embodiments, the aqueous solution is a buffer solution, preferably selected from PBS, TEOA buffered saline, HEPES buffered saline and DMEM, more preferably selected from HEPES buffered saline and DMEM.

The functionalised polymer may be provided in any amount suitable for forming a hydrogel. In some embodiments, the polymer composition comprises the polymer in an amount of from about 3% w/v to about 10% w/v, preferably from about 5% w/v to about 8% w/v, based on the volume of aqueous solution. In some embodiments, the polymer composition comprises the polymer in an amount of about 3% w/v, 4% w/v, 5% w/v, 6% w/v, 7% w/V, 8% w/v, 9% w/v, or 10% w/v. It will be understood that the weight of functionalised polymer present in the polymer composition may differ to the final solid content present in a hydrogel prepared from that composition.

The polymer composition may comprise one or more types of functionalised suitable for forming a hydrogel, including those described herein. In some embodiments, the polymer composition comprises predominantly one type of functionalised polymer, for example from about 50%, 55%, 60%, 65%, 70%, 75%, 80% 85%, 90% or 95% of one type of functionalised polymer. In preferred embodiments, the polymer comprises one type of functionalised polymer.

Preferably, the polymer composition is capable of forming a hydrogel having the following properties: 1. Cellular adhesion, 2. Inducible phase change, preferably reversible phase change, 3. Crosslinkability. In this context, ‘phase change’ will be understood to refer to a change in physical state, for example a change to a solid (including gel) or a liquid phase.

In some embodiments, the polymer composition is capable of a liquid to solid phase change caused by an ionic crosslinking agent, preferably a divalent cation, preferably Ca²⁺. The polymer composition may preferably be capable of forming a solid (e.g. a hydrogel) upon ionic crosslinking having a storage modulus which forms a stable substrate for cell culture for at least 1 day, 2 days, 3 days, 4 days, 5 days, 6 days, or 7 days, preferably at least 7 days. The formed solid (i.e., the polymer composition upon ionic crosslinking) may preferably have a storage modulus of from about 3 kPa to about 72 kPa, for example about 3 kPa, 4 kPa, 5 kPa, 6 kPa, 7 kPa, 8 kPa, 9 kPa, 10 kPa, 11 kPa, 12 kPa, 13 kPa, 14 kPa, 15 kPa, 16 kPa, 17 kPa, 18 kPa, 19 kPa, 20 kPa, 21 kPa, 22 kPa, 23 kPa, 24 kPa, 25 kPa, 26 kPa, 27 kPa, 28 kPa, 29 kPa, 30 kPa, 31 kPa, 32 kPa, 33 kPa, 34 kPa, 35 kPa, 36 kPa, 37 kPa, 38 kPa, 39 kPa, 40 kPa, 41 kPa, 42 kPa, 43 kPa, 44 kPa, 45 kPa, 46 kPa, 47 kPa, 48 kPa, 49 kPa, 50 kPa, 51 kPa, 52 kPa, 53 kPa, 54 kPa, 55 kPa, 56 kPa, 57 kPa, 58 kPa, 59 kPa, 60 kPa, 61 kPa, 62 kPa, 63 kPa, 64 kPa, 65 kPa, 66 kPa, 67 kPa, 68 kPa, 69 kPa, 70 kPa, 71 kPa, or 72 kPa. Any minimum and maximum can be combined to form a range provided that the range is between 3 kPa to 72 kPa, such as a storage modulus of from about 5 kPa to about 50 kPa. In some embodiments, the polymer composition is of forming a solid (e.g. a hydrogel) upon ionic crosslinking having a storage modulus of at from about 5 kPa to about 50 kPa, preferably from about 10 kPa to about 50 kPa.

In some embodiments, the polymer composition is capable of adhering cells, preferably upon ionic crosslinking. The polymer composition may preferably exhibit at least about 80%, 81%, 82%, 83%, 84%, 85%, 86%, 87%, 88%, 89%, 90%, 91%, 92%, 93%, 94%, 95%, 96%, 97%, 98% or 99% cell adhesion, where percent adhesion is calculated according to the following equation:

$$\% adhesion = ( \frac{Seeded Cells - Unattached Cells}{Seeded cells} ) \times 100$$

In some embodiments, the polymer composition is capable of a solid to liquid phase change caused by a chelator chelating an ionic crosslinking agent, preferably wherein the chelator is ethylenediaminetetraacetic acid (EDTA). The polymer composition may preferably undergo a solid to liquid phase change caused by chelator chelating an ionic crosslinking agent within about 30 minutes, for example about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 minutes. Any minimum and maximum can be combined to form a range provided that the range is within 30 minutes, such as within from about 5 minutes to about 25 minutes, preferably within from about 5 minutes to about 15 minutes. The chelator may be EDTA, preferably from about 0.5 mM to about 250 mM, more preferably 250 mM EDTA, which may be added at a volume ratio of functionalised polymer to EDTA of about 3:1.

In some embodiments, the polymer composition is capable of photocrosslinking, preferably by combining the polymer composition with a photoinitiator and exposing to visible light. The polymer composition upon photocrosslinking may preferably be castable. The photocrosslinked polymer composition may preferably have a storage modulus of at least about 3 kPa to about 75 kPa, for example about 3 kPa, 4 kPa, 5 kPa, 6 kPa, 7 kPa, 8 kPa, 9 kPa, 10 kPa, 11 kPa, 12 kPa, 13 kPa, 14 kPa, 15 kPa, 16 kPa, 17 kPa, 18 kPa, 19 kPa, 20 kPa, 21 kPa, 22 kPa, 23 kPa, 24 kPa, 25 kPa, 26 kPa, 27 kPa, 28 kPa, 29 kPa, 30 kPa, 31 kPa, 32 kPa, 33 kPa, 34 kPa, 35 kPa, 36 kPa, 37 kPa, 38 kPa, 39 kPa, 40 kPa, 41 kPa, 42 kPa, 43 kPa, 44 kPa, 45 kPa, 46 kPa, 47 kPa, 48 kPa, 49 kPa, 50 kPa, 51 kPa, 52 kPa, 53 kPa, 54 kPa, 55 kPa, 56 kPa, 57 kPa, 58 kPa, 59 kPa, 60 kPa, 61 kPa, 62 kPa, 63 kPa, 64 kPa, 65 kPa, 66 kPa, 67 kPa, 68 kPa, 69 kPa, 70 kPa, 71 kPa, 72 kPa, 73 kPa, 74 kPa, or 75 kPa. Any minimum and maximum can be combined to form a range provided that the range is between 3 kPa to 75 kPa, such as a storage modulus of from about 5 kPa to about 50 kPa. In some embodiments, the polymer composition is capable of forming a solid (e.g. a hydrogel) upon photocrosslinking having a storage modulus of at from about 5 kPa to about 50 kPa, preferably from about 7 to about 37 kPa.

In some embodiments, the polymer composition is castable, for example upon photocrosslinking. The term “castable” will be understood to refer to the ability to retrieve a certain amount of the polymer composition through a syringe or a pipette and to extrude the said amount in a vessel.

### Application

As described herein and demonstrated in the Examples, the functionalised polymer described herein may be useful in methods, uses and compositions for providing cells for implementation in cell therapy and/or surgical techniques.

Accordingly, the present invention provides a method for forming a polymer composition comprising cells from a tissue sample, the method comprising:
providing a tissue sample comprising cells;
contacting the sample with the functionalised polymer described herein, or prepared by a method described herein, or a polymer composition comprising the functionalised polymer described herein, in binding conditions, said binding conditions being conditions that enable binding of cells in the sample to the functionalised polymer, so that said cells are bound to the functionalised polymer;
culturing the cells bound to the polymer under conditions and for a time that allows the cell number to increase;
providing conditions to induce a solid to liquid phase change of the functionalised polymer;
   thereby forming a polymer composition comprising cells from a tissue sample.

The present invention also provides a method for treating an individual comprising:
forming a composition according to a method described herein, or being provided with a composition formed a method described herein;
administering the composition to the individual, thereby treating the individual.

One particular example of an application of the functionalised polymer described herein is in methods for the provision of cells having chondrogenic potential to be used for repair or restoration of an articular surface. This method is now described further with reference to this specific implementation.

In a first step, the method comprises providing or having provided a tissue sample comprising cells. Generally, the sample is provided from the individual requiring treatment. It is a particular advantage of the method that it may be used in cell therapy and/or surgical techniques that are based on implementation of autologous cells.

A tissue sample may be obtained from tissue of the individual requiring treatment or may be taken from another individual. The tissue sample contains cells having the relevant function or the capacity to generate cells having the relevant function when (re) implanted into the individual. For example, the tissue sample contain cells with chondrogenic potential where the purposes is for use in producing cartilage (i.e. to treat a cartilage defect). In one embodiment, providing or having provided a tissue sample comprising cells does not involve a surgical step on a human or animal.

As used herein “chondrogenic potential” in the context of a cell means that the cell has the capacity to promote cartilage growth, particularly hyaline cartilage. This term is applied to cells which stimulate cartilage growth, such as chondrocytes, and to cells which themselves have the capacity to differentiate into a chondrocyte under appropriate conditions. Hyaline cartilage exists on the ventral ends of ribs, in the larynx, trachea, and bronchi, and on the articulating surfaces of bones.

A tissue sample that contains cells with chondrogenic potential may be a sample of adipose tissue. Adipose tissue contains adult stem cells which may be mesenchymal stem cells, or related precursors, or cells derived from these cells that have chondrogenic, osteogenic and/or adipogenic potential. Accordingly, the present invention provides methods for treating defects that require cells of chondrogenic, osteogenic or adipogenic potential. In other words, “a tissue sample” or “a tissue sample comprising cells” may be a tissue sample that comprises cells having chondrogenic, osteogenic and/or adipogenic potential. For example, methods of the invention and compositions produced therefrom (including cells isolated therefrom) could be used to treat bone defects, osteochondral defects, cartilage defects (not only articular cartilage), adipose tissue repair (e.g. breast reconstruction).

The mesenchymal stem cells, or related precursors, or cells derived from these cells have the capacity to form molecules of the extracellular matrix, and in particular molecules required for chondrogenesis and cartilage repair and restoration. Adipose derived stem cells (ADSCs) are particularly useful where the method is to be utilised in a procedure for cartilage repair or restoration. ADSCs may obtained from a number of different fatty tissues of the human or animal body. The ADSCs may be autologous or allogeneic.

In one particularly preferred embodiment, ADSCs are obtained from the infra patellar fat pad (IFP). The same tissue source (infrapatellar fat pad) can generate ADSCs that are known to display chondrogenic, osteogenic, and adipogenic potential. Given the 3 lineage differentiation potential, the cells could be used to treat bone defects, osteochondral defects, cartilage defects and adipose tissue repair.

An IFP may be obtained from an individual using standard techniques including those described herein. The IFP or sample therefrom may be harvested arthroscopically or upon open surgery. As described herein, an IFP generally comprises about 2 to 3 grams and about 8×10⁵ cells of which about 6×10⁵ cells are ADSC, therefore there are about 3×10⁵ ADSCs per gram of fat tissue in the IFP. Where a lesion has a greater volume, it may be necessary to utilise both or all fat pads, or to obtain ADSCs from other fat tissue. The inventors have found that about 5 million ADSC per ml of polymer (e.g. hydrogel) is required to repair or restore a cartilage lesion. In one embodiment, the step(s) of harvesting IFP include any as described herein.

In certain aspects of the invention, the method includes a step of isolating the cells from the extracellular matrix in the tissue sample. That isolation may be performed using one or more of mechanical disruption and enzymatic digestion, preferably both. For example, the IFP may be mechanically disrupted, minced or homogenized to isolate fat lobules. This can be achieved using a scalpel using standard techniques in sterile conditions within a few minutes. The purpose of the mechanical disruption is to improve exposure of the IFP to subsequent enzymatic digestion.

The disrupted IFP may then be subjected to collagenase digestion, the purpose of which is to separate the cells from extracellular matrix. Adipose tissue, including IFP generally contains a heterogeneous mixture of cells, in particular including blood cells, adipocytes, fibroblasts and ADSCs. Generally the collagenase is used at a specific activity of about 2 U/ml. This enables the digestion time to provide separated cells to be reduced to 85 minutes or less, preferably 45 minutes or less, preferably 30 minutes or less. Despite the teaching in the art, the inventors have found that such digestion does not impact on the viability or potential of cells of the IFP for chondrogenesis. The digestion may be performed in conditions where the mechanically disrupted tissue is agitated. In one embodiment, the step(s) of mechanical and/or enzymatic digestion include any as described herein.

At the completion of mechanical disruption and/or digestion, in certain aspects of the invention, the method includes separating the isolated cells from substantially all the fat and/or liquid present in the tissue sample. In this step the tissue sample may be the mechanically disrupted or enzymatically digested sample (or digest), and the sample or digest may be centrifuged to separate cells from a fat suspension and supernatant liquid. As defined herein the inventors have found that a cell pellet containing an appropriate number of cells for repair or restoration of an articular surface can be obtained in the cell pellet by centrifugation at 1000-2000 g for about 5-10 minutes, preferably 2000 g for 5 minutes. The centrifugation may be performed in the same vessel, i.e. tube, in which the mechanical disruption and/or enzymatic digestion occurred. In one embodiment, the step(s) of centrifugation include any as described herein.

The cell pellet thus formed contains a heterogeneous mixture of cells, including, as explained above, ADSCs and fibroblasts, and in addition, erythrocytes. Where the subsequent use of the composition formed by the method requires use of a composition that is devoid of erythrocytes, the cell pellet may be resuspended in buffer for lysis of red blood cells, filtered to separate debris from viable cells and further centrifugation for about 400-800 g for about 2-5 minutes, 5 minutes at about 400 g to obtain a cell pellet. The pellet may then be resuspended in medium to enable the pellet to be further processed to purify desired cells and remove unwanted cells.

The method also includes the step of contacting the tissue sample, isolated cells, digest or cell pellet that has been resuspended in medium as the case may be with the functionalised polymer in binding conditions, said binding conditions being conditions that enable binding of cells in the sample, isolated cells, digest or cell pellet to the functionalised polymer, so that said cells are bound to the functionalised polymer. As mentioned, most tissue biopsies or samples, whether required for autologous re-implantation or otherwise, will tend to contain more than one cell type of interest. In some autologous uses it is particularly important to separate a first cell type from a second or further cell type existing in a sample before re-implantation of the cells in the individual requiring the relevant treatment. An aspect of the method enables separation of cells of different phenotype on the basis of preferential or selective binding to a polymer substrate. The inventors have recognised that by contacting the cells of the tissue sample with a polymer under specific binding conditions it is possible to separate a first cell type from a second or further cell type i.e. to isolate a cell from a heterogeneous mixture of cells. Thus in one embodiment the method comprises the step of contacting the tissue sample, isolated cells or digest with the functionalised polymer in binding conditions, said binding conditions being conditions that enable the binding of cells to the functionalised polymer, and preferably to enable the binding of a first cell type to the functionalised polymer but not the binding of a second cell type to the functionalised polymer. In a subsequent method step the first cell type of interest may be separated from other unwanted cell types when the functionalised polymer having the first cell type bound thereto is separated from the 2nd, further or other cell types of the sample. In one embodiment, the step(s) of cell adherence to the functionalised polymer include any as described herein.

In one embodiment the step enables the binding of ADSCs to a polymer in conditions where other cells, and in particular, fibroblasts are unable to bind to the functionalised polymer. In this embodiment the polymer may comprises the following features: 1. Cellular adhesion, 2. Inducible phase change, preferably reversible phase change, 3. Crosslinkability, as described herein.

In a particularly preferred embodiment, the functionalised polymer is in contact with (i.e. non covalently bound or attached to) a solid phase, such as a surface of a particle, vessel or device. In this embodiment the functionalised polymer may form a continuous or interrupted polymer surface on the solid phase, thus providing a surface for cells to bind to.

Particularly preferred particles, vessels or devices include those that are routinely used in cell culture. For example, a particle may be a bead or nanoparticle. A vessel may be a dish, flask, tube or other vessel used in, or for, cell culture.

Where the solid phase is a particle, the particle may be a gold particle and the polymer may be coated thereon.

Thus, in one embodiment, the functionalised polymer is capable of attaching to a solid phase of a particle, vessel or device, or capable of forming a particle in the binding conditions.

In other embodiments, the particle is formed from the functionalised polymer described herein. The functionalised polymer may preferably comprise the following features: 1. Cellular adhesion, 2. Inducible phase change, preferably reversible phase change, 3. Crosslinkability, as described herein.

Typically the functionalised polymer for use in the method may form a hydrogel at room temperature, may be liquefied by a chelator chelating an ionic crosslinking agent or by heating to a temperature above room temperature that does not impact on the viability or function of ADSCs, and may be irreversibly cross linked, for example by visible light, UV radiation, enzymatic or electric field during or after re-implantation.

In any aspect or embodiment, the functionalised polymer is capable of reversible liquid-solid phase change. For example, the polymer may exist as a liquid, or semi-liquid, at room temperature and change to a solid, or semi-solid, by a change in temperature or in the presence of a chemical compound, for example a compound that can liberate divalent cations. Preferably the functionalised polymer has a reduction in flowability, e.g. has a phase change from a liquid to a solid, in the presence of an ionic crosslinking agent, for example a divalent cation such as Ca²⁺. Examples of suitable ionic crosslinking agents include calcium chloride (CaCl₂)), calcium sulfate (CaSO₄) and calcium carbonate (CaCO₃). In another embodiment, the functionalised polymer is capable of a solid to liquid phase change caused by a chelator chelating a divalent cation, for example the divalent cation that caused a liquid to solid phase change. The chelator may be any chelator capable of chelating an ionic crosslinking agent, for example a divalent chelator such as ethylenediaminetetraacetic acid (EDTA), ethylene glycol tetraacetic acid (EGTA), or citric acid.

The binding conditions may involve the contact of the cells of the sample with the functionalised polymer when the polymer is in a liquid state, a gel or a solid state.

An example of binding conditions that enable binding of cells in a sample to the functionalised polymer are as follows. The cell pellet that has been resuspended in medium may be cultured in, or on a vessel that contains one or more surfaces that have been coated with the functionalised polymer for selective or preferential adherence of stem cells or ADSCs. The functionalised polymer may be utilised at a concentration of from about 3% w/v to about 10% w/v, preferably from about 5% w/v to about 8% w/V. The cells are maintained in this environment for a time period that stem cells or ADSCs may preferentially adhere to the polymer. The non-bound cells may be removed, for example by washing, thereby separating the functionalised polymer with attached stem cells or ADSCs from the sample to form a composition in the form of cells bound to the functionalised polymer.

In any method of the invention, the functionalised polymer may be in the form of 3D particles that can be created manually (using a needle/syringe combination), with a microfluidic system or via inkjet 3D printing. 3D particles may be generated via crosslinking with 18-36 mM CaCl₂). An example of binding conditions that enable binding of cells in a sample to 3D particles are as follows. Cells and 3D particles may be seeded into a bioreactor at an appropriate cell: sphere ratio (e.g. 10 cells to every particle). 3D particles and sphere are used herein interchangeably. The bioreactor may be filled with tissue culture medium (TCM). Spinning intervals involving short spinning periods, followed by longer non-spinning periods, may be undertaken to ensure cell adhesion to the particles.

In any method of the invention, the functionalised polymer may be in the form of a layer that can be created by casting the functionalised polymer in a mould, for example as described herein.

In any embodiment, the polymer may be capable of irreversible crosslinking. Therefore, the polymer may be capable of reversible phase change, or reversible crosslinking, preferably mediated or caused by a chemical such as a divalent cation containing or liberating compound (i.e. ionic crosslinking), or by temperature changes, and may also be capable of irreversible crosslinking, preferably mediated or caused by exposure to light (i.e. is photocrosslinkable).

The method also includes a step of culturing the cells bound to the polymer under conditions and for a time that allows, or causes, an increase in cell number. Preferably, the conditions and time allows, or causes, at least 2 cycles of cell divisions, in other words allows a first division of the cells that initially adhere to the polymer, and then a division of the daughter cells from that first division. The conditions, such as tissue culture medium, will be known to the skilled person and relate to the specific cell type being expanded. There will be some variability in how quickly cell cultures expand and the number of cells on a random selection of polymer particles could be used to monitor the degree of expansion. However, after at least 5, 6 or 7 days in culture there should be at least 2 cycles of expansion of cells having chondrogenic potential. Therefore, an increase of about 3-4 times the original cell number should be present. Preferably, the culturing conditions and time allows an increase in number of stem cells, for example ADSCs. More preferably, the culture conditions allows an increase in cell number of stem cells and also priming of those stem cells to differentiate into a cell type of interest, for example, priming of ADSCs to form chondrocytes. Priming is performed on the same polymer without any passaging, thus continuing to avoid the use of any proteolytic agents such as trypsin. Accordingly, any method of the invention as described herein further includes a step of priming the cells at the same time or subsequently to culturing the cells that allows an increase in cell number.

In one embodiment, the step(s) of cell expansion on the polymer include any as described herein.

Bioreactor contents may be spun continually to allow for cell expansion whilst avoiding alginate sphere/disc agglomeration. In one embodiment, half of TCM volume is then removed and replaced with fresh TOM at an interval of 2-3 days. The protocol continues until the required amount of cells is reached.

The bioreactor is loaded with a ratio of cells and spheres, for example 10:1,000,000 cells and 100,000 3D particles. All of these particles are suspended in TOM within the bioreactor. The bioreactor may then be moved into an incubator and kept at 37° C., 5% CO2—it is placed upon a Cimerac magnetic stirring apparatus at this time. The reactor impellor is then subjected to an interval protocol (this is controlled by the magnetic stirrer); the impellor may spin at 50 RPM for 2 minutes, after which a non-spinning interval period of 30 minutes is enforced. This cycle of stirring/non-stirring periods is continued for 4 hours, as these spin breaks are essential to allow for cell adhesion to the spheres. Once the 4 hour protocol is completed, the impellor is then reverted back to the standard stirring protocol of continual 50 RPM stirring without interval.

The bioreactor is then filled with extra TOM, to a final volume of 50 or 100 mL (this allows for appropriate cell culture conditions when the impellor is spinning). At a time point of 48 hours following the end of the stirring interval period, a total of 50% of the TCM within the bioreactor may be removed and replaced with fresh TCM—this is undertaken without removing spheres. The media continues to be replaced every 2-3 days following the previous TCM replacement, until the cell culture protocol is completed.

Alternatively, the cells may be cultured or expanded on any particle, vessel or device described herein.

The method also includes a step of providing conditions to induce a phase change of the functionalised polymer. The phase change results in increase the flowability of the polymer. This can be achieved by heating the functionalised polymer (with the expanded cells still adhered) or by applying a chemical (e.g. EDTA) to reduce the degree of ionic cross-linking within the functionalised polymer (with the expanded cells still adhered). Any treatment may, but typically does not, reduce the adherence of the cells for the polymer.

In one embodiment of this step, the step comprises heating the polymer composition to melt the polymers, to increase the flowability of the composition, or to liquefy the polymers. The purpose of this step is generally to liberate or to release the polymer/cell complex from a solid phase to which the polymer is bound and/or to enable the polymer/cell complex to be administered in a cell therapy or surgical procedure by utilising the properties of flow of the melted or liquified composition, for example by extrusion, injection or 3D printing in the individual. In one exemplification of the method, the cells that remain bound to the polymer after the washing step described above may be subjected to heating by heating the vessel to which the functionalised polymer is bound to about 37° C., the result of which is to melt the hydrogel thereby forming a composition having the desired properties of flow from which the solid phase to which the functionalised polymer was earlier attached can be removed. Thus, it will be recognised that functionalised polymer substrates for use in the method enable separation of cells on a solid substrate and release of cells from the solid substrate without affecting the viability of the desired cells.

Where the functionalised polymer forms a particle, the heating of the polymer composition may liquefy the particle. In one embodiment, after the step in which a phase change is induced in the functionalised polymer, for example by heating, thereby liquifying the functionalised polymer, the cells that had bound to the functionalised polymer prior to the phase change induction remain bound to the functionalised polymer after the completion of the step. Thus, after a heating step, the composition does not become multiphasic, with for example, one phase containing functionalised polymer only and the other phase containing cells only. Instead, the cells remain bound to, or embedded in, the functionalised polymer after the heating step and this assists in the uniform delivery of the cells to a defect as the composition is administered during a re-implantation procedure.

In one particularly preferred embodiment the functionalised polymer has a melting temperature of below the temperature at which the desired functional properties of the cell of interest become compromised.

In a particularly preferred embodiment, the functionalised polymer selected for use in the method is one that is biocompatible with the individual and supports the cell in its delivery of the relevant cellular function when the cell is (re) implanted. This is advantageous as it enables the composition that has been heated to be utilised directly for re-implantation of the cells without further processing. As exemplified herein, the inventor has found that an alginate derived polymer is particularly useful because it can be directly injected into an articular defect or lesion and subsequently degrades enabling the release of ADSC for migration to the articular surface and chondrogenesis.

In another embodiment, a chelating agent such as EDTA is applied to increase the flowability of the functionalised polymer and which does not substantially affect the viability of the desired cells. This particularly applies to a functionalised polymer which has been reversibly crosslinked, or undergone a liquid to solid phase change, in the presence of an ionic crosslinking agent such as a divalent cation. The phase change also allows a step of mixing the cells with the liquefied polymer to be performed. This mixture can then be administered in a cell therapy or surgical procedure, particularly to an articular cartilage defect. Alternatively, the mixture may be stored for later use, for example in cellular banking. In one embodiment, the EDTA is at a concentration of equal to, or less than, 250 mM, equal to, or less than, 200 nM, equal to, or less than, 150 nM, equal to, or less than 100 nM, or equal to, or less than, 90 nM. Preferably, the EDTA is applied for up to about 30 minutes, preferably from about 5 minutes to about 15 minutes.

In one embodiment, the step(s) of phase change of the polymers include any as described herein.

In certain aspects of the invention, the method includes a step of administering the composition to an articular cartilage defect in the individual. The composition may be the flowable functionalised polymer cell combination. Alternatively, the composition may be a mixture or emulsion of the cells and the flowable functionalised polymer.

The composition may be delivered to the site of (re) implantation arthroscopically (with ultrasound or imaging guidance) or upon open surgery. The delivered composition may be hardened by the activation of a photoinitiator. The photoinitiator may be activated with visible light. An example of a suitable photoinitiator is lithium phenyl-2,4,6 trimethylbenzoylphosphinate (LAP). For example, the hydrogel may comprise the functionalised polymer (5-15 wt/vol %) and lithium phenyl-2,4,6 trimethylbenzoylphosphinate (LAP) 0.05% or 0.1%. This photocrosslinkable hydrogel may be cross-linked using conditions that are compatible with cell viability and chondrogenesis. Such conditions include 405 nm light source at 20 mW/cm² for 1 minute or 30 seconds.

In one embodiment, the step(s) of delivery include any as described herein.

The present disclosure also includes the following according to the numbered clauses:

1. A method for forming a cell composition from a tissue sample, the method comprising:
providing a tissue sample comprising cells;
contacting the sample with the functionalised polymer described herein or prepared by a method described herein, or the polymer composition comprising the functionalised polymer described herein, in binding conditions, said binding conditions being conditions that enable binding of cells in the sample to the functionalised polymer, so that said cells are bound to the functionalised polymer;
culturing the cells bound to the functionalised polymer under conditions and for a time that allows the cell number to increase;
providing conditions to induce a phase change of the functionalised polymer, wherein the cells remain bound to the phased changed functionalised polymer;
   thereby forming a cell composition from a tissue sample.

2. The method of clause 1, further comprising a step of isolating the cells from the extracellular matrix in the tissue sample.

3. The method of clause 2, wherein isolating the cells from the extracellular matrix is performed by mechanical disruption.

4. The method of clause 2, wherein isolating the cells from the extracellular matrix is performed by enzymatic digestion.

5. The method of any one of clauses 2 to 4, wherein isolating the cells from the extracellular matrix is performed by mechanical disruption and enzymatic digestion.

6. The method of any one of clauses 2 to 5, wherein isolating the cells separates the cells from any fat lobules in the sample.

7. The method of any one of clauses 4 to 6, wherein the enzymatic digestion is performed with collagenase.

8. The method of clause 7, wherein the collagenase is used at a specific activity of 2 U/ml for a period of 30 minutes or less.

9. The method of any one of clauses 2 to 8, further comprising the step of separating the isolated cells from substantially all the fat and/or liquid present in the sample.

10. The method of clause 9, wherein separating the isolated cells may be performed by centrifugation.

11. The method of clause 10, wherein the centrifugation is performed at about 2000 g for about 5 minutes to form a cell pellet.

12. The method of clause 11, wherein the cell pellet is resuspended in a buffer for lysis of red blood cells.

13. The method of clause 12, further comprising filtering the cells in the lysis buffer to separate debris from viable cells and further centrifugation for about 5 minutes at about 400 g to obtain a further cell pellet.

14. The method of clause 1, wherein the polymer is capable of attaching to a solid phase of a particle, vessel or device, or capable of forming a particle, in said binding conditions.

15. The method of any one of clauses 1 to 14, wherein the functionalised polymer is capable of binding to cells in said binding conditions that are human adipose derived stem cells (ADSCs) or hADSC precursor cells, or to cells that are derived from hADSC that are chondrogenic or that have chondrogenic potential.

16. The method of any one of clauses 1 to 15, wherein the functionalised polymer is not capable of binding to fibroblasts in said binding conditions.

17. The method of any one of clauses 1 to 16, wherein the cell adhesion moiety of the functionalised polymer binds to an extracellular matrix adhesion receptor.

18. The method of clause 17, wherein the extracellular matrix adhesion receptor is an integrin receptor.

19. The method of any one of clauses 1 to 18, wherein the cell adhesion moiety of the functionalised polymer comprises an integrin binding motif.

20 The method of clause 19, wherein the integrin binding motif is RGD.

21. The method of any one of clauses 1 to 20, wherein the functionalised polymer is capable of reversible liquid-solid phase change.

22. The method of any one of clauses 1 to 21, wherein the functionalised polymer is capable of a liquid to solid phase change caused by an ionic crosslinking agent.

23. The method of clause 22, wherein the ionic crosslinking agent is a divalent cation.

24. The method of clause 23, wherein divalent cation is Ca²⁺.

25. The method of any one of clauses 1 to 24, wherein the functionalised polymer is capable of a solid to liquid phase change caused by a chelator chelating an ionic crosslinking agent.

26. The method of clause 25, wherein the chelator is EDTA.

27. The method according to any one of clauses 1 to 26, wherein the functionalised polymer is capable of photo-crosslinking.

28. The method of any one of clauses 1 to 27, wherein the tissue sample comprises a first cell type and a second cell type and wherein the binding conditions enable the binding of the first cell type to the functionalised polymer and wherein the binding conditions do not enable binding of the second cell type to the functionalised polymer.

29. The method of clause 28, wherein separation of the functionalised polymer from the tissue sample forms a cell composition consisting of cells of the first cell type, and forms a waste stream comprising cells of the second cell type.

30. The method of clauses 28 or 29, wherein the first cell type is a hADSC or chondrogenic cell and the second cell type is a fibroblast.

31. The method of any one of clauses 1 to 30, wherein the tissue sample is obtained from the infrapatellar fat pad.

32. The method of clause 31, wherein the fat pad has a weight of about 2 to 3 g.

33. The method of any one of clauses 1 to 32, wherein the functionalised polymer is in the form of a 3D particle.

34. The method of clause 33, wherein the sample is contacted with the 3D particle in a bioreactor at a cell:particle ratio of about 10 cells to every particle.

35. The method of any one of clauses 1 to 34, wherein the step of culturing the cells allows at least 2 cycles of cell divisions.

36. The method of any one of clauses 1 to 34, wherein the step of culturing the cells is for a period of at least 5, at least 6 or at least 7 days.

37. The method of any one of clauses 1 to 34, wherein the step of culturing the cells results in an increase of about 3-4 times the original cell number.

38. The method of any one of clauses 1 to 34, wherein the step of culturing the cells results in about 5 million cells.

39. The method of any one of clauses 1 to 38, wherein when the tissue sample contains stem cells, preferably ADSCs, the method further comprises the step of priming of those stem cells to differentiate into a cell type of interest, for example, priming of ADSCs to form chondrocytes.

40. The method of clause 39, wherein the priming step occurs at the same time or subsequent to culturing the cells that allows an increase in cell number.

41. The method of any one of clauses 1 to 40, wherein the conditions to induce a phase change of the functionalised polymer is heating.

42. The method of any one of clauses 1 to 41, wherein the conditions to induce a phase change of the functionalised polymer is application of a chelator.

43. The method of clause 42, wherein the chelator is EDTA.

44. The method of any one of clauses 41 to 43, wherein the phase change increases the flowability of the functionalised polymer enabling the cell composition to be administered to an individual at room temperature by injection, extrusion or 3D printing.

45. The method of clause 41, wherein the heating step comprises heating the cell composition to a temperate that does not affect the viability of the cells in the cell composition.

46. The method of any one of clauses 1 to 45, wherein the functionalised polymer has a melting temperature of about 25 to about 30° C.

47. A method for treating an individual comprising:
forming a cell composition according to any one of the preceding clauses, or being provided with a cell composition formed according to any one of the preceding clauses;
administering the cell composition to the individual, thereby treating the individual.

48. The method of clause 47, wherein the cell composition is formed from a tissue sample obtained from the individual.

49. The method of clause 48, wherein the cell composition is formed from a tissue sample obtained from an infrapatellar fat pad of the individual.

50. The method of any one of clauses 47 to 49, wherein the cell composition is administered by injection, extrusion or 3D printing.

51. The method of any one of clauses 47 to 50, wherein the individual has a condition of an articular surface requiring repair or restoration.

52. The method according to any one of clauses 47 to 51, wherein the cell composition is administered to an articular surface requiring repair or restoration.

53. The method according to any one of clauses 47 to 52, wherein the cell composition is administered arthroscopically, preferably with ultrasound or imaging guidance.

54. The method according to any one of clauses 47 to 53, wherein the cell composition is administered upon open surgery.

55. The method according to any one of clauses 47 to 54, wherein the delivered cell composition is hardened by the activation of a photoinitiator.

56. The method of clause 55, wherein the photoinitiator is activated with visible light.

57. The method of clause 56, wherein the photoinitiator is LAP.

58. The method of clause 56 or 57, wherein a 405 nm light source at 20 mW/cm² is applied for 1 minute or 30 seconds.

59. A cell composition obtained by a method of any one of clauses 1 to 46.

60. A cell composition obtainable by a method of any one of clauses 1 to 46.

61. Use of a cell composition of clause 59 or 60 in the manufacture of a medicament for treatment of a condition requiring (re)-Implantation of cells for said treatment.

62. A cell composition of clause 59 or 60 for use in the treatment of a condition requiring implantation of cells for said treatment.

63. A cell composition of clause 59 or 60 when used for treatment of a condition requiring implantation of cells for said treatment.

64. A kit for use, or when used, in a method of any one of clauses 1 to 58, the kit comprising a polymer as described herein.

65. The kit of clause 64, further comprising written instructions to perform a method of any one of clauses 1 to 58.

### EXAMPLES

The invention will be further described by way of non-limiting examples. It will be understood to persons skilled in the art of the invention that modifications may be made without departing from the spirit and scope of the invention.

### Example 1. Synthesis of Universal Polymer Alg-MA-RGD

### Materials

Alginate was sourced from primary manufacturers (SNAP Natural & Alginate Products Pvt. Ltd. and KIMICA Corporation). The alginate from each source was characterised (tested for viscosity, 1H NMR, molecular weight by gel permeation chromatography, and calcium crosslinking) and was used without purification. Methacrylic anhydride (MA) (94%) stabilized with topanol inhibitor was purchased from Sigma-Aldrich. Phosphate buffered saline (PBS) reagents and sodium hydroxide pellets PBS were purchased from Sigma Aldrich. Peptide CRGDS (≥95% pure by HPLC) was purchased from Mimotopes Pvt. Ltd.

### Synthesis of Alg-MA

Alginate methacrylate (Alg-MA) was synthesised following the method reported by Mignon et al (Carbohydrate Polymers, Vol 155, 2 Jan. 2017, pages 448-455), with precise control of pH during methacrylate functionalisation, temperature and also limited light exposure during the entire process of reaction, purification and storage. The synthetic scheme for the methacrylate functionalisation of alginate is shown in Scheme 3.

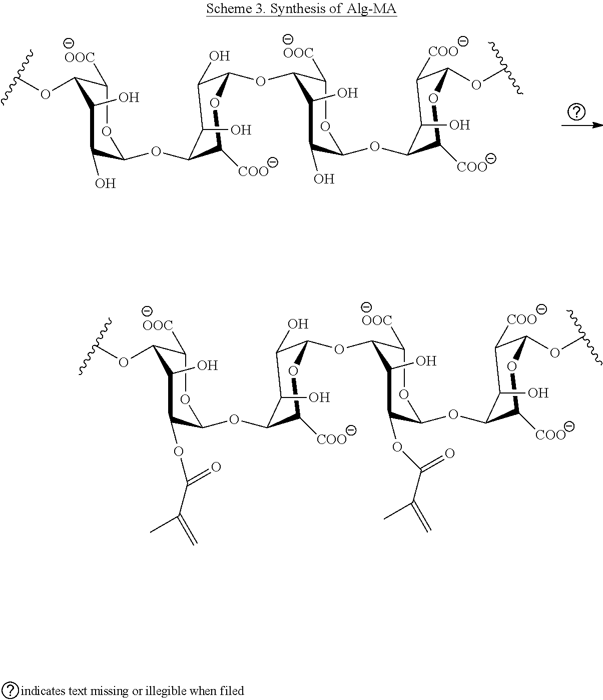

Briefly, hydroxyl groups of alginate were functionalised by dissolving 40 g of alginate (MW 270 kDa, M/G ratio 1.3; or MW 280 kDa, M/G ratio 0.64) in Milli-Q water at 2-2.5% concentration using mechanical stirrer at 50° C. Once the alginate was dissolved, the temperature was reduced to 22-25° C. and methacrylic anhydride (MA) was added, typically 2-5% w/w of alginate (depending on degree of functionalisation to be achieved) at 20-25° C. over 12-16 hours. During the reaction and on addition of MA, methacrylic acid was released which lowers the pH of the reaction. To facilitate the functionalisation, the acidity of the mixture was constantly monitored and the pH was increased to 7-8 by adding 5 M sodium hydroxide (NaOH) solution incrementally in small quantities over the course of reaction. The pH was not allowed to exceed pH 9 to avoid hydrolysis of the ester in the reaction product. The reaction was continued at room temperature for a further 12-14 hours. Finally the reaction was stopped and pH was adjusted to ˜7 by addition of dilute sodium hydroxide.

The Alg-MA was purified using a 8 kDa MW cut-off dialysis membrane. Temperature was maintained at room temperature throughout the purification. The solution of Alg-MA after purification was a clear colourless viscous liquid. The pH was adjusted to 7.0±0.3 by addition of dilute sodium hydroxide. The viscous solution of Alg-MA was then freeze-dried to give the product as a bright white solid in 65-70% yield. Impurities in the final product were determined by NMR to be low (no significant peak of impurity left after purification). 1H NMR confirmed successful functionalisation of Alg to Alg-MA, characterised by new peaks appearing at 1.9 ppm, 5.8 ppm and 6.2 ppm corresponding to the MA group. The molecular weight of each product was determined by GPC. The degree of functionalisation of each product was calculated by 1H NMR in D₂O (water suppression conditions) based on integration of the two peaks of methacrylate groups appearing at 5.8 and 6.2 ppm. The degree of functionalisation for the products was found to vary between 11-88%, based on the number of functionalised alginate monomer units, depending on the access of MA, addition rate of MA and pH during the course of reaction.

### Synthesis of Alg-MA-RGD

Alg-MA was functionalised with CRGDS (RGD peptide) via thiol-Michael addition reaction to provide Alg-Ma-RGDS as shown in Scheme 4. Precise control of pH during the reaction allowed grafting of the RGD peptide to the methacrylate functionality.

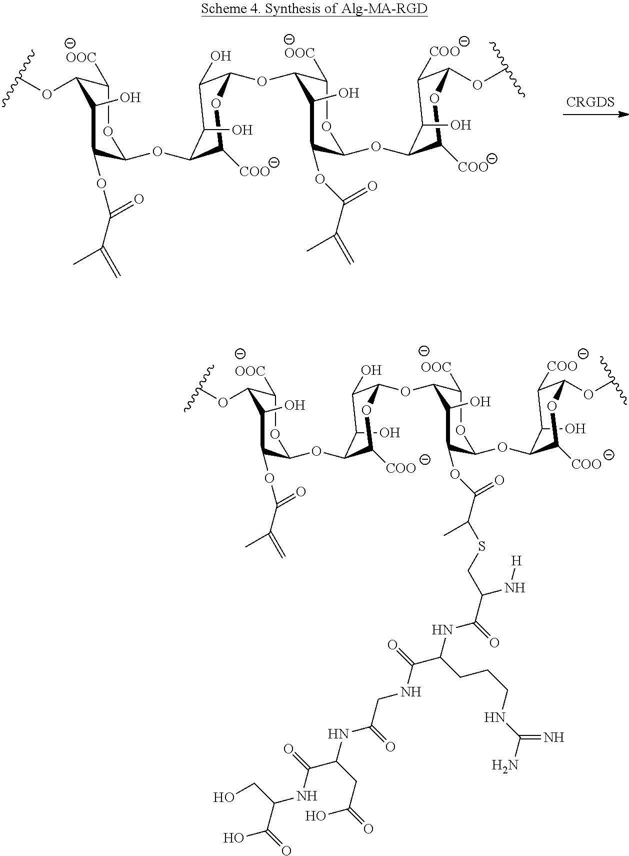

Briefly, Alg-MA (1-2 g) was dissolved in sodium carbonate/sodium bicarbonate buffer and the pH adjusted 9.0-9.5 using dilute sodium hydroxide. Quantities of RGD peptide suitable to provide 5-15% substitution of MA was added and the reaction stirred for 12-14 hours to complete the reaction. During the reaction the reaction contents were protected from light and the pH was maintained at 9.5±0.2 by addition of dilute sodium hydroxide. The reaction contents were then subjected to dialysis using 0.8-3 kDa membrane to remove any unreacted RGD peptide and buffering salts. The reaction contents were freeze-dried while protecting from light to provide the final product. 1H NMR confirmed successful functionalisation of the MA groups of Alg-MA to Alg-MA-RGD, characterised by new peaks appearing within 1.2 ppm to 4.0 ppm corresponding to the RGD peptide. These samples were found to have different NMR spectra to samples containing a mixture of Alg-MA and RGD peptide. Elemental analysis also confirmed presence of the product, characterised by an increase in sulfur and nitrogen percentages compared to starting Alg-MA. The product was tested for degree functionalisation by elemental analysis, based on the elemental composition of the Alg-MA-RGD product after dialysis purification compared to a control sample containing a mixture of Alg-MA and RGD peptide. The elemental compositions of various samples are provided in Table 1.

TABLE 1
Elemental compositions of Alg-MA-RGD products and controls

| Sample | N % | C % | H % | S % | RGD DOF |
|---|---|---|---|---|---|
| Alg-MA- | 0.80 | 28.77 | 4.87 | 0.23 | 5% |
| RGD | | | | | |
| prepared | | | | | |
| from Alg-MA | | | | | |
| 40% MA | | | | | |
| Alg-MA- | 1.58 | 32.55 | 4.55 | 0.4 | 8% |
| RGD | | | | | |
| prepared | | | | | |
| from Alg-MA | | | | | |
| 60% MA | | | | | |
| Alg-MA- | 2.62 | 31.17 | 4.69 | 0.73 | 14% |
| RGD | | | | | |
| prepared | | | | | |
| from 60% | | | | | |
| MA | | | | | |
| Alg-MA with | 0.00 | 30.38 | 5.47 | 0.00 | — |
| 44% MA | | | | | |
| DOF | | | | | |
| Alg-MA + | 1.86 | 34.83 | 5.09 | 0.50 | Equivalent |
| RGD | | | | | to 10% |
| peptide | | | | | |
| (control with | | | | | |
| 9.8% RGD | | | | | |
| peptide by | | | | | |
| physical | | | | | |
| mixing) | | | | | |

### Example 2. Properties of Alg-MA

Properties of various Alg-MA samples upon calcium crosslinking and photocrosslinking were assessed. Storage modulus may be used as an indicative proxy used to keep within a certain bounds of handling and functionality as determined by the required application. Examples of suitable parameters include the following:
for the ionic crosslinking step, a storage modulus of at least 5 kPa (and preferably >10 kPa) may produce a material which can be easily handled and which forms a stable substrate for cell culture over 7 days (i.e. to mitigate risks of rapid degradation in culture);
for the ionic crosslinking step, a storage modulus of less than 50 kPa may be preferred as higher stiffness materials are more difficult/slower to decrosslink in the subsequent phase change step;
for the photocrosslinking step, a storage modulus of at least 5 kPa may produce a material which can hold its shape when cured in situ in a cartilage defect;
for the photocrosslinking step, a storage modulus of no higher than 50 kPa may provide an environment soft enough to allow chondrogenesis.

Calcium crosslinking and photocrosslinking tests were carried using a TA instrument rheometer. For calcium crosslinking studies, Alg-MA solutions were prepared at 5% (w/v) concentration of Alg-MA sample in PBS. 50 μL of each sample was loaded on the rheometer. At 50 second time point CaCl₂) (0.8 mL, 1M) was added onto sample. Sample was left to crosslink for 15 minutes at room temperature and increase in storage modulus is tested over a period of 10 minutes. For photocrosslinking studies, Alg-MA solutions were prepared at 5% (w/v) concentration of Alg-MA sample in PBS containing 0.1% (w/V) photo initiator (LAP). 100 μL of each sample was loaded on the rheometer. At 100 second time point, the UV light was turned on (320-500 nm, 20 mW/cm²) and the sample was left to crosslink over a period of 300 seconds. Rheology parameters: strain: 1%, frequency: 10 rad/s, temperature: RT, geometry: cone, 12 mm (smaller geometry used to reduce the amount of sample tested). Alg-MA samples prepared from different alginate sources and having different degrees of methacrylate functionalisation (MA DOF) were assessed as shown in Table 2. In this study, Alg-MA samples having between ˜40-60% methacrylate functionalisation were found to have suitable calcium crosslinking and photorheology properties. It was hypothesised Alg-MA with 40-60% methacrylate functionalisation may also contain sufficient methacrylate groups that would allow for varying degrees of functionalisation with RGD peptide. Accordingly, the universal polymer prepared from Alg-MA having between 40-60% methacrylate functionalisation was investigated in further studies.

TABLE 2
Summary of properties of Alg-MA samples

| | | Storage modulus | |
|---|---|---|---|
| Alginate | MA | Ca²⁺ crosslinked (kPa as | |
| source | DOF | 5%) | Photocrosslinked (kPa) |
| 270 kDa, | 11% | Not tested | 5 |
| M/G ratio | 19% | Not tested | 8 |
| 1.30 | 25% | 40 | 22 |
| | 44% | 35 | 23 |
| | 60% | 34 | 24 |
| | 88% | 4 | 31 |
| 280 kDa, | 17% | 113 | 8.7 |
| M/G ratio | 54% | 72 | 28.6 |
| 0.64 | | | |

### Example 3. Storage Modulus Properties of Alg-MA and Alg-MA-RGD

The storage modulus (stiffness) on cationic crosslinking and photorheology of Alg-MA (methacrylation DOF 40% and 60%) and Alg-MA-RGD (RGD peptide functionalisation 5-15%) were measured. For calcium crosslinking studies, Alg-MA and Alg-MA-RGD solutions were prepared at 5% (w/v) concentration of Alg-MA/Alg-MA-RGD sample in PBS. 4×50 uL of each sample (200 μL in total) was cast and loaded on the rheometer. At 50 second time point CaCl₂) (2 mL, 180 mM) was added onto sample. Sample was left to crosslink for 15 minutes at room temperature and increase in storage modulus was tested over a period of 10 minutes. CaCl₂ was then removed and the samples rinsed with PBS. For decrosslinking studies, ionically crosslinked scaffolds were decrosslinked using EDTA (200 UL, 0.5 M and pH=8.5) after 1 hour at 37° C. using a warm water bath. For photocrosslinking studies, Alg-MA solutions were prepared at 5% (w/v) concentration of Alg-MA sample in PBS containing 0.1% (w/V) photo initiator (LAP). 100 uL of each sample was loaded on the rheometer. At 100 second time point, the UV light was turned on (320-500 nm, 20 mW/cm²) and the sample was left to crosslink over a period of 300 seconds. Rheology parameters: strain: 1%, frequency: 10 rad/s, temperature: RT, geometry: cone, 12 mm (smaller geometry used to reduce the amount of sample tested). Results are shown in FIG. 2 and Table 3.

TABLE 3
Storage modulus of Alg-MA and Alg-MA-RGD
upon calcium- and photocrosslinking

| | | | EDTA |
|---|---|---|---|
| | | | chelation |
| | Storage | Storage | and storage |
| | modulus | modulus | modulus on |
| | on photo- | on calcium | photo- |
| | crosslinking | crosslinking | crosslinking |
| Sample | (kPa) | (kPa) | (kPa) |
| Alg-MA with 40% | 23 | 35 | Not analysed |
| MA DOF | | | |
| Alg-MA-RGD | 12 | 14.8 | 1.1 |
| prepared from Alg- | | | |
| MA 40% MA DOF | | | |
| with 5% RGD | | | |
| peptide DOF | | | |
| Alg-MA with 60% | 24 | 34 | Not analysed |
| MA DOF | | | |
| Alg-MA-RGD | 13 | 24 | 1.5 |
| prepared from Alg- | | | |
| MA 60% MA DOF | | | |
| with 5-7% RGD | | | |
| peptide DOF | | | |
| Alg-MA-RGD | 7 | 13 | 1.1 |
| prepared from Alg- | | | |
| MA 60% MA DOF | | | |
| with 15% RGD | | | |
| peptide DOF | | | |

### Example 4. Formulations of Alg-MA-RGD

Alg-MA prepared from alginate source 270 kDa, M/G 1.3 and having ˜40% or ˜60% methacrylate functionalisation were used to prepare Alg-MA-RGD samples having different degrees of RGD peptide functionalisation. Formulations of Alg-MA-RGD were prepared by dissolving an appropriate amount of Alg-MA-RGD in an appropriate volume of PBS to provide a desired formulation concentration as summarised in Table 4. In these studies, formulations having a concentration of 5% or 8% w/v Alg-MA-RGD were found to be castable. Castable refers to the ability to retrieve a controlled amount of the material through a syringe or a pipette and to extrude the said amount in a vessel. The Alg-MA-RGD formulations found to be non-castable were too viscous to be manipulated.

TABLE 4
Summary of properties of Alg-MA-RGD formulations

| | | Formulation | |
|---|---|---|---|
| Alg-MA starting | RGD peptide | concentration | |
| material | DOF | (weight/vol) | Observations |
| Alg-MA | 5% | 5% | Formulation |
| with 40% | | | castable. |
| MA DOF | | 8% | Formulation |
| | | | castable. |
| | | 10% | Formulation not |
| | | | castable. |
| | 15% | — | Alg-MA-RGD not |
| | | | photocrosslinkable. |
| Alg-MA with 60% | 5% | 5% | Formulation |
| MA DOF | | | castable. |
| | | 8% | Formulation |
| | | | castable. |
| | | 10% | Formulation not |
| | | | castable. |
| | 15% | 5% | Formulation |
| | | | castable. |
| | | 8% | Formulation |
| | | | castable. |
| | | 10% | Formulation not |
| | | | castable. |

TABLE 5
Summary of % of functionalisation of photocrosslinkable
moiety (Methacrylation) of Alginate (Alg-Ma).
DOF = degree of functionalisation

| | Alg-MA | | |
|---|---|---|---|
| Alginate source | Batch No. | DOF | Observations |
| Mol. wt. 270k | #418 | 88% | Poor Ca²⁺ crosslinking as most |
| *(M/G: 1.30), | | | carboxylic functionalities |
| | | | were modified |
| | | | with methacrylate groups |
| | #428 | 25% | Alginate-MA was taken for |
| | | | functionalisation with |
| | | | CRGDS but did not |
| | | | have desired rheological |
| | | | properties and not |
| | | | enough methacrylate |
| | | | functionalities for |
| | | | RGD functionalisation (5-15%) |
| | #436 | 19% | Not the desired photo-rheology |
| | #438 | 11% | Synthesis parameter optimisation. |
| | | | Not the desired photo-rheology |
| | #440 | 44% | Appropriate Photorheology and |
| | | | Ca²⁺ crosslinking |
| | | | properties and could |
| | | | have enough methacrylate groups for |
| | | | varying degree of |
| | | | RGD functionalisation |
| | #454 | 60% | Appropriate Photorheology and |
| | (Similar to | | Ca²⁺ crosslinking |
| | D1) | | properties and could |
| | | | have enough methacrylate groups for |
| | | | varying degree of |
| | | | RGD functionalisation |
| Mol. wt. 280k | #AK02/91 | 17% | Poor photorheology and higher |
| *(M/G: 0.64) | | | stiffness of Ca²⁺ |
| | | | crosslinked structure that |
| | | | was difficult for chelation |
| | | | using the desired |
| | | | concentration of EDTA |
| | #AK2- | 54% | Appropriate Photorheology and |
| | 102 | | Ca²⁺ crosslinking |

TABLE 6
Summary of % of cell adhesion moiety (RGD) of Alginate (Alg-MA-
RGD). DOF = degree of functionalisation. Batches used for
sequential process analysis of the patent highlighted in green

| | Alg-MA-RGD | Alg-MA | RGD | |
|---|---|---|---|---|
| Alginate source | Batch No. | Initial DOF | functionalisation | Observations |
| Mol. wt. 270k | #432A | 25% | Low | Conditions were same |
| *(M/G: 1.30), | | | RGD | as reported by Sun et. Al. |
| | | | functionalisation | DOI: 10.139/C7TB01458A. |
| | | | | RGD functionalisation was |
| | | | | lower and impurities were high |
| | | | | as observed by NMR. Poor |
| | | | | rheological properties for |
| | | | | sequential process. |
| | #432 | 25% | ~6% | Improved reaction |
| | | | | conditions for thiol-Michael |
| | | | | addition, poor rheological |
| | | | | properties for sequential |
| | | | | process. |
| | #433 | 25% | ~5% | Improved reaction |
| | | | | conditions for thiol-Michael |
| | | | | addition, poor rheological |
| | | | | properties for sequential |
| | | | | process. |
| | #446 | 44% | 5% | Appropriate |
| | | | | Photorheology and Ca²⁺ |
| | | | | crosslinking properties and |
| | | | | could have enough methacrylate |
| | | | | groups for varying degree of |
| | | | | RGD functionalisation, tested |
| | | | | for sequential process. |
| | #456 | 60% | 7% | Appropriate |
| | | | | Photorheology and Ca²⁺ |
| | | | | crosslinking properties and |
| | | | | could have enough methacrylate |
| | | | | groups for varying degree of |
| | | | | RGD functionalisation, tested |
| | | | | for sequential process. |
| | #447 | 44% | 15% | Poor rheological |
| | | | | properties for sequential |
| | | | | process. |
| | #457 | 60% | 15% | Appropriate |
| | | | | Photorheology and Ca²⁺ |
| | | | | crosslinking properties and |
| | | | | could have enough methacrylate |
| | | | | groups for varying degree of |
| | | | | RGD functionalisation, tested |
| | | | | for sequential process. |

The formulations in Table 7 were similarly prepared and assessed in subsequent studies. It will be appreciated that the weight of Alg-MA-RGD in the formulations may be different from the final solid content present in a hydrogel prepared from the formulation.

TABLE 7
Formulations of Alg-MA-RGD

| Formulation No | Alg-MA-RGD sample | Concentration (wt/vol) |
|---|---|---|
| 1 | Alg-MA-RGD prepared from Alg-MA | 5 |
| | 40% MA DOF with 5% RGD peptide | |
| | DOF | |
| 2 | Alg-MA-RGD prepared from Alg-MA | 8 |
| | 40% MA DOF with 5% RGD peptide | |
| | DOF | |
| 3 | Alg-MA-RGD prepared from Alg-MA | 5 |
| | 60% MA DOF with 5% RGD peptide | |
| | DOF | |
| 4 | Alg-MA-RGD prepared from Alg-MA | 8 |
| | 60% MA DOF with 5% RGD peptide | |
| | DOF | |
| 5 | Alg-MA-RGD prepared from Alg-MA | 5 |
| | 60% MA DOF with 15% RGD peptide | |
| | DOF | |
| 6 | Alg-MA-RGD prepared from Alg-MA | 8 |
| | 60% MA DOF with 15% RGD peptide | |
| | DOF | |

### Example 5. Cell Culture Substrate Properties of Alg-MA-RGD Formulations

The ability of the universal polymer to form an ionically crosslinked network that could function as a substrate for cell culture was investigated. The formulations in Table 7 were assessed.

First, the adhesion of stem cells on calcium crosslinked Alg-MA-RGD was assessed. To prepare calcium crosslinked samples (Alg-MA-RGD layers), Alg-MA-RGD with varying degrees of MA and RGD peptide functionalisation were dissolved at the desired concentration (e.g. 5%-8%) in PBS 1× with 100 μg/ml Penicillin/Streptomycin (Gibco) overnight with stirring at 37° C. 15×1 mm silicone rubber cylindrical mould (180 UL volume) were placed on a nitrocellulose sheet pre embedded with 200 mM CaCl₂ solution in a 100 mm plastic dish. The Alg-MA-RGD formulations were casted into the cylindrical moulds. A second 200 mM CaCl₂ nitrocellulose sheet was placed on top of the mould and pressed with a 100 mm plastic dish cover. Cationic crosslinking was maintained for 15 min at room temperature. After cationic crosslinking the Alg-MA-RGD layer was transferred with a tweezer into a 24 low attachment plate, rinsed with PBS and incubated for 48 hours in adipose derived stem cells (ADSC) growing media [low glucose DMEM (Sigma-Aldrich) supplemented with 10% FBS (Gibco), 100 U/ml Penicillin and 100 μg/ml Streptomycin solution (Gibco), 2 mM L-Glutamine (Gibco), 15 mM HEPES (Gibco), 20 ng/ml epidermal growth factor (EGF) and 1 ng/ml fibroblast growth factor (FGF) (R&D Systems Inc., Minneapolis, MN, USA)] at 37° C. and 0.5% CO2.

Human Adipose Derived Stem cells (hADSCs) were isolated from human infrapatellar fat pad (IPFP) obtained intraoperatively from total knee arthroplasty with informed consent and approval. Patients with mild/severe osteoarthritis (OA) were recruited. Use of all human samples and procedures in this study was approved by the Human Research Ethics Committee Research Governance Unit of St. Vincent's Hospital, Melbourne, Australia [HREC/16/SVHM/186] and all experiments were performed in accordance with relevant guidelines and regulations. hADSCs were isolated and expanded as previously described by Ye et al (PLOS One. 2014 Jun. 11; 9 (6): e99410; erratum in: PLOS One. 2014; 9 (7): e102638). Briefly, the fat was diced using a sterile scalpel and digested with 0.1% Collagenase type II (Worthington Biochemical Corporation, Lakewood, NJ, USA) for 3 h at 37° C. under constant agitation, filtered through 100 μm cell strainer nylon (BD Falcon) and centrifuged at 400 g at room temperature for 5 min to separate the stromal fraction from the floating adipocytes. The supernatant was discarded, the cell pellet was resuspended in Red Cell Lysis Buffer (Sigma-Aldrich) and incubated at room temperature for 10 min. The lysate was centrifuged at 400 g at room temperature for 5 min and filtered through a 40 μm nylon cell strainer (BD Falcon). The isolated cells were then plated in monolayer culture on the Alg-MA-RGD layer or in plastic flasks.

1.6×10⁵ cell/cm² hADSCs isolated from infrapatellar fat pad were seeded onto the layers of Alg-MA-RGD and let grow for 7 days in 1 ml of Adipose Derived Stem Cells Growth Media [low glucose DMEM (Sigma-Aldrich) supplemented with 10% FBS (Gibco), 100 U/ml Penicillin and 100 μg/ml Streptomycin solution (Gibco), 2 mM L-Glutamine (Gibco), 15 mM HEPES (Gibco), 20 ng/ml epidermal growth factor (EGF) and 1 ng/ml fibroblast growth factor (FGF) R&D Systems Inc., Minneapolis, MN, USA].

The results of the cell adhesion study are shown in Table 8. An overall % of adhesion of greater than 90% was scored for all the materials tested, with Formulation 6 was found to have the highest % adhesion. A plastic tissue culture plate was used as a control.

TABLE 8
Adhesion of hADSCs on Alg-MA-RGD layers

| Sample | % Cell Adhesion^{a} |
|---|---|
| Plastic (control) | 100 |
| Formulation 1 | 93.5 |
| Formulation 2 | 95.1 |
| Formulation 3 | 92.4 |
| Formulation 4 | 95.8 |
| Formulation 5 | 93.8 |
| Formulation 6 | 98.6 |

^{a}Percentage adhesion calculated according to the following equation:

$$\% adhesion = ( \frac{Seeded Cells - Unattached Cells}{Seeded cells} ) \times 100$$

The ability of the calcium crosslinked Alg-MA-RGD to allow for cell proliferation was also assessed by conducting a metabolic activity assay over 7 days in culture. The metabolic activity was measured according to the following protocol at days 1, 3 and 7. CellTiter-BlueÆReagent (Promega, Madison, WI, USA) was used according to manufacturer's instructions. A total volume of 1.0 mL was used per sample. The cellular scaffolds were incubated for 3 h at 37° C. 5% CO2 with the CellTiter solution diluted in cell culture media and the solution collected and measured in a CLARIOStar plate reader at 550+/−15 excitation nm and 600+/−20 nm emission using the same gain for all readings.

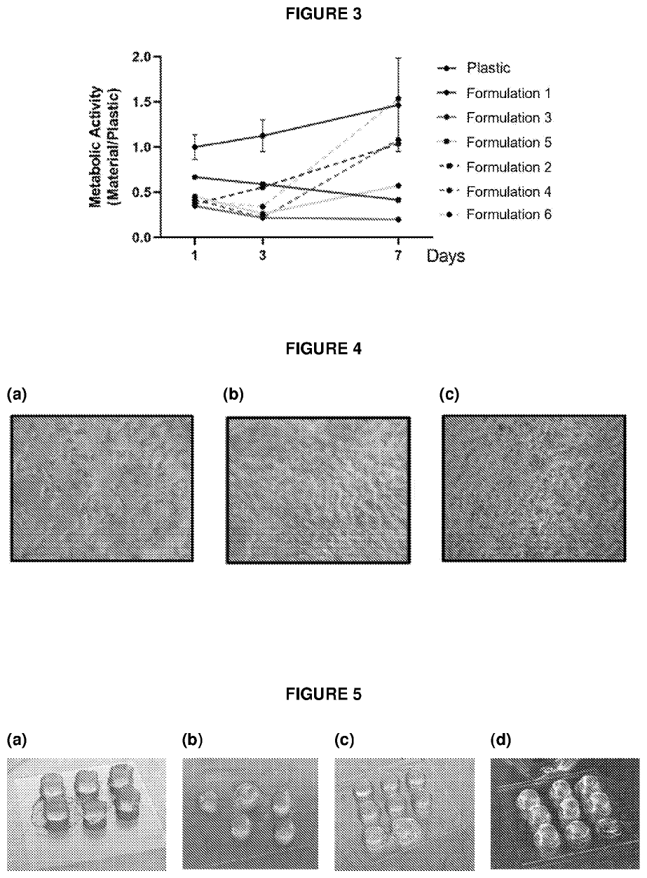

The results of the metabolic activity assay are shown in FIG. 3. The formulations having 8 w/v % concentration (Formulations 2, 4 and 6) were found to have comparable fold change increase in metabolic activity to the plastic control. Phase contrast images of cells at days 1, 3 and 7 for Formulation 6 were obtained using an EVOS FL imaging system with an EVOS 4× objective 0.13NA and are shown in FIG. 4.

The above results demonstrate that the universal polymer can form an ionically crosslinked network capable of functioning as a substrate for cell culture.

### Example 6. Phase Change Properties of Alg-MA-RGD Formulations

The ability of calcium crosslinked Alg-MA-RGD to undergo phase change to liquid was assessed. To fit within typical cell culture processes, the calcium crosslinked polymer would ideally be capable of liquefying within a timeframe of 5-15 minutes, which is the time frame required for standard enzymatic processes (e.g. Trypsin/EDTA 0.25%) to detach cells from plastic tissue culture systems.

The phase change studies may be performed according to the following procedure. Calcium crosslinked Alg-MA-RGD layers are prepared according to the procedure in Example 5 without cell seeding. The Alg-MA-RGD layers have a volume of 180 μL. The growth media is removed and 60 μL of 0.5-250 mM EDTA solution is added (volume ratio of Alg-MA-RGD:EDTA 3:1; this was found to be a suitable ratio that limited dilution of the polymer for the subsequent photocrosslinking step). Phase change (liquification) is performed at 37° C. 5% CO2 (cell culture incubator) for 15 minutes. The liquification time may be quantified by an acellular observation test.

### Example 7. Bioscaffold Properties of Alg-MA-RGD Formulations

The ability of the phase changed (liquified) Alg-MA-RGD formulations (bioinks) to function as a bioscaffold after delivery and photocrosslinking was investigated. As reported by Onofrillo et al (Biomaterials, Volume 264, January 2021, 120383), to be useful as a bioscaffold the polymer would ideally be capable of retaining shape after implantation, retaining cell viability and reaching an achievable storage modulus (stiffness) between 7+/−4 and 37+/−6 kPa after photocrosslinking to allow cell differentiation for tissue engineering applications such as cartilage regeneration. In these studies, control gelatin methacrylate (GelMA) 6% was used and evaluated as a gold standard for chondrogenesis in bioscaffolds.

The cell culture samples from Example 5 were subjected to phase change at day 7 according to the following procedure. The growth media was removed from the samples and 60 μl of 250 mM EDTA solution was added (volume ratio of Alg-MA-RGD:EDTA 3:1). Phase change (liquification) was performed at 37° C. 5% CO2 (cell culture incubator) for 15 minutes to provide a bioink. After phase change, the obtained bioink was resuspended with a pipette by mixing the bioink up and down with a pipette. The bioink was then transferred into a 1.5 ml eppendorf tube and lithium phenyl-2,4,6-trimethylbenzoylphosphinate (LAP) photoinitiator is added at a final concentration of 0.1%. The bioink loaded into a 1 ml low dead volume syringe provided with 250 μm nozzle and casted into 4×2 mm silicon rubber mould between two 10×10×1 mm glass slide coverslips in a 100 mm plastic dish. The bioink was then photocrosslinked inside the mould at 20 mW/cm², 405 nm for 60 seconds using a UV box (BioLambda, Sao Paulo, Brasil). To prepare the control sample, cells were seeded on a tissue culture plate plastic at 1.6×10⁵ cell/cm², detached with trypsin/EDTA at day 7, mixed with GelMA6%+0.1% LAP and photocrosslinking induced with 405 nm wavelength at 20 mW/cm² for 60 seconds. The obtained bioscaffolds (shown in FIG. 5) were removed from the mould using sterile tweezers and placed into a 24 low attachment cell culture plate containing PBS.

The metabolic activity of the bioscaffolds were evaluated 24 hours after the photocrosslinking procedure according to the following protocol. CellTiter-BlueÆ Reagent (Promega, Madison, WI, USA) was used according to manufacturer's instructions. A total volume of 1.0 mL was used per sample. The cellular scaffolds were incubated for 3 hours at 37° C. 5% CO2 with the CellTiter solution diluted in cell culture media and the solution collected and measured in a CLARIOStar plate reader at 550+/−15 excitation nm and 600+/−20 nm emission using the same gain for all readings.

Bioscaffolds were maintained for 21 days in human adipose derived chondrogenic media [high glucose DMEM (Sigma-Aldrich), 100 U/ml Penicillin and 100 μg/ml Streptomycin solution (Gibco), 2 mM L-Glutamine (Gibco), 15 mM HEPES (Gibco), 10 ng/ml Transforming Growth Factor B3 (TGF (3) and 10 ng/ml Bone Morphogenic Factor 6 (BMP6) (R&D S Systems Inc., Minneapolis, MN, USA)]. The media was replenished every 72 h.

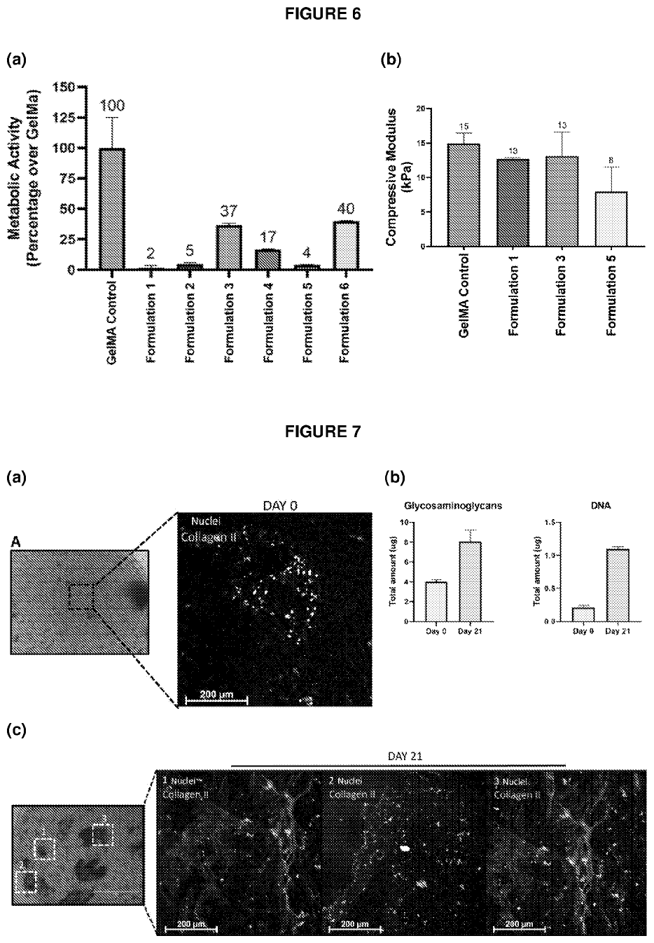

The results of the metabolic activity assay are shown in FIG. 6a. Each of the tested photocrosslinked formulations presented actively metabolic cells after the liquification and photocrosslinking steps with Formulation 6 showing the highest cell viability.

The compressive modulus (10-15% strain) of the Alg-MA-RGD scaffolds prepared from Formulations 1, 3 and 5 and control GelMA 6% scaffold was determined. The results are shown in FIG. 6b. Formulations 1 and 3 were found to have similar compressive modulus to GelMA 6%.

Chondrogenesis is evaluated via glycosaminoglycan content and immunohistological analysis of Collagen Type II according to the following protocols.

Glycosaminoglycan (GAG) quantification was performed by dissolving cellular and acellular scaffolds in papain buffer (sodium phosphate buffer 0.2 M, cysteine 0.01 M, NaH₂PO₄·1H₂O 0.2 M, EDTA C₁₀H₁₄N₂Na₂O₈·2H₂O 0.01 M, papain 250 μg/mL) at 65° C. for 5 h. Samples were then centrifuged at 10,000 g for 10 min at RT and the surnatant transferred in clean tube for dimethylmethylene blue (DMMB) reaction by mixing samples and DMMB solution in a 1:5 ratio. Purified chondroitin sulfate (Sigma Chemical) was used as a reference standard. The absorbance was immediately read using a ClarioStar plate reader at 525 nm and 595 nm absorbance, and the ratio 525/595 was determined. Based on the standard curve, the GAG amount was expressed in ug and multiply for the dilution factor coming from the papain digestion.

DNA quantification was performed from papain digested scaffolds using Quant-IT PicoGreen dsDNA Reagent Kit (Molecular probes) following Manufacturer's instructions.

For the immunohistological analysis, 3D scaffolds were fixed in 1% paraformaldehyde (Santa Cruz Biotechnology, Dallas, TX, USA) for 4 h at room temperature, embedded in O.C.T. TM Compound (Tissue-Tek, Sakura, Leiden, Netherlands) and after several washes in PBS 1× flash frozen in liquid nitrogen. Cryosections of 7 μm thickness were cut along the axial plane to consider the entire scaffold from top to bottom. The cryosections were mounted onto SuperFrostPlus adhesion glass slides (Thermo Scientific, Waltham, MN, USA) for staining and imaging. For fluorescence analysis, slices were washed three times in PBS 1× and permeabilized for 15 min in PBS 1×-0.1% TritonX-100 (PBT). Antigen retrieval was performed by adding 1 mg/ml hyaluronidase (Sigma-Aldrich, #H6254) diluted in PBS 1× and incubating 30 min at room temperature. After three washes 5 min each in PBS 1×, samples were dropped in blocking solution (10% goat serum diluted in PBT) for 60 min at room temperature and then incubated overnight at 4° C. with mouse anti-human collagen II (#∥6B3, DSHB). The day after, samples were washed three times for 10 min each and secondary antibodies both diluted 1:100 in blocking solution were added: anti-mouse Alexa Fluor 647 IgG H+L (#715-605-151, Jackson Immuno Research) and incubated for 2 h at room temperature. After three washes 5 min each in PBS 1×, nuclei were stained by incubation with 5 μg/mL DAPI (Thermo Fisher Scientific Inc.) diluted in PBS 1× for extra 60 min at room temperature. The sections were washed three times 5 min each in PBS 1×, mounted with Fluoromount-G (Southern Biotech, Birmingham, AL, USA). Images were taken with NikonA1R confocal microscope (Nikon, Amsterdam, Netherlands) using a Nikon Plan VC 20×DIC N2 N.A. 0.75 objective lens and “Scan large image” from NIS-Elements software tool was used to image a larger field of view. Digital images were processed using NIS-Elements software (Nikon, Amsterdam, Netherlands) and Photoshop2020 software (Adobe) was used to assemble the figure panels.

The chondrogenic capability of Alg-MA-RGD scaffolds was verified and data obtained from Formulation 1 are shown in FIG. 7. The scaffolds showed no presence of collagen II at the start of the experiment as shown in FIG. 7Bi. After 21 days of chondrogenic stimuli according to the above procedure, the scaffolds exerted an increase in glycosaminoglycans and DNA total content, illustrated in FIG. 7Bii. The immunohistological analysis in FIG. 7Biii demonstrates the accumulation of collagen type II, the main marker of hyaline cartilage, in the scaffolds.

### Comparative Example 1. Storage Modulus Properties of Blends of Alginate, Alginate Methacrylate and Alginate-RGD

The storage modulus properties of blends (mixtures) of alginate, alginate methacrylate (Alg-MA) and alginate-RGD (Alg-RGD) were assessed. Alg-RGD was sourced from Novatach™ (NovaMatrix®). Solution mixtures were preparing containing varying weight ratios of alginate, Alg-MA and Alg-RGD. Alg-RGD content was kept at 20% for all mixtures while alginate and Alg-MA were varied from 10% to 70% respectively.

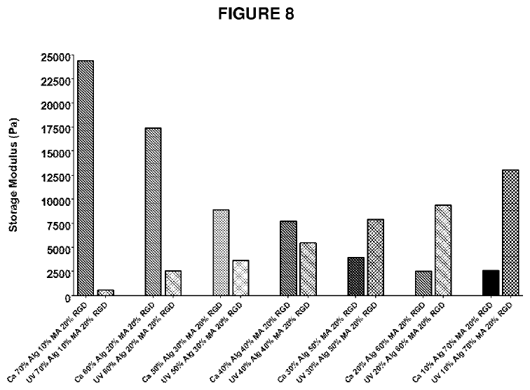

The blends were subjected to calcium crosslinking and photocrosslinking and the storage modulus for each determined following the respective procedures in Example 2. The results are shown in FIG. 8. In general, the calcium crosslinking ability and photocrosslinking ability were inversely related for each blend. None of the formulations tested achieved a target storage modulus of 10 kPa for both crosslinking modes.

### Comparative Example 2

The present inventors have evaluated the characteristics of the Universal Polymer by using an ionic crosslinker (CaCO₃) to provide information on the material's stiffness, compressive modulus, degradation, and crosslinking rate; 2) to provide the optimal concentration of CaCO₃ to be used in the biological investigation.

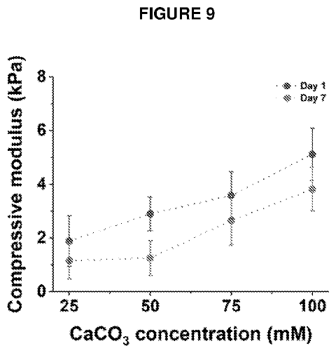

The present inventors evaluated and optimised the concentration of CaCO₃ and EDTA decrosslinker using batch #446. Unipolymer samples were prepared at different concentrations of the chemical crosslinker CaCO₃-GDL (Glucono-δ-lactone), maintaining the ratio 1:2 of CaCO₃:GDL for all the concentrations. GDL is used to dissociate the Ca2+ ions to allow the ionic crosslinking of the alginate. The concentrations of the samples were 25, 50, 75, and 100 mM of CaCO₃. Samples were chemically crosslinked in a cylindrical shape (4 mm diameter×2 mm high) using a PDMS mould. Samples were incubated for 7 days in cell proliferation media. FIG. 9 shows the compressive modulus of the Universal Polymer calculated between 10-15% strain at Day 1 and 7.

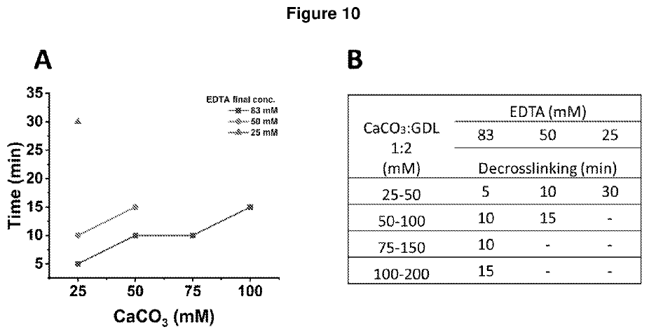

Next, the same samples were exposed to the phase change (decrosslinking) process by adding the chelator agent EDTA. FIG. 10 shows the decrosslinking of the Universal Polymer with EDTA at different concentrations.

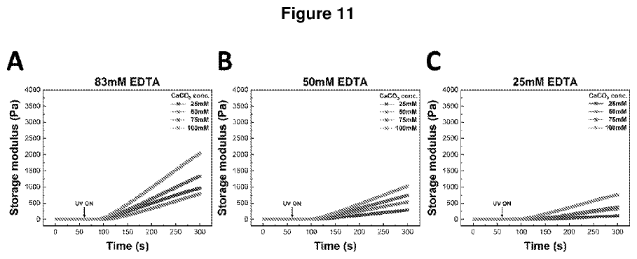

Once the samples were decrosslinked (phase changed), they were photocrosslinked in the rheometer using 405 nm UV light at 20 mW/cm² (inc. LAP 0.1% w/v) to investigate their storage modulus via photorheology. FIG. 11 shows the storage modulus of the samples decrosslinked with the EDTA decrosslinking solution at three different concentrations.

### Comparative Example 3

The present inventors have evaluated the biological characteristics of the Universal Polymer to provide information on the biocompatibility of the Universal Polymer ionically crosslinked with CaCO₃ before phase change, after phase change with three different decrosslinkers and after photocrosslinking.

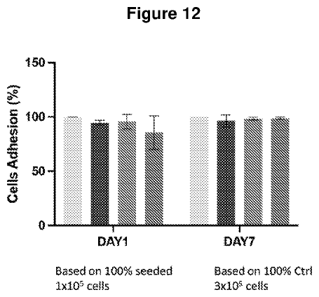

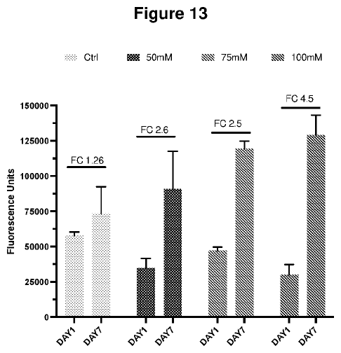

Stage A-Culture Substrate: Cell viability of the Universal Polymer was investigated at different concentrations of ionic crosslinking. At first, samples were chemically crosslinked as layers of 100 μl onto a well of ultra-low attachment 24-well plate at the concentrations of 50-75-100 mM of CaCO₃ and compared to control cell culture plastic. Once crosslinked, cell media was added to the layers and were incubated for 72 h hours. A total of 1×10⁵ cells (hADSC) were added onto the layers and incubated for further 1 and 7 days. Cell attachment (FIG. 12) and viability (FIG. 13) were assessed at both Day 1 and 7.

Stage B-Phase Change: Further analysis were performed at Day 7, where the Universal Polymer layers containing the cells (hADSC) undergo the phase change process for 15 minutes at 37 C, via the addition of three different decrosslinkers:
1) EDTA 83 mM dissolved in MilliQ pH 8.9
2) EDTA 83 mM dissolved in CellCulture Media pH 8.0
3) Mixture-Sodium citrate buffer composition: 150 mM NaCl, 330 mM sodium citrate, 120 mM EDTA PH 8.8.

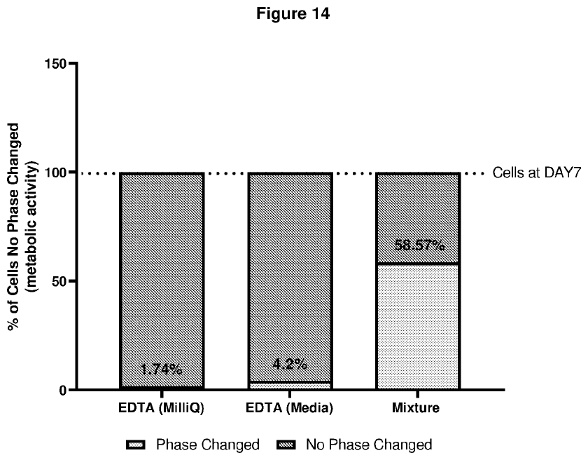

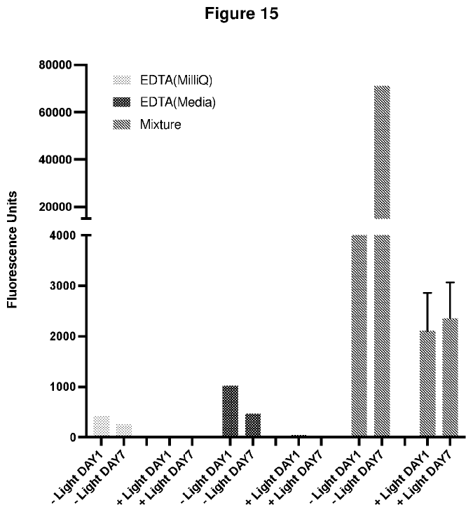

The cells viability was then assessed by replating the liquified material (containing decrossliked Univeral Polymer and hADSC) into tissue culture plates via metabolic activity measurement (FIG. 14). The mixture decrosslinker gives the highest cells viability over the EDTA conditions.

Stage C-Delivery of Bioscaffold: after phase change, 25 μl samples transferred into a 96-well culture plate and photocrosslinked (+Light) using 405 nm UV light at 20 mW/cm² (inc. LAP 0.1% w/v). Cell viability via metabolic activity measurement 24 hrs after (DAY1) and after 7 days (DAY7) was then evaluated (FIG. 15). Uncrosslinked samples were used as well for comparison (−Light). The mixture decrosslinker gives the highest cell viability over the EDTA conditions.

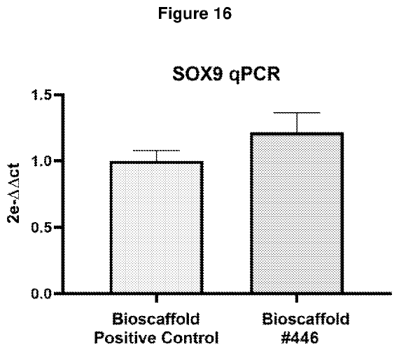

Bioscaffolds were induced towards chondrogenic differentiation for 7 Days. Gene expression analysis showed expression of SOX9, the master regulator of chondrogenesis, after 7 Days of induction in batch #446 similar to a control bioscaffold where the same cell population was embedded in a standard hydrogel material (GelMA), photocrosslinked with the same parameters (405 nm wavelength at 20 mW/cm² for 60 sec) (FIG. 16).

1. A functionalised polymer comprising a polymer, wherein the polymer is alginate, and wherein the alginate polymer is partially functionalised with:
a plurality of photocrosslinkable moieties linked to the alginate polymer, each photocrosslinkable moiety comprising a reactive functionality capable of photocrosslinking, wherein about 16.0% to about 68.8% of the hydroxyl groups of the alginate polymer are functionalised with the photocrosslinkable moieties; and
a plurality of cell adhesion moieties each linked to the alginate polymer via a linker, each cell adhesion moiety comprising a cell adhesion motif; wherein, the linker is derived from a photocrosslinkable moiety linked to the alginate that has reacted with a peptide for cell adhesion, and wherein from about 2% to about 9% of the photocrosslinkable moieties are functionalised with the plurality of cell adhesion moieties; and
wherein the functionalised polymer further comprises a plurality of functional groups capable of ionic crosslinking with an ionic crosslinking agent
optionally wherein:
i) the polymer is partially functionalised to an extent such that the functionalised polymer is capable of a liquid to solid phase change caused by an ionic crosslinking agent;
ii) the polymer is partially functionalised to an extent such that the functionalised polymer is capable of a solid to liquid phase change caused by a chelator chelating an ionic crosslinking agent;
iii) the polymer is partially functionalised with the plurality of cell adhesion moieties to an extent such that the functionalised polymer is capable of adhering cells; and/or
iv) the polymer is partially functionalised to an extent such that the functionalised polymer is capable of a liquid to solid phase change caused by photocrosslinking.

2.-5. (canceled)

6. The functionalised polymer of claim 1 comprising partially functionalised alginate polymer with:
the plurality of photocrosslinkable moieties linked to the polymer in uncrosslinked form; and
the plurality of cell adhesion moieties optionally in association with cells bound thereto; and
wherein the alginate polymer is crosslinked with a divalent cationic crosslinking agent, optionally wherein the cationic crosslinking agent is divalent calcium, optionally wherein the cationic crosslinking agent is divalent calcium.

7. (canceled)

8. The functionalised polymer of claim 1, wherein the photocrosslinking moieties comprise one or more of acryloyl, methacryloyl, acrylate, methacrylate, acrylamide, methacrylamide, allyl ether, maleimide, vinyl sulfone, NHS ester and vinyl ether.

9. The functionalised polymer of claim 1, wherein the cell adhesion moieties each comprise an integrin binding motif, optionally wherein the cell adhesion moieties are selected from the group consisting of RGD, RGDS, GGGGRGDSP, GRGDSP, or GRGDS, further optionally wherein the integrin binding motif is RGD.

10. The functionalised polymer of claim 1, further comprising cells adhered to the cell adhesion moieties, optionally wherein the cells are cells that have chondrogenic, osteogenic and/or adipogenic potential, further optionally wherein the cells are adult stem cells, preferably selected from mesenchymal stem cells and adipose derived stem cells (ADSCs).

11.-12. (canceled)

13. The functionalised polymer of claim 1, wherein the alginate polymer has an M/G ratio in the range of about 0.30 to about 2.60, optionally, in the range of about 0.6 to about 1.30, further optionally in the range of about 0.6 to about 1.4, further optionally wherein the alginate polymer has an M/G ratio of about 0.64 to 1.30.

14. The functionalised polymer of claim 1, wherein the alginate polymer has an average molecular weight of from about 68 kDa to about 780 kDa, optionally from about 200 kDa to about 400 kDa.

15.-18. (canceled)

19. The functionalised polymer of claim 1, wherein the reactive functionality capable of photocrosslinking is methacrylate, optionally wherein the plurality of photocrosslinkable moieties are a plurality of methacrylate groups.

20. (canceled)

21. The functionalised polymer of claim 1, wherein the plurality of photocrosslinkable moieties are each derived from a reagent for providing the photocrosslinkable moiety that has reacted with a hydroxyl group of the polymer.

22.-23. (canceled)

24. The functionalised polymer of claim 1, wherein the plurality of cell adhesion moieties are each conjugated to a linker via a thioether bond, optionally wherein the photocrosslinkable moiety and the peptide for cell adhesion have undergone a thiol-Michael addition reaction.

25.-26. (canceled)

27. A method for preparing the functionalised alginate polymer of claim 1, the method comprising:
providing an alginate polymer partially functionalised with a plurality of photocrosslinkable moieties, each photocrosslinkable moiety comprising a reactive functionality capable of photocrosslinking, wherein about 16.0% to about 68.8% of the hydroxyl groups of the alginate polymer are functionalised with the photocrosslinkable moieties; and
reacting a peptide for cell adhesion with from about 2% to about 9% of the photocrosslinkable moieties present on the polymer, to thereby provide the plurality of cell adhesion moieties linked to the alginate polymer via a linker;
thereby providing the functionalised polymer.

28. A method for preparing the functionalised alginate polymer of claim 1, the method comprising:
providing an alginate polymer comprising a plurality of functional groups, wherein the plurality of functional groups are a plurality of hydroxyl groups;
reacting a reagent for providing a photocrosslinkable moiety with plurality of functional groups present on the polymer from about 16.0% to about 68.8% of the hydroxyl groups of the alginate polymer, to thereby provide a polymer partially functionalised with a plurality of photocrosslinkable moieties; and
reacting a peptide for cell adhesion with from about 2% to about 9% of the photocrosslinkable moieties present on the polymer, to thereby provide the plurality of cell adhesion moieties linked to the alginate polymer via a linker;
thereby providing the functionalised polymer.

29.-34. (canceled)

35. The method of claim 27, wherein the step of reacting a peptide for cell adhesion comprises;
reacting the peptide for cell adhesion with a portion of the plurality of photocrosslinkable moieties present on the polymer via a thiol-Michael addition reaction; and/or
reacting a peptide for cell adhesion comprises reacting the peptide for cell adhesion with from about 4% to about 14% of the photocrosslinkable moieties present on the polymer.

36. (canceled)

37. A polymer composition comprising the functionalised polymer of claim 1, and an aqueous solution, optionally wherein
i) the aqueous solution is a buffer solution; and/or
ii) the polymer composition comprises the functionalised polymer in an amount of from about 3% w/v to about 10% w/v, based on the volume of aqueous solution.

38.-39. (canceled)

40. A method for forming a liquified alginate polymer composition comprising cells, the method comprising:
providing a tissue sample comprising cells or cells isolated therefrom;
contacting the sample or cells with the functionalised polymer of claim 1 in binding conditions, said binding conditions being conditions that enable binding of cells in the sample to the functionalised polymer, so that said cells are bound to the functionalised polymer;
culturing the cells bound to the polymer under conditions and for a time that allows, or causes, the cell number to increase;
providing conditions to induce a solid to liquid phase change of the functionalised polymer;
optionally wherein, a photoinitiator, such as LAP, is added to the liquefied alginate polymer composition.
thereby forming a liquified alginate polymer composition comprising cells from a tissue sample.

41.-72. (canceled)

73. A method of cell culture comprising:
providing an alginate polymer, wherein the alginate polymer is ionically crosslinked and partially functionalised with:
a plurality of photocrosslinkable moieties linked to the alginate polymer in uncrosslinked form, each photocrosslinkable moiety comprising a reactive functionality capable of photocrosslinking, wherein about 16.0% to about 68.8% of the hydroxyl groups of the alginate polymer are functionalised with the photocrosslinkable moieties; and
a plurality of cells bound to the alginate polymer by adhesion moieties each linked to the polymer via a linker, each cell adhesion moiety comprising a cell adhesion motif, wherein, the linker is derived from a photocrosslinkable moiety linked to the alginate that has reacted with a peptide for cell adhesion, and wherein from about 2% to about 9% of the photocrosslinkable moieties are functionalised with the plurality of cell adhesion moieties;
culturing the cells bound to the crosslinked alginate polymer under conditions and for a time that allows, or causes, the cell number to increase;
optionally, wherein the alginate polymer is cationically crosslinked, liquifying the ionic crosslinked alginate polymer by addition of a chelating agent, such as EDTA, for the cationic crosslinking agent to form a liquified alginate polymer composition comprising cells;
optionally, applying the liquified alginate polymer composition comprising cells to a substrate; and
further optionally, photopolymerising the liquified alginate polymer composition comprising cells to cause the photocrosslinkable moieties to photocrosslink the alginate polymer and form a solid cross-linked structure comprising the cells.

74. The method of claim 73, wherein the crosslinked alginate polymer is formed by a method comprising:
(a) providing an alginate polymer partially functionalised with:
a plurality of photocrosslinkable moieties linked to the polymer, each photocrosslinkable moiety comprising a reactive functionality capable of photocrosslinking, wherein about 16.0% to about 68.8% of the hydroxyl groups of the alginate polymer are functionalised with the photocrosslinkable moieties; and
a plurality of cell adhesion moieties each linked to the polymer via a linker, each cell adhesion moiety comprising a cell adhesion motif, wherein, the linker is derived from a photocrosslinkable moiety linked to the alginate that has reacted with a peptide for cell adhesion, and wherein from about 2% to about 9% of the photocrosslinkable moieties are functionalised with the plurality of cell adhesion moieties;
(b) adding divalent cationic crosslinking agent, such as calcium ions, to crosslink the alginate polymer to induce a phase change of the alginate polymer from liquid to solid; and
(c) binding cells to the plurality of cell adhesion moieties linked to the crosslinked alginate polymer having a solid crosslinked structure.

75.-85. (canceled)

86. A method for treating an individual comprising:
administering a composition prepared by the method of claim 40, or a subsequent 3D bio printed tissue, to the individual,
thereby treating the individual, optionally wherein
i) the composition is formed from a tissue sample obtained from the individual; and/or
ii) the composition is formed from a tissue sample obtained from an infrapatellar fat pad of the individual; and/or
iii) the composition is administered by injection, extrusion or 3D printing; and/or
iv) the individual has a condition of an articular surface requiring repair or restoration; and/or
v) the composition is administered arthroscopically, preferably with ultrasound or imaging guidance; and/or
vi) the delivered composition is hardened by the activation of a photoinitiator, optionally wherein the photoinitiator is activated with visible light, further optionally wherein the photoinitiator is LAP, further optionally wherein a 405 nm light source at 20 mW/cm² is applied for 1 minute.

87.-105. (canceled)

106. The functionalised polymer of claim 1, wherein the functionalised polymer is capable of forming a solid upon photocrosslinking having a storage modulus of at from about 5 kPa to about 50 kPa, optionally from about 7 to about 37 kPa.

107. The method of claim 28, wherein the step of reacting a peptide for cell adhesion comprises:
reacting the peptide for cell adhesion with a portion of the plurality of photocrosslinkable moieties present on the polymer via a thiol-Michael addition reaction; and/or
reacting a peptide for cell adhesion comprises reacting the peptide for cell adhesion with from about 4% to about 14% of the photocrosslinkable moieties present on the polymer.
